(19) United States
(12) Patent Application Publication
(10) Pub. No.: US 2024/0415654 A1
(43) Pub. Date: Dec. 19, 2024

(54) TISSUE ANCHORS AND TECHNIQUES FOR USE THEREWITH

(71) Applicant: Edwards Lifesciences Innovation (Israel) Ltd., Caesarea (IL)

(72) Inventors: Yaron Herman, Givat Ada (IL); Noam Glezer, Ashkelon (IL); Gidon Pesach, Kfar Vitkin (IL); Eran Hoffer, Yahud (IL); Aviv Galon, Tel Aviv (IL); Carmel Peleg, Pardes Hana-Karkur (IL); Eyal Shaolian, Newport Beach, CA (US); Ehud Aviv (formerly Iflah), Costa Mesa, CA (US); Travis Zenyo Oba, Yorba Linda, CA (US); Ofry Efraim Yossef, Tel-Aviv (IL); Sam Shafigh, Irvine, CA (US); Bar Eytan-Vaisman, Gedera (IL); Matthew Aaron Sussman, Laguna Hills, CA (US); Eva Adriana de la Cruz Romito, Irvine, CA (US); Omar Fawzi Azanki, Coto de Caza, CA (US); Julia Akiko Roche, Costa Mesa, CA (US); Andrew Charles May, Irvine, CA (US); Alana Tyler Stein, Irvine, CA (US)

(21) Appl. No.: 18/750,978

(22) Filed: Jun. 21, 2024

Related U.S. Application Data
(63) Continuation of application No. PCT/IB2022/062187, filed on Dec. 14, 2022.
(60) Provisional application No. 63/346,823, filed on May 28, 2022.
(60) Provisional application No. 63/292,317, filed on Dec. 21, 2021.

(51) Int. Cl. A61F 2/24; A61B 17/04

(52) U.S. Cl. CPC A61F 2/2466; A61B 2017/0464

(57) ABSTRACT

A system comprises an anchor (20) comprising an inner helical tissue-engaging element (24); an outer helical tissue-engaging element (22); and a head assembly (29). The head assembly comprises a first head (25), fixed to a proximal end of the inner tissue-engaging element, and a second head (26), fixed to a proximal end of the outer tissue-engaging element. The system further comprises a driver (28), reversibly interfacing with and engaging the first head and the second head, in order to anchor the anchor in the target tissue. Other applications are also described.

### CROSS-REFERENCES TO RELATED APPLICATIONS

This application is a continuation of International Patent Application No. PCT/IB2022/062187, filed Dec. 14, 2022, with claims the benefit of U.S. Patent Application 63/292,317, filed on Dec. 21, 2021, and of U.S. Patent Application 63/346,823, filed on May 28, 2022, the entire disclosures all of which are incorporated by reference for all purposes.

### BACKGROUND

Tissue anchors are used to anchor elements, such as implantable devices, to tissues such as bone and soft tissues. Some tissue anchors are used to attach two tissues to each other. Tissue anchors may be made of various materials. They may be designed to be permanent, or they may be temporary and resorbable. They may have various shapes and designs. For example, some tissue anchors may define a shaft and screw thread therearound, while other tissue anchors may be shaped so as define a helical tissue-coupling element without a shaft. A variety of tissue anchors can be configured for various tissue types, and these may differ for anchors for soft vs. hard tissue.

For example, some heart valve treatment devices may require several anchors to attach the device to the tissue surrounding the valve being treated. The effectiveness of the device may be directly tied to the strength of these anchors, especially for annular devices that need to be tightened around the valve annulus and may be under hemodynamic stress. In addition, the anchors must also be easy to place due to the challenging nature of the operation, the importance of the anchor placement location, and the large number of anchors that may be required.

### SUMMARY

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure can be included in the examples summarized here.

Herewithin are disclosed various applications of tissue anchors having more than one tissue-engaging element.

For some applications, the tissue anchors have a head assembly and a helical tissue-engaging element (e.g., a first tissue-engaging element), and an additional or supplementary tissue-engaging element.

For some applications, the helical tissue-engaging element can include, e.g., a radioactive substance or a polymeric substance, and can have a tissue-piercing tip at the distal end.

For some applications, a driver can be provided for transluminally advancing and anchoring the anchor.

For some applications, the additional tissue-engaging element can also be a helical tissue-engaging element. For such applications, the first helical tissue-engaging element can be disposed within the additional helical tissue-engaging element (e.g., coaxially).

For some applications, the various tissue-engaging elements herein can comprise one or more hooks, barbs, darts, staples, clips, protrusions, arms, expandable portions, threaded portions, rivets, pledgets, helixes, screws, screw-like portions, combinations of two or more of these, etc.

For some applications, the additional helical tissue-engaging element can have the same pitch and handedness as the first helical tissue-engaging element. For some applications, the additional helical tissue-engaging element can have a different pitch and/or opposite handedness as the first helical tissue-engaging element.

For some applications, both helical tissue-engaging elements are fixed to the same head. For some applications, the first helical tissue-engaging element is fixed to one head, and the additional helical tissue-engaging element is fixed to another head. For some applications, one, e.g., a first, head can include an outer cylinder, and the other, e.g., the second, head can include an independently moveable inner core that interfaces with the first head.

For some applications, the second head can fit within the first head when both helical tissue-engaging elements are completely inserted into the tissue. For some applications, the two heads can cooperate in an interlocking fashion to provide additional stability and force distribution within the tissue.

For some applications, the helical tissue-engaging element serves as a primary tissue-engaging element, and the additional tissue-engaging element serves as a supplementary tissue-engaging element. For some applications, the supplementary tissue-engaging element can be a different type of tissue-engaging element, e.g., a disc with barbs, a set of hooks, or a set of tissue-engaging prongs.

For some applications, the primary tissue-engaging element is attached to a first head of the head assembly. For some applications, the primary tissue-engaging element extends away from the first head, helically around an elongate space that is disposed along a longitudinal axis of the anchor and can terminate at a sharpened distal tip of the helical tissue-engaging element.

For some applications, the supplementary tissue-engaging element includes a plurality of prongs, which are attached to a second head of the head assembly. For some applications, the second head can be coupled to the first head in a manner in which the second head is rotatable with respect to the first head but is axially fixed with respect to first head.

For some applications, the prongs extend from the second head distally through the elongate space and are arranged with respect to the helical tissue-engaging element such that rotation of the first head with respect to the second head feeds the prongs laterally outward between progressively proximal turns of the tissue-engaging element.

For some applications, the supplementary tissue-engaging element is neither helical nor disposed within the primary tissue-engaging element. For example, the supplementary tissue-engaging element can be a set of articulatable arms with hooks for stabilizing the head of the anchor. For some applications, the hooks and arms can have a disengaged position oriented away from the primary helical tissue-engaging element as the primary helical tissue-engaging element is advanced into the tissue by the driver.

For some applications, the hooks and arms can be held in the disengaged position, e.g., by the driver used to transluminally advance the tissue anchor toward the tissue. After the primary tissue-engaging element has been fully anchored into the tissue, the set of hooks and arms can be released to their engaged position substantially lateral to the head. The supplementary hooks and arms thus provide additional force distribution and stabilization for the tissue anchor.

For some applications, a supplementary tissue-engaging element can be configured as a ring disposed axially between the anchor head and the primary tissue-engaging element, e.g., a helical tissue-penetrating screw. For some applications, the ring can have barbs, tines, or hooks on the outer circumference, which can protrude radially outward beyond the head.

For some applications, a gap can be present between the ring and the primary tissue-engaging element.

For some applications, the supplementary tissue-engaging element can provide additional lateral stability to the tissue anchor. For some applications, the ring can rotate freely and independently as the primary tissue-engaging element is anchored into the tissue. For some applications, the ring can then become fixed in place when the outer edges come into contact with the tissue surface.

For some applications, a ring-shaped supplementary tissue-engaging element can be fixedly attached to the anchor between the head and the primary tissue-engaging element. For some applications, the ring can have a number, e.g., 3, 4, 6, or 8, of protrusions, each of which forms a lobe. For some applications, each lobe can have a circumferential directionality that facilitates anchoring of the anchor in the target tissue, and/or that inhibits extrusion of the anchor.

For some applications, a distal and/or a proximal circumferential surface of each lobe is beveled. The beveling may facilitate entry into the tissue. The circumferential directionality may predispose the ring to resist rotation within the tissue.

For some applications, a system and/or a method for verifying a placement of the tissue anchor in a tissue of a subject is provided. The system and/or method can apply to any of the tissue anchors described herewithin.

For some applications, the head (or head assembly) of the relevant anchor can define an internal hollow, and a port opening into the hollow, the internal hollow being open at a tissue-facing surface of the head. For some applications, a driver is used to anchor the anchor by driving the tissue-engaging element(s) of the anchor into the tissue in a manner that places the tissue-facing surface of the head in contact with the tissue. For some applications, the driver can include a shaft that defines a channel in fluid communication with the port.

For some applications, a contrast agent is then dispensed via the channel and the port into the hollow. A radiographic imaging procedure can then be performed to check for the presence of the radiopaque contrast agent in the hollow and/or outside of the hollow. If the anchor has been optimally and/or completely anchored, the contrast agent can be observed to have been retained in the hollow (e.g., due to contact between the tissue-facing surface and the tissue). In contrast, if the anchor has been anchored suboptimally and/or incompletely, the tissue-facing surface of the head may not contact the surface of the target tissue, such that contrast agent dispensed into the hollow rapidly leaks out of the hollow.

For some applications, one or more tissue anchors herein can be configured to comprise one or more mechanisms (e.g., additional tissue-engaging elements or features, adhesives, sutures, friction enhancing elements, barbs, clasps, grippers, etc.) to enhance the tissue-holding strength and/or to facilitate the utility of the anchor.

For some applications, the tissue anchor includes a head, a helical tissue-engaging element, and a torque limiter, in which the torque limiter is configured to limit the amount of torque that can be applied by a driver to the helical tissue-engaging element. The torque limiter can include a variety of mechanisms, e.g., a slip clutch, a gear set, or a shear pin.

For some applications, in which the torque limiter uses a slip clutch mechanism, the slip clutch can include a pair of intertwined torsion springs lying on a plane within a base of the anchor head, each spring with a respective pawl at its outer extremity. For some applications, the pawls are configured to engage a series of notches on an inner surface of the base, which is fixedly coupled to the tissue-engaging element.

For some applications, when the driver screws the tissue-engaging element into a tissue, the pawls slip past the notches as the torque is transferred from the driver via the head, i.e., the base, to the tissue-engaging element. Once the predetermined amount of torque has been transferred to the tissue-engaging element, the pawls will begin to slip on the notches, such that further torque will be released rather than transferred to the tissue-engaging element.

For some applications, the tissue anchor can include a head, a tissue-engaging element, e.g., a helical tissue-engaging element, and a tubular sleeve, extending distally from the head and partially or completely enclosing the tissue-engaging element. The sleeve can include, e.g., a woven or knitted fabric, a braid, a mesh or a flexible film.

For some applications, e.g., in which the sleeve includes a braid or mesh, the material can be nitinol or another metal. For some applications, e.g., in which the sleeve includes a flexible film, the material can be silicone or another biocompatible polymer.

For some applications, the sleeve can be reinforced with a helical wire or with short prongs, i.e., shorter than the tissue-engaging element.

For some applications, e.g., in which the sleeve is reinforced with the helical wire, the wire is configured to provide additional support and/or elastic axial compressibility to the sleeve.

For some applications, a driver is used to screw the tissue-engaging element into the tissue, such that the sleeve remains outside of the tissue, and becomes compressed between the anchor head and a surface of the tissue as the tissue-engaging element advances into the tissue. For some applications, the sleeve provides extra lateral support to the head and may help prevent anchor pull-out.

For some applications, e.g., in which the anchor further includes short prongs, the prongs can be disposed either between the sleeve and the tissue-engaging element, or can be woven into the fabric, mesh, or film of the sleeve. For some applications, e.g., in which the prongs are disposed between the sleeve and the tissue-engaging element, prongs can provide additional later support to the anchor by penetrating superficial layers of the tissue.

For some applications, the tissue anchor includes a head, a first helical tissue-engaging element, and a second helical tissue-engaging element. For some applications, the first and second tissue-engaging elements are coaxial and extend distally from the head. For some applications, the first tissue-engaging element can be disposed medially to the second tissue-engaging element. For some applications, the tissue-engaging elements can include stainless steel or another non-expandable metal. For some applications, the second tissue-engaging element can include an expandable or elastic shape memory material such as nitinol.

For some applications, prior to deployment, the tissue anchor is disposed within a delivery capsule that prevents expansion of and compresses the second tissue-engaging element to have a narrower diameter than its natural, i.e., shape memory diameter.

For some applications, the delivery capsule is advanced to the tissue by a driver, to which it is coupled. As the driver advances, i.e., rotates, the tissue anchor into the tissue, the first tissue-engaging element advances axially into the tissue, whereas the second tissue-engaging element, upon release from the capsule, advances axially while concurrently expanding laterally. Thus, upon full deployment, the second tissue-engaging element is disposed laterally to the first tissue-engaging element and has an outer diameter greater than the head, providing additional strength to the anchor.

For some applications, the tissue anchor includes a head and a helical tissue-engaging element, including a shape memory wire, either or both of which can be expandable.

For some applications, the head, in its native state, can form a single, flat coil, or can have multiple coils that provide a tissue-contact surface analogous to that of other tissue anchors.

Prior to deployment, the tissue anchor can be disposed within a delivery tool. For some applications, the delivery tool includes a tube having an internal diameter that is smaller than the deployed width of the anchor head and/or helical tissue-engaging element, such that the anchor is constrained within the delivery tool in an elongated shape.

For some applications, a driver within the delivery tool is configured to engage the proximal end of the anchor, i.e., the proximal end of the head, and to thus push the anchor out of the delivery tool into the tissue.

For some applications, as the anchor exits the delivery tool, the anchor assumes its native state, and stress stored in the elongated form of the anchor can be released, thus enabling the tissue-engaging element to advance into the tissue.

For some applications, the proximal end of the head includes a crossbar. The crossbar can be configured to be grasped by a tool for anchor tightening and/or retrieval. For some applications, the crossbar can be used to couple another component to the anchored anchor.

For some applications, the tissue anchor includes a head, a primary tissue-engaging element, e.g., a helical screw, and a secondary tissue-engaging element including a set of prongs. For some applications, the prongs of the secondary tissue-engaging element can be disposed circumferentially around the primary tissue-engaging element. For some applications, the prongs can include a shape memory material such as nitinol, such that in the resting state, each prong is shaped as a proximally-curving hook or barb.

For some applications, the secondary tissue-engaging element can further include a collar disposed between the head and the primary tissue-engaging element to which the prongs are attached. For some applications, the collar can include a lip or tongue. Prior to delivery, the anchor is disposed within a delivery capsule having axial grooves in which the prongs are disposed, and a helical thread with a handedness and pitch that match the helical turns of the primary tissue-engaging element.

For some applications, the delivery capsule includes a longitudinal slit extending the length of the capsule. For some applications, the tongue of the collar can fit into the slit, configured to maintain the secondary tissue-engaging element in a rotationally stationary position during delivery.

For some applications, the delivery capsule is configured to couple to a driver, such that as the driver advances the primary tissue-engaging element along the helical threads into the tissue, the prongs advance axially outward along the axial grooves of the delivery capsule.

For some applications, the helical thread prevents the prongs from prematurely pushing the entire anchor forward out of the delivery capsule. For some applications, once the primary helical tissue-engaging element has been secured in the tissue, the delivery capsule is withdrawn, and the prongs assume their native hook-shaped configuration within the tissue.

For some applications, the tissue anchor includes a cylindrical head and set of prongs, e.g., 4, 6, or 8, coupled to the head. For some applications, the head defines an aperture extending therethrough along a longitudinal axis of the anchor. For some applications, the prongs can be arranged circumferentially around the head along the longitudinal axis. For some applications, the prongs can include a shape memory material, such that their native state is to extend laterally outward.

For some applications, each prong can include a pocket on its distal end. For some applications, the system can further include an insert, configured to extend through the aperture in the head. For some applications, the insert includes a head and a shaft having a sharp distal tip for piercing the tissue. For some applications, the insert further includes a set of fingers extending along the longitudinal axis that match the pockets of the prongs, such that each pocket fits in a corresponding finger. For some applications, the fingers hold the pockets medially around the insert prior to delivery. For some applications, during delivery, the anchor and insert are pushed into the tissue axially, the sharp tip of the insert used to pierce the tissue.

For some applications, the tissue anchor includes a head having an aperture along the longitudinal axis, and a tissue-engaging element including a hollow shaft, the shaft having windows along the lateral walls thereof.

For some applications, the anchor further includes a balloon disposed inside the hollow shaft, and a closure between the aperture of the head and the balloon.

For some applications, a delivery tool including a balloon expander is used to push the anchor, i.e., the tissue-engaging element, into the tissue. For some applications, at a point that the anchor is correctly positioned in the tissue, a fluid can be injected via the closure into the balloon, such that the balloon expands laterally through the windows, thus providing additional anchoring in the tissue.

For some applications, the fluid can include an inert liquid such as saline, or a hardenable resin such as epoxy. For some applications, the hardenable material can be cured by exposure to ultraviolet (UV) light, in which case the delivery tool can further include an optical fiber capable of transmitting UV light.

For some applications, the tissue anchor can include a suction-cup like device including a head having an axial port or ports, and a rigid cup having a flexible flange, the flange configured to align to the contours of the tissue.

For some applications, the anchor further includes a valve mechanism including a stopper. For some applications, the valve mechanism is disposed within the head, or between the head and the concave surface of the cup. Upon application of pressure to the valve, reduced pressure between the cup and a surface of the tissue causes fluid to exit via the port, thus creating a suction seal between the flange and the surface of the tissue.

For some applications, the tissue anchor can include a head, a tissue-engaging element, e.g., a helical tissue-engaging element, and an indicator ring. For some applications, the ring can be disposed at or near the distal end of the tissue-engaging element and is configured to slide proximally at the surface of the tissue as the tissue-engaging element is pushed, rotated, or screwed into the tissue. For some applications, when the tissue-engaging element is fully anchored in the tissue, the ring is disposed between the surface of the tissue and the head of the anchor, providing an indication of complete anchoring.

For some applications, the tissue anchor can include a head and a tissue-engaging element, e.g., a helical tissue-engaging element. For some applications, the anchor can include part of a system further including a delivery tool including a driver and a delivery capsule.

For some applications, the system can include an indicator wire having an acute bend toward a proximal part of the wire, and a radiopaque distal tip. For some applications, the indicator wire can include a shape memory material such as nitinol, and be configured to attach either to the head of the anchor or to a component of the delivery tool, e.g., the driver or the delivery capsule. For some applications, the indicator wire can be initially disposed within either the tissue-engaging element or within the delivery capsule.

For some applications, as the driver advances the tissue-engaging element into the tissue, the distal tip of the indicator wire moves laterally outward. For some applications, at a point in the anchoring process, the acute bend exits the delivery tool and turns proximally, assuming its resting position and indicating successful anchoring of the tissue-engaging element in the tissue.

For some applications, the tissue anchor can include a head and a tissue-engaging element, e.g., a helical tissue-engaging element. For some applications, the system can further comprise one or more prongs, e.g., having shape memory, and be configured to be inserted into the tissue prior to driving the tissue anchor into the tissue. For some applications, the prongs assume their memory shape within the tissue, and can serve to stabilize the driver at the surface of the tissue as the driver drives the anchor into the tissue. For some applications, the prongs can remain in the tissue as part of the tissue anchor. For some applications, the prongs are removed with the driver upon completion of anchoring the anchor in the tissue.

For some applications, the driver can comprise a self-driving mechanism for anchoring a tissue anchor within the tissue. For some applications, the driver can comprise a drive-head engageable with a tissue anchor.

For some applications, a winder at the proximal end of the driver can be used to apply torque to a torsion spring (e.g., a spiral torsion spring or a torsion bar) to wind up the spring and store potential energy in the spring. For some applications, a driveshaft serves as the torsion spring.

For some applications, a detent, disposed at either the proximal or distal end of the driver, is configured to maintain the spring in a wound-up state.

For some applications, a release interface can be disposed at the proximal end of the driver, and is operatively coupled to the detent in a manner that actuation of the release interface releases the detent, thus triggering the spring to unwind, thereby rotating the drive-head.

For some applications, the detent is a pawl, and the ratchet is part of the winder. For some applications, the detent can be a wire engaged with the drive-head.

For some applications, the amount of potential energy or tension stored (e.g., storable) in the torsion spring can be calibrated for a specific tissue and/or a specific type of tissue anchor, e.g., to ensure full penetration into the tissue without over-tightening. For example, the driver can be configured to limit the extent to which the torsion spring can be wound up, e.g., by including a slip feature.

The method(s)/procedure(s) and steps herein can be performed on a living animal or on a simulation, such as on a cadaver, cadaver heart, simulator (e.g., with the body parts, heart, tissue, etc. being simulated), etc.

In accordance with some applications, a system and/or an apparatus for anchoring in a target tissue of a subject, the system/apparatus including an anchor and a driver. For some applications, the anchor includes an inner helical tissue-engaging element and an outer helical tissue-engaging element. For some applications, the anchor includes a head assembly.

For some applications, the head assembly includes a first head fixed to a proximal end of the inner tissue-engaging element and a second head fixed to a proximal end of the outer tissue-engaging element.

For some applications, the driver reversibly interfaces with and engages the first head and the second head, in order to anchor the system/apparatus in the target tissue.

For some applications, a strength of the anchoring is greater than a system/apparatus having only an outer helical tissue-engaging element or only an inner helical tissue-engaging element.

For some applications, the outer diameter of the inner helical tissue-engaging element is less than the inner diameter of the outer helical tissue-engaging element.

For some applications, the outer helical tissue-engaging element has a tissue-piercing tip at the distal end.

For some applications, the inner helical tissue-engaging element has a tissue-piercing tip at the distal end.

For some applications, at least one of the inner helical tissue-engaging element and the outer tissue-engaging element include a radiopaque substance.

For some applications, at least one of the inner helical tissue-engaging element and the outer helical tissue-engaging element include a polymeric substance.

For some applications, the second head includes an outer cylinder, and the first head includes an independently moveable inner core that interfaces with the second head.

For some applications, the first head and the second head rotate independently.

For some applications, the first head and the second head rotate simultaneously.

For some applications, the first head fits within the second head when both helical tissue-engaging elements are completely inserted into the tissue.

For some applications, the first head and the second head form a locking mechanism when both helical tissue-engaging elements are fully inserted into the target tissue.

For some applications, the driver is configured to advance the inner helical tissue-engaging element into the tissue prior to advancing the outer helical tissue-engaging element into the tissue.

For some applications, the driver is configured to advance the outer helical tissue-engaging element into the tissue prior to advancing the inner helical tissue-engaging element into the tissue.

For some applications, the second head includes a concavity with an opening in a central region of the concavity.

For some applications, the first head fits rotatably within the concavity of the second head.

For some applications, the inner helical tissue-engaging element traverses the opening in the central region of the concavity.

For some applications, the opening includes a set of threads matching a pitch and a handedness of the inner helical tissue-engaging element.

For some applications, turns of the inner helical tissue-engaging element advance into the target tissue by running along the threads.

For some applications, an outer surface of the second head has a non-circular shape.

For some applications, an inner surface of the concavity has a circular shape.

For some applications, the outer surface of the first head has a circular shape.

For some applications, the first head has a non-cylindrical cavity for inserting the driver.

For some applications, the driver includes a first component and a second component.

For some applications, the first component engages the first head, and the second component engages the second head.

For some applications, the first component fits within a cavity of the first head, and the second component fits over the second head.

For some applications, the first component and the second component are independently activatable.

For some applications, the first component and the second component are dependently activatable.

For some applications, the inner helical tissue-engaging element has a first handedness and a first pitch, and the outer helical tissue-engaging element has a second handedness and a second pitch.

For some applications, the first handedness is the same as the second handedness, and the first pitch is the same as the second pitch.

For some applications, the first handedness is opposite the second handedness, and the first pitch is the same as the second pitch.

For some applications, the first handedness is the same as the second handedness, and the first pitch is non-identical to the second pitch.

For some applications, at least one of the first handedness or the first pitch are non-identical to the respective at least one of the second handedness or the second pitch.

For some applications, the first head includes a runner fixed to the inner helical tissue-engaging element.

For some applications, the runner advances along the turns of the outer helical tissue-engaging element to screw the inner helical tissue-engaging element into the target tissue.

For some applications, the runner rides on the turns of the outer helical tissue-engaging element as the driver advances the inner helical tissue-engaging element into the tissue.

For some applications, the runner and the second head form a locking mechanism when both tissue-engaging elements are fully inserted into the target tissue.

For some applications, the runner includes a T-bar.

For some applications, the runner includes a flat disc.

In accordance with some applications, a method (e.g., for anchoring in a target tissue, for anchoring in a target tissue of a subject, etc.) includes transluminally advancing, to a target tissue, a driver that is coupled to a first head and a second head of a head assembly of an anchor.

For some applications, while the driver remains coupled to the first head and the second head, the method includes driving an inner helical tissue-engaging element into the target tissue by using the driver to rotate the first head, and driving an outer helical tissue-engaging element into the target tissue by using the driver to rotate the second head.

For some applications, the method includes, subsequently, decoupling the driver from the head assembly and withdrawing the driver from the subject.

For some applications, rotating the first head turns the inner helical tissue-engaging element.

For some applications, rotating the second head turns the outer helical tissue-engaging element.

For some applications, using the driver to rotate the first head includes using the driver to rotate the first head independently of rotating the second head.

For some applications, driving the inner helical tissue-engaging element into the target tissue includes driving the inner helical tissue-engaging element into the target tissue prior to driving the outer helical tissue-engaging element into the target tissue.

For some applications, driving the inner helical tissue-engaging element into the target tissue includes driving the inner helical tissue-engaging element into the target tissue subsequently to driving the outer helical tissue-engaging element into the target tissue.

For some applications, the first head and the second head rotate simultaneously.

For some applications, the driver first rotates the first head, and subsequently rotates the second head.

For some applications, the driver first rotates the second head, and subsequently rotates the first head.

For some applications, the steps of advancing the inner and outer helical tissue-engaging elements are reversed.

The above method(s)/procedure(s) and steps can be performed on a living animal or on a simulation, such as on a cadaver, cadaver heart, simulator (e.g., with the body parts, heart, tissue, etc. being simulated), etc.

In accordance with some applications, a system and/or an apparatus for anchoring in a target tissue includes an anchor that includes a first head and a helical tissue-engaging element. For some applications, the helical tissue-engaging element is fixed to the first head, and extends away from the first head, distally and helically around an elongate space that is disposed along a longitudinal axis of the anchor.

For some applications, the anchor includes a second head, coupled to the first head in a manner in which the second head is rotatable with respect to the first head and axially fixed with respect to first head.

For some applications, the anchor includes a plurality of prongs fixed to the second head and extending from the second head distally through the elongate space. For some applications, the plurality of prongs are arranged with respect to the helical tissue-engaging element such that rotation of the first head with respect to the second head feeds the prongs laterally outward between progressively proximal turns of the tissue-engaging element.

For some applications, the prongs have elastic properties.

For some applications, the prongs have plastic properties.

For some applications, the first head and the second head are coupled by a snap-fit mechanism.

For some applications, the first head further includes a non-circular protrusion configured to reversibly couple to a driver.

For some applications, turning the first head via the driver fitting into the non-circular protrusion advances the helical tissue-engaging element into the target tissue.

For some applications, the anchor defines a track and a rider, and the second head is coupled to the first head via the rider being slidably coupled to the track.

For some applications, the track is annular.

For some applications, the rider is slidably coupled to the track by protruding into the track.

For some applications, the rider is a first rider of a set of riders, the set of riders being distributed in a ring formation.

For some applications, the track is located on the first head and the rider is located on the second head.

For some applications, the track is located on the second head and the rider is located on the first head.

In accordance with some applications, a method for anchoring in a target tissue includes transluminally advancing, to a target tissue, a driver that is coupled to a first head of a head assembly of an anchor, the first head coupled to a helical tissue-engaging element, and a second head of the head assembly fixed to a plurality of prongs. For some applications, the method includes anchoring the anchor to the tissue by rotating the first head such that the helical tissue-engaging element becomes screwed into the target tissue and the prongs progressively extend laterally outward between progressively proximal turns of the helical tissue-engaging element.

For some applications, the driver rotates the first head by fitting over a non-circular protrusion on the first head.

For some applications, the driver rotates the first head by fitting within a non-circular depression on the first head.

For some applications, anchoring the anchor to the tissue by rotating the first head includes anchoring the anchor to the tissue by rotating the first head such that the second head advances toward the tissue without rotating.

For some applications, the prongs progressively extending laterally outward between progressively proximal turns of the helical tissue-engaging element anchors the anchor in the tissue with a strength of anchoring is greater than a method lacking the prongs progressively extending laterally outward between progressively proximal turns of the helical tissue-engaging element.

The above method(s)/procedure(s) and steps can be performed on a living animal or on a simulation, such as on a cadaver, cadaver heart, simulator (e.g., with the body parts, heart, tissue, etc. being simulated), etc.

In accordance with some applications, a system and/or an apparatus for anchoring in a target tissue includes a primary tissue-engaging element, a head attached to a proximal end of the primary tissue-engaging element, and a set of supplementary tissue-engaging elements coupled to the head. For some applications the set of supplementary tissue-engaging elements are coupled to the head such that, for each supplementary tissue-engaging element of the set, the supplementary tissue-engaging element is constrainable in a disengaged orientation and is biased toward deflecting away from the disengaged orientation and toward an engaged orientation in which the supplementary tissue-engaging element is closer to the primary tissue-engaging element than in the disengaged orientation.

For some applications, each supplementary tissue-engaging element is coupled to the head via an arm.

For some applications, the arm has a double-jointed elbow-type articulation between the supplementary tissue-engaging element and the head.

For some applications, the set includes a pair of supplementary tissue-engaging elements positioned opposite each other around the head.

For some applications, the set includes three supplementary tissue-engaging elements positioned at 120-degree intervals around the head.

For some applications, the set includes four supplementary tissue-engaging elements positioned at 90-degree intervals around the head.

For some applications, the set includes any number of supplementary tissue-engaging elements positioned at equal intervals around the head.

For some applications, each supplementary tissue-engaging element has a hook that penetrates a surface of the target tissue when disposed in its engaged orientation.

In accordance with some applications, a method for anchoring in a real or simulated target tissue of a real or simulated subject includes transluminally advancing, to a target tissue, a driver that is engaged with a head of an anchor, the head fixed to a primary tissue-engaging element, and articulatably coupled to a set of supplementary tissue-engaging elements.

For some applications, the method includes driving the primary tissue-engaging element into the target tissue. For some applications, the method includes, subsequently, withdrawing the driver, such that the supplementary tissue-engaging elements responsively move toward the primary tissue-engaging element and hook into a surface of the tissue.

For some applications, the method further includes pulling proximally on the head such that the supplementary tissue-engaging elements articulate with respect to the head.

For some applications, the method further includes applying a proximal pulling force to the head such that at least some of the proximal pulling force is distributed to the tissue via the supplementary tissue-engaging elements.

The above method(s)/procedure(s) and steps can be performed on a living animal or on a simulation, such as on a cadaver, cadaver heart, simulator (e.g., with the body parts, heart, tissue, etc. being simulated), etc.

In accordance with some applications, a system and/or an apparatus for anchoring in tissue includes an anchor that includes a head, a primary tissue-engaging element, and a supplementary tissue-engaging element. For some applications, the primary tissue-engaging element, extending distally away from the head, thereby defining a longitudinal axis of the anchor and a supplementary tissue-engaging element disposed axially between the head and the primary tissue-engaging element, and protruding radially outward.

For some applications, the supplementary tissue-engaging element protrudes radially outward beyond the head.

For some applications, the supplementary tissue-engaging element comprises a radiopaque material.

For some applications, the helical tissue-engaging element defines the longitudinal axis of the anchor by extending helically around the axis.

For some applications, the supplementary tissue-engaging element is attached to the head at the junction of the head and the primary tissue-engaging element.

For some applications, the anchor defines a gap between the supplementary tissue-engaging element and the primary tissue-engaging element.

For some applications, the supplementary tissue-engaging element is freely rotatable with respect to the primary tissue-engaging element.

For some applications, the supplementary tissue-engaging element is freely rotatable with respect to the head.

For some applications, the supplementary tissue-engaging element includes a ring with protrusions. Each of the protrusions can be a barb that has a point angled distally. For some applications, each barb has a point, and/or upon insertion of the tissue anchor into a target tissue, the points can be angled toward the target tissue.

For some applications, each of the protrusions is a lobe. For some applications, a distal and/or proximal circumferential surface of each lobe is beveled.

For some applications, each lobe has a circumferential directionality that facilitates anchoring of the anchor in the target tissue. For some applications, the circumferential directionality inhibits extrusion of the anchor.

For some applications, the primary tissue-engaging element is configured to be screwed into the target tissue by rotation in a first rotational direction, and/or the circumferential directionality configures the ring to resist rotation within the tissue in a second rotational direction more than in the first rotational direction.

For some applications, the supplementary tissue-engaging element includes a ring having an outer edge, the outer edge foldable within the driver to a diameter not substantially greater than the diameter of the head.

For some applications, the supplementary tissue-engaging element includes a ring having barbs at an outer edge of the ring.

For some applications, the barbs are designed to create friction between the supplementary tissue-engaging element and the target tissue.

For some applications, the supplementary tissue-engaging element and the head collectively define a clutch.

For some applications, the clutch is configured to lock upon the ring being pressed against the head.

In accordance with some applications, a method for anchoring in a real or simulated target tissue includes transluminally advancing, to a target tissue, a driver that is reversibly coupled to a head of an anchor, the head fixed to a primary tissue-engaging element, and a supplementary tissue-engaging element disposed between the head and the primary tissue-engaging element. For some applications, the method also includes anchoring the tissue anchor into the target tissue, such that the supplementary tissue-engaging element engages a surface of the target tissue lateral to the head.

For some applications, anchoring the tissue anchor into the target tissue includes anchoring the tissue anchor into the target tissue such that the supplementary tissue-engaging element provides lateral stabilization to the anchor.

The above method(s)/procedure(s) and steps can be performed on a living animal or on a simulation, such as on a cadaver, cadaver heart, simulator (e.g., with the body parts, heart, tissue, etc. being simulated), etc.

In accordance with some applications, a system and/or an apparatus for verifying a placement of a tissue anchor in a tissue of a subject includes an anchor and a driver. For some applications, the anchor includes a head attached to the tissue-engaging element, the head defining an internal hollow and a port opening into the internal hollow, the internal hollow being open at a tissue-facing surface of the head.

For some applications, the anchor includes a tissue-engaging element. For some applications, the tissue-engaging element is configured to anchor the anchor to the tissue in a manner that places the tissue-facing surface in contact with a surface of the tissue.

For some applications, the driver includes a shaft that defines a channel and is reversibly couplable to the head in a manner that enables the driver to anchor the anchor to the tissue by driving the tissue-engaging element into the tissue and places the channel in fluid communication with the port.

For some applications, the reversible coupling between the driver and the head is configured to engage the driver to position the tissue anchor such that the tissue-facing surface of the head contacts the surface of the tissue.

For some applications, the system/apparatus further includes a dispenser configured to dispense a contrast agent via the channel and the port into the hollow.

For some applications, the dispenser includes a pump.

For some applications, the anchor is configured such that the contact of the tissue-facing surface with the surface of the tissue retains the contrast agent within the hollow.

For some applications, the dispenser includes a capsule containing the contrast agent.

For some applications, the capsule is disposed at a distal portion of the driver.

For some applications, the capsule is disposed at an extracorporeal proximal portion of the driver.

For some applications, the capsule is frangible, and the dispenser is configured to dispense the contrast agent by breaking the capsule.

For some applications, the capsule is compressible, and the dispenser is configured to dispense the contrast agent by compressing the capsule.

In accordance with some applications, a method for verifying a placement of a tissue anchor in a real or simulated tissue of a real or simulated subject includes transluminally advancing, to the tissue, a driver engaged with a head of a tissue anchor, the tissue anchor including a tissue-engaging element fixed to the head, the head defining an internal hollow accessible via a port. For some applications, the method includes, via the engagement between the driver and the head, driving the tissue-engaging element into the tissue. For some applications, the method includes, subsequently, via a channel of the driver that is in fluid communication with the port, dispensing a contrast agent into the internal hollow.

For some applications, the internal hollow is open at a tissue-facing surface of the head, and driving the tissue-engaging element into the tissue includes driving the tissue-engaging element into the tissue such that the tissue-facing surface contacts a surface of the tissue.

For some applications, dispensing the contrast agent into the internal hollow includes dispensing the contrast agent into the internal hollow by piercing a capsule containing the contrast agent, the capsule held in the channel until the contrast agent is dispensed.

For some applications, dispensing the contrast agent into the internal hollow includes dispensing the contrast agent into the internal hollow by compressing a capsule containing the contrast agent, the capsule held in the channel until the contrast agent is dispensed.

For some applications, anchoring the tissue-engaging element into the tissue includes anchoring the tissue anchor into the tissue such that the tissue-facing surface of the head contacts a surface of the tissue.

For some applications, the method further includes radiographically imaging the anchor and the contrast agent.

For some applications, radiographically imaging the anchor and the contrast agent includes radiographically imaging the anchor and the contrast agent to check for the presence of the contrast agent in the hollow of the tissue anchor head.

For some applications, radiographically imaging the anchor and the contrast agent includes radiographically imaging the anchor and the contrast agent within a predetermined amount of time after dispensing the contrast agent.

For some applications, when the tissue-facing surface of the head contacts the surface of the tissue, the contrast agent is retained in the hollow.

For some applications, anchoring the tissue-engaging element into the tissue includes anchoring the tissue anchor into the tissue such that the tissue-facing surface of the head fails to contact a surface of the tissue.

For some applications, when the tissue-facing surface of the head fails to contact the surface of the tissue, contrast agent dispensed into the hollow through the port on the upper surface diffuses out of the hollow essentially as it is being dispensed.

The above method(s)/procedure(s) and steps can be performed on a living animal or on a simulation, such as on a cadaver, cadaver heart, simulator (e.g., with the body parts, heart, tissue, etc. being simulated), etc.

In accordance with some applications, a system and/or an apparatus for anchoring in a target tissue, the system/apparatus including an anchor and a driver. For some applications, the anchor includes an outer helical tissue-engaging element and an inner helical tissue-engaging element disposed coaxially within the outer helical tissue-engaging element.

For some applications, the anchor includes a head assembly fixed to a proximal end of the inner tissue-engaging element and to a proximal end of the outer tissue-engaging element. For some applications, the driver is reversibly engageable with the head assembly, such that rotation of the head assembly by the driver concurrently rotates both tissue-engaging elements.

In accordance with some applications, a system and/or an apparatus for anchoring to a tissue includes an anchor and a driver. For some applications, the anchor can include a helical tissue-engaging element, and a head. For some applications, the head can include an interface, and/or a torque limiter, operatively coupling the interface to the tissue-engaging element.

For some applications, the driver can be reversibly engageable with the interface, and/or configured, while engaged with the interface, to screw the tissue-engaging element into the tissue by applying torque to the interface. For some applications, the torque limiter can be configured to transfer the torque to the tissue-engaging element, only up to a predefined threshold torque.

For some applications, the torque limiter includes a slip clutch.

For some applications, the torque limiter includes a shear pin.

For some applications, the torque limiter is a ball-detent torque limiter.

For some applications, the torque limiter includes a gear set.

For some applications, the torque limiter is disposed axially between the interface and the tissue-engaging element.

For some applications, the torque limiter limits the torque transferred to the tissue-engaging element by slipping upon the torque exceeding the predefined threshold torque.

For some applications, the torque limiter limits the torque transferred to the tissue-engaging element by operatively uncoupling the interface from the tissue-engaging element upon the torque exceeding the predefined threshold torque.

For some applications, the driver is configured to unscrew the tissue-engaging element from the tissue by applying reverse torque to the interface, and

the torque limiter is configured to transfer the reverse torque to the tissue-engaging element even upon the reverse torque exceeding the predefined threshold torque.

For some applications, the torque limiter is a pawl-and-spring torque limiter including a drive pawl, a series of notches, and a spring configured to hold the drive pawl against the series of notches.

For some applications, the spring is fixed to the interface.

For some applications, the spring is two springs, the first spring oriented to limit torque in a direction of rotation, and the second spring oriented to limit torque in an opposite direction of rotation.

For some applications, the head further includes a base, the base defining a tissue-facing surface, fixed with respect to the tissue-engaging element, and defining the series of notches.

For some applications, the spring is disposed inside the base.

For some applications, the spring is a spiral spring that defines a spring plane, the series of notches lying on the spring plane.

For some applications, the series of notches is arranged to circumscribe the spiral spring.

For some applications, the spring plane is parallel to the tissue-facing surface.

For some applications, the spiral spring is shaped to define a double spiral having two spiral arms.

In accordance with some applications, a method for anchoring to a real or simulated tissue of a real or simulated subject includes transluminally advancing, to the tissue, a driver engaged with an interface of an anchor, the anchor including a helical tissue-engaging element, and a torque limiter operatively coupling the interface to the tissue-engaging element.

For some applications, the method can include, using the driver, screwing the tissue-engaging element into the tissue by applying torque to the interface such that the torque limiter transfers the torque to the tissue-engaging element until the torque exceeds a predefined threshold torque; and the torque limiter responsively ceases to transfer the torque to the tissue-engaging element.

For some applications, the method further includes, subsequently to screwing the tissue-engaging element into the tissue, disengaging the driver from the interface and withdrawing the driver from the subject.

For some applications, the method further includes, responsively to the torque limiter ceasing to transfer the torque to the tissue-engaging element, unscrewing the tissue-engaging element from the tissue by applying reverse torque to the interface using the driver.

In accordance with some applications, a system and/or an apparatus for anchoring to a tissue of a subject includes an anchor including a head, including an interface; a tissue-engaging element; and/or a flexible sleeve. For some applications, the tissue-engaging element and the flexible sleeve can extend distally away from the head.

For some applications, the sleeve includes a fabric. For some applications, the sleeve includes a mesh. For some applications, the sleeve includes a braid. For some applications, the sleeve includes a flexible film.

For some applications, the sleeve includes a metal. For some applications, the sleeve includes a polymer.

For some applications, a stiffness of a distal part of the sleeve is greater than a stiffness of a proximal part of the sleeve.

For some applications, a stiffness of a proximal part of the sleeve is greater than a stiffness of a distal part of the sleeve.

For some applications, the sleeve includes a porous sheet.

For some applications, the porous sheet includes pores having a pore-size greater than 75 microns.

For some applications, the system/apparatus further includes a driver, configured to reversibly engage the interface and to drive the tissue-engaging element into the tissue.

For some applications, the driver is configured to drive the tissue-engaging element into the tissue in a manner that axially compresses the sleeve between the head and a surface of the tissue.

For some applications, the sleeve is configured to facilitate an amount of axial compression of the sleeve, and to resist compression of the sleeve beyond the amount.

For some applications, the tissue-engaging element is configured to be driven sufficiently deep into the tissue that the sleeve becomes axially compressed by the amount.

For some applications, the sleeve is configured to spread outward on the surface of the tissue upon being axially compressed between the head and the surface of the tissue.

For some applications, the sleeve defines a lumen, the tissue-engaging element extends through the lumen, and the anchor is configured such that, the tissue-engaging element progressively exits a distal end of the lumen upon being progressively driven into the tissue.

For some applications, the driver is configured to drive the tissue-engaging element into the tissue in a manner that the sleeve remains extending distally away from the head.

For some applications, the tissue-engaging element includes a threaded shaft having a sharp distal tip.

For some applications, the system/apparatus further includes a ring attached to a distal end of the flexible sleeve, and threadedly engaged with the threaded shaft.

For some applications, the tissue-engaging element is configured to be driven linearly into the tissue.

For some applications, the ring is threadedly engaged with the shaft in a manner that maintains the sleeve extending distally away from the head while the tissue-engaging element is driven linearly into the tissue.

For some applications, the driver is configured to rotate the threaded shaft with respect to the ring.

For some applications, the driver is configured to rotate the threaded shaft with respect to the ring in a manner that draws the ring toward the head, and the sleeve is configured to spread radially outward within the tissue, responsively to the ring being drawn toward the head.

For some applications, the tissue-engaging element defines a central longitudinal axis of the anchor, and the sleeve is compressible along the central longitudinal axis.

For some applications, the sleeve is compressible such that, as the sleeve becomes compressed, the sleeve spreads radially outward from the central longitudinal axis.

For some applications, the tissue-engaging element extends through a lumen of the sleeve.

For some applications, the sleeve substantially covers the tissue-engaging element.

For some applications, the system/apparatus further includes prongs, coupled to the head, and extending, within the lumen, distally away from the head.

For some applications, the prongs are covered by the sleeve.

For some applications, the prongs include barbs.

For some applications, the prongs include spikes.

For some applications, the prongs include metal wires.

For some applications, the prongs are disposed laterally from the tissue-engaging element.

For some applications, the prongs are dimensioned to become exposed from the sleeve upon the sleeve becoming compressed along the central longitudinal axis.

For some applications, the anchor includes a collar via which the prongs are coupled to the head.

For some applications, the collar is disposed axially between the head and the tissue-engaging element.

For some applications, the collar is rotatably coupled to the head.

For some applications, the sleeve further includes a flexible tubular sheet, and a flexible wire extending helically along the sheet.

For some applications, the sheet includes a fabric.

For some applications, the wire is configured to support the sheet being tubular, but is compressible.

For some applications, the wire is woven into the sheet.

For some applications, the wire is embedded in the sheet.

For some applications, the wire is formed from a metal.

For some applications, the wire is formed from a polymer.

For some applications, the wire is echogenic.

For some applications, the wire is radiopaque.

For some applications, the sleeve has a length along which the sleeve extends distally away from the head, and the wire extends helically along the sheet for the entire length of the sleeve.

In accordance with some applications, a method for anchoring to a real or simulated tissue of a real or simulated subject includes transluminally advancing, to the tissue, a driver reversibly coupled to an anchor, the anchor including a head, a compressible sleeve, and a helical tissue-engaging element.

For some applications, the method can further include, using the driver, screwing the tissue-engaging element into the tissue, such that the sleeve becomes compressed on a surface of the tissue as the tissue-engaging element advances into the tissue; and when the tissue-engaging element is completely anchored in the tissue, the sleeve is disposed between the head and a surface of the tissue.

For some applications, screwing the tissue-engaging element into the tissue includes screwing the tissue-engaging element into the tissue such that a set of short prongs, coupled to the head, become driven into the tissue.

For some applications, the sleeve being compressed on the surface of the tissue further includes the sleeve spreading outward from a central longitudinal axis of the anchor.

For some applications, the sleeve being compressed on the surface of the tissue is enhanced by the sleeve including a helical wire.

For some applications, the compression of the on the surface of the tissue is enhanced by a stiffness of a proximal part of the sleeve being different than a stiffness of a distal part of the sleeve.

In accordance with some applications, a system and/or an apparatus for anchoring to a tissue includes a driver and an anchor. For some applications, the anchor can include a head, a first helical tissue-engaging element, and a second helical tissue-engaging element.

For some applications, the first helical tissue-engaging element can have a fixed diameter not substantially greater than a diameter of the head. For some applications, the second helical tissue-engaging element can have a pre-deployment diameter not substantially greater than the diameter of the head, and a post-deployment diameter substantially greater than the diameter of the head. For some applications, deployment of the anchor can result in the second tissue-engaging anchor assuming its post-deployment diameter.

For some applications, the driver is configured to drive the anchor out of the delivery tool such that the second helical tissue-engaging element expands radially outward from the first helical tissue-engaging element.

For some applications, a strength of the anchoring is greater than a system/apparatus having only a single helical tissue-engaging element.

For some applications, the first helical tissue-engaging element includes stainless steel.

For some applications, the first helical tissue-engaging element includes a shape memory material.

For some applications, the second helical tissue-engaging element includes a shape memory material.

For some applications, the shape memory material is nitinol.

For some applications, the system/apparatus further includes a removable capsule surrounding and constraining the first helical tissue-engaging element and the second helical tissue-engaging element pre-deployment.

For some applications, the removable capsule is coupled to the driver.

For some applications, the removable capsule defines internal threads which at least one of the first tissue-engaging element and the second tissue-engaging element traverse during deployment.

For some applications, the internal threads are present throughout the length of the removable capsule.

In accordance with some applications, a method for anchoring to a real or

simulated tissue of a real or simulated subject includes transluminally advancing, to the tissue, a driver, and an anchor including a head, a first helical tissue-engaging element, and a second helical tissue-engaging element.

For some applications, the method can further include screwing the anchor into the tissue by using the driver to screw the anchor, such that as the anchor advances into the tissue, the second helical tissue-engaging element assumes a preset memory shape having a diameter greater than the diameter of the first helical tissue-engaging element.

For some applications, the method further includes, subsequently to screwing the tissue-engaging element into the tissue, disengaging the driver from the anchor and withdrawing the driver from the subject.

For some applications, the method further includes transluminally advancing the driver and the anchor while the anchor is disposed within a removable capsule of the driver, the capsule having internal threads. Using the driver to screw the anchor into the tissue, includes using the driver to screw the anchor out of the removable capsule along the internal threads and into the tissue.

In accordance with some applications, a system and/or an apparatus for anchoring to a tissue includes an anchor including a tissue-engaging element and a head. For some applications, the head can have a deployed width greater than an undeployed width.

For some applications, the system/apparatus can further include a delivery tool. For some applications, the delivery tool can include a tube, having an internal diameter that is smaller than the deployed width of the head, and a driver, configured to drive the anchor out of the tube such that the head expands toward the deployed width.

For some applications, the head includes a shape memory substance.

For some applications, a strength of the anchoring is greater than an anchor having the deployed width of the head less than the inner diameter of the tube.

For some applications, the tissue-engaging element has a tissue-piercing tip at a distal end.

For some applications, the anchor includes a continuous wire that is shaped to define both the head and the tissue-engaging element.

For some applications, a deployed shape of the head includes a coil.

For some applications, the coil defines a planar spiral.

For some applications, deployment of the anchor into the tissue is accompanied by the head expanding toward the deployed shape.

For some applications, a deployed width of the tissue-engaging element is greater than the internal diameter of the tube.

For some applications, deployment of the anchor into the tissue is accompanied by the tissue-engaging element expanding toward the deployed width.

For some applications, the tissue-engaging element includes a shape memory substance.

For some applications, a strength of the anchoring is greater than an anchor having the deployed width of the tissue-engaging element smaller than the inner diameter of the tube.

For some applications, the anchor is held in an elongated state.

For some applications, a width of the elongated state is less than the inner diameter of the tube.

For some applications, a strength of the anchoring is greater than an anchor having a deployed width less than the inner diameter of the tube.

For some applications, the elongated state of the anchor holds the anchor under tension.

For some applications, deployment of the anchor by the driver releases the tension, enabling the tissue-engaging element to advance into the tissue.

For some applications, deployment of the anchor by the driver enables the tissue-engaging element to expand toward the deployed width.

For some applications, the anchor is withdrawable by pulling tension.

For some applications, the anchor includes a length of memory shape wire.

For some applications, the anchor has a final shape set that mimics a one-piece helical tissue-engaging element and head.

For some applications, the head includes a crossbar configured to be grasped by a discrete retrieval tool.

For some applications, the anchor is a component of an implant, and the crossbar is configured to be coupled to another component of the implant.

In accordance with some applications, a method for anchoring to a real or simulated tissue of a real or simulated subject includes transluminally advancing, to the tissue, a delivery tool including a tube and a driver, and an anchor constrained linearly within the delivery tool.

For some applications, the method can further include driving the anchor into the tissue by using the driver to deploy the anchor such that, as the anchor advances into the tissue, the anchor forms a tissue-engaging element within the tissue by assuming a preset memory shape. For some applications, the method can further include subsequently withdrawing the tube from the anchor and withdrawing the delivery tool from the subject.

For some applications, deploying the anchor includes deploying the anchor such that the anchor assumes a diameter greater than an inner diameter of the tube.

For some applications, the preset memory shape has a diameter greater than an inner diameter of the tube, and driving the anchor into the tissue includes driving the anchor into the tissue such that the tissue-engaging element assumes the preset memory shape that has the diameter greater than the inner diameter of the tube.

For some applications, withdrawing the tube from the anchor includes withdrawing the tube from the anchor such that the anchor forms a head at a surface of the tissue.

For some applications, withdrawing the tube from the anchor includes withdrawing the tube from the anchor such that the anchor forms the head to have a diameter greater than an inner diameter of the tube.

For some applications, the anchor has a head, and withdrawing the tube from the anchor includes withdrawing the tube from the anchor such that the head expands to have a diameter greater than an inner diameter of the driver.

For some applications, deploying the anchor removes the linear constraint, enabling the tissue-engaging element to advance into the tissue.

For some applications, driving the anchor into the tissue releases the linear constraint.

For some applications, the anchor assuming the preset memory shape includes the head expanding toward a deployed shape.

For some applications, the method further includes checking a position of the anchor, and withdrawing the anchor from the tissue by pulling tension if the position requires adjustment.

For some applications, withdrawing the anchor from the tissue by pulling tension is accomplished by pulling on a proximal end of the head, the proximal end including a crossbar.

In accordance with some applications, a system and/or an apparatus for anchoring to a tissue can include a delivery tool, including a driver, and a sheath having an inner helical thread and a set of inner axial grooves; and an anchor. For some applications, the anchor can be disposed within the sheath.

For some applications, the anchor can include a head, including an interface; a helical tissue-engaging element, extending distally away from the head, and having a size, pitch, and handedness complementary to the helical thread; and a second tissue-engaging element, including a set of prongs.

For some applications, the set of prongs can extend distally away from the head, and can be disposed laterally from the helical tissue-engaging element. For some applications, the prongs can be sized and positioned complementarily to the axial grooves, and rotatably coupled to the helical tissue-engaging element.

For some applications, the driver can be configured to drive the anchor distally out of the sheath by rotating the head such that the helical tissue-engaging element advances helically along the helical thread, and each of the prongs advances axially along a respective one of the axial grooves.

For some applications, the sheath is configured such that the axial grooves intersect the helical groove.

For some applications, the prongs include barbs.

For some applications, the prongs include spikes.

For some applications, the prongs include metal wires.

For some applications, the prongs have a preset memory shape, such that each prong is biased to extend laterally in a shape of a proximally-directed hook when released from the sheath.

For some applications, the sheath defines a longitudinal slit extending a length of the sheath.

For some applications, the anchor includes a collar via which the prongs are coupled to the head.

For some applications, the collar is disposed axially between the head and the tissue-engaging element.

For some applications, the collar is rotatably coupled to the head.

For some applications, the collar and the prongs are formed from a single unitary component that is shaped to define the collar and the prongs.

For some applications, the collar and the prongs are formed by additive manufacturing.

For some applications, the collar and the prongs are formed as separate components and subsequently attached to each other.

For some applications, the helical thread inhibits expansion of the second tissue-engaging element.

In accordance with some applications, a method for anchoring to a real or simulated tissue of a real or simulated subject includes: transluminally advancing, to the tissue, a driver; an anchor including a head, a helical tissue-engaging element, and a second tissue-engaging element including a set of prongs; and an anchor sheath unsheathing the anchor.

For some applications, the method can further include driving the anchor into the tissue by using the driver to deploy the anchor, such that as the anchor advances into the tissue, the prongs assume a preset memory shape having a diameter greater than a diameter of the anchor sheath.

For some applications, the method further includes, subsequently to driving the first tissue-engaging element into the tissue, disengaging the driver from the anchor and withdrawing the driver from the subject.

In accordance with some applications, a system and/or an apparatus for anchoring to a tissue includes an anchor, having a longitudinal axis. For some applications, the anchor can include a head, defining, on the longitudinal axis, an aperture therethrough; and a set of tissue-engaging prongs, arranged circumferentially around the longitudinal axis.

For some applications, each prong can define a pocket, and be biased to extend laterally from the longitudinal axis. For some applications, the anchor can have an anchoring state in which the prongs extend laterally from the longitudinal axis. For some applications, the anchor can further include an insert that includes a set of fingers, corresponding to the set of tissue-engaging prongs.

For some applications, the insert can be dimensioned to be secured to the anchor by extending through the aperture such that the fingers are disposed within the pockets in a manner that constrains the anchor in a delivery state in which the prongs are substantially parallel with the longitudinal axis.

For some applications, the insert is configured to be intracorporeally retracted through the aperture in a manner that withdraws the fingers from the pockets.

For some applications, the insert includes a radiopaque material.

For some applications, the anchor includes a radiopaque material.

For some applications, in the anchoring state, the insert is configured to be completely withdrawn from the anchor.

For some applications, in the anchoring state, the insert is configured to be partially retained within the anchor, obstructing the aperture.

For some applications, the pockets face medially in the delivery state.

For some applications, the fingers are parallel with the longitudinal axis.

For some applications, the fingers are parallel with each other.

For some applications, the tissue-engaging prongs include a shape memory material.

For some applications, the shape memory material is nitinol.

For some applications, the insert includes a shaft having a proximal head and a sharp distal tip.

For some applications, in the delivery state, the distal tip is configured to extend beyond the prongs.

For some applications, the distal tip is configured to serve as a lance.

For some applications, the insert is configured to be partially retracted such that the distal tip is configured to remain within the anchor in the anchoring state.

In accordance with some applications, a method for anchoring to a real or simulated tissue of a real or simulated subject includes introducing, into the subject, an anchor, in a delivery state thereof, the anchor including a head, and a set of prongs coupled to the head, and an insert, disposed through an aperture defined by the head, and constraining the anchor in the delivery state by fingers of the insert being disposed within corresponding pockets defined by the prongs.

For some applications, the method can further include, while the anchor remains in the delivery state, driving the prongs into the tissue; and subsequently, withdrawing the fingers from the pockets by retracting the insert through the aperture such that the anchor transitions toward an anchoring state in which the tissue-engaging prongs extend laterally from the longitudinal axis.

For some applications, retracting the insert through the aperture includes completely removing the insert from the anchor.

For some applications, retracting the insert through the aperture includes withdrawing the fingers from the pockets such that the insert remains disposed through the aperture.

In accordance with some applications, a system and/or an apparatus for anchoring to a tissue includes an anchor that includes a head; and a tissue-engaging element that includes a shaft having a lateral wall, and a pointed tip configured to facilitate insertion of the lateral surface into the tissue.

For some applications, the system/apparatus can further include a balloon, inflatable to expand radially away from the lateral wall within the tissue.

For some applications, the shaft of the tissue-engaging element is hollow.

For some applications, the shaft of the tissue-engaging element includes a sharp distal tip configured to facilitate entry of the tissue-engaging element into the tissue.

For some applications, the balloon occupies substantially the entire shaft of the tissue-engaging element.

For some applications, the shaft includes a series of windows via which the balloon is configured to expand radially away from the lateral wall.

For some applications, the series of windows includes exactly two windows. For some applications, the series of windows includes exactly three windows. For some applications, the series of windows includes exactly four windows. For some applications, the series of windows includes exactly five windows. For some applications, the series of windows includes exactly six windows.

For some applications, the head defines an aperture that provides fluid communication with the balloon.

For some applications, the aperture is fitted with a closure.

For some applications, the closure serves as a one-way valve.

For some applications, the system/apparatus further includes a balloon expander.

For some applications, the balloon expander is in fluid communication with the balloon via the closure.

For some applications, the balloon expander contains a fluid.

For some applications, the balloon expander is configured to inflate the balloon by injecting the fluid via the closure.

For some applications, the fluid is saline.

For some applications, the fluid includes a resin.

For some applications, the resin is an epoxy resin.

For some applications, the resin is configured to harden by exposure to UV light.

For some applications, the system/apparatus further includes an optical fiber, coupled to the balloon expander, and configured to transmit UV light to the balloon.

For some applications, the system/apparatus further includes a delivery tool, configured to percutaneously advance the anchor to the tissue.

For some applications, the delivery tool includes an optical fiber configured to transmit UV light to the balloon.

For some applications, the delivery tool is configured to insert the tissue-engaging element into the tissue.

For some applications, the delivery tool includes a balloon expander, the delivery tool being couplable to the anchor in a manner in which the balloon expander is in fluid communication with the balloon.

For some applications, the balloon expander contains a fluid, and is configured to inflate the balloon by injecting the fluid into the balloon.

For some applications, the fluid is saline.

For some applications, the fluid includes a resin.

For some applications, the resin is an epoxy resin.

For some applications, the resin is configured to harden by exposure to UV light.

In accordance with some applications, a method for anchoring to a real or simulated tissue of a real or simulated subject includes transluminally advancing, to the tissue, an anchor that includes a head, and a tissue-engaging element that includes a shaft and a balloon; using a driver, driving the tissue-engaging element into the tissue; and subsequently, inflating the balloon within the tissue by injecting a fluid thereinto, such that the balloon expands radially away from the shaft within the tissue.

For some applications, injecting the fluid includes injecting the fluid via a one-way valve in the shaft.

For some applications, driving the tissue-engaging element into the tissue includes driving the tissue-engaging element distally into the tissue with a sharp distal tip of the shaft penetrating into the tissue.

For some applications, inflating the balloon such that the balloon expands radially away from the shaft, includes inflating the balloon such that the balloon expands radially through a set of windows in the shaft.

For some applications, injecting the fluid includes injecting a polymeric substance.

For some applications, the method further includes, subsequently to injecting the polymeric substance, hardening the polymeric substance by exposing the polymeric substance to UV light.

For some applications, exposing the polymeric substance to UV light includes transmitting the UV light via an optical fiber included in the driver.

In accordance with some applications, a system and/or an apparatus for anchoring to a tissue includes an anchor, including a cup defining a cavity, and having a flexible flange that defines a rim of the cup, a head having a port opening into the cavity; and a delivery tool, configured to percutaneously advance the anchor to the tissue, place the flexible flange against the tissue, and while the flexible flange remains against the tissue, anchor the anchor to the tissue by applying suction to the cup via the port.

For some applications, the port includes a check valve.

For some applications, the tissue is tissue of an annulus of a valve of a heart of the subject, and the delivery tool is configured to place the flexible flange against the tissue of the annulus, and while the flexible flange remains against the tissue of the annulus, anchor the anchor to the annulus of the valve by applying suction to the cup via the port.

For some applications, the flange is configured to seal against the tissue.

For some applications, the flange includes soft silicone molded over an outside rim of the cup.

For some applications, the cup is less flexible than the flange.

For some applications, the cup includes a stiff material.

For some applications, the stiff material is a metal.

For some applications, the stiff material is a plastic.

For some applications, the port includes a valve.

For some applications, the valve includes a valve member that includes a stopper that secures the valve member in place.

In accordance with some applications, a method for anchoring to a real or simulated tissue of a real or simulated subject includes transluminally advancing, to the tissue, an anchor that includes: a cup defining a cavity and having a flexible flange, and a head having a port that opens into the cavity; and using a delivery tool, placing the flexible flange against the tissue, and while the flexible flange remains against the tissue, anchoring the anchor to the tissue by applying suction to the cup via the port.

For some applications, applying suction to the cup includes applying suction to the cup in a manner that seals the flange to the tissue.

For some applications, applying suction to the cup includes sucking fluid out of the cup via the port.

For some applications, the tissue is tissue of the heart, and transluminally advancing includes transluminally advancing to the tissue of the heart.

For some applications, the tissue is tissue of an annulus of a valve of the heart, and anchoring the anchor to the tissue includes anchoring the anchor to the annulus.

In accordance with some applications, a system and/or an apparatus for verifying a placement of a tissue anchor in a tissue includes an anchor, including a head; a tissue-engaging element having a sharpened distal tip, and configured to be driven into the tissue; and a ring, slidably coupled to the helical tissue-engaging element, such that, responsively to driving of the helical tissue-engaging element into the tissue, the ring slides along tissue-engaging element toward the head.

For some applications, the ring includes a radiopaque material.

For some applications, the ring includes an echogenic material.

For some applications, the ring is configured to resist entering the tissue as the driver advances the tissue-engaging element into the tissue.

For some applications, the tissue-engaging element is a helical tissue-engaging element configured to be screwed into the tissue.

For some applications, the system/apparatus further includes a driver configured to advance the helical tissue-engaging element into the tissue.

For some applications, the ring is configured to slide proximally along progressive turns of the helical tissue-engaging element as the driver advances the tissue-engaging element into the tissue.

In accordance with some applications, a method for verifying a placement of a tissue anchor in a real or simulated tissue of a real or simulated subject includes transluminally advancing, to the tissue, a driver engaged with a head of an anchor, the anchor further including a tissue-engaging element, extending distally from the head, and having a sharpened distal tip, and a ring slidably coupled to the tissue-engaging element, driving the tissue-engaging element into the tissue; and fluoroscopically viewing the anchor to determine a position of the ring along the tissue-engaging element.

For some applications, the tissue-engaging element is a helical tissue-engaging element, and driving the tissue-engaging element into the tissue includes screwing the tissue-engaging element into the tissue.

For some applications, the method further includes, responsively to determining the position of the ring along the tissue-engaging element, driving the tissue-engaging element further into the tissue.

For some applications, the method further includes, responsively to determining the position of the ring along the tissue-engaging element, disengaging the driver from the head of the anchor.

In accordance with some applications, a system and/or an apparatus for use with a tissue of a subject includes an anchor, including a head, and a helical tissue-engaging element extending distally away from the head.

For some applications, the anchor can further include an indicator wire, having a tip, and a discrete bending location or site at which the indicator wire is biased to form an acute bend.

For some applications, the system/apparatus can further include a delivery tool, including a driver that is engaged with the anchor. For some applications, the delivery tool can be coupled to the indicator wire in a manner that defines a delivery state of the system/apparatus, in which the delivery tool is configured to advance the anchor to the tissue while the indicator wire is constrained such that the tip is disposed distally from the bending site.

For some applications, the delivery tool can be configured to progressively drive the tissue-engaging element into the tissue in a manner that progressively feeds the indicator wire out of the delivery tool.

For some applications, the wire can be configured such that, in response to the driver achieving a predefined amount of driving of the anchor into the tissue, the wire abruptly bends to form the acute bend at the discrete bending location.

For some applications, the wire is configured such that the predefined amount corresponds to the bending site reaching a slit in a lateral wall of the delivery tool.

For some applications, the wire is configured such that the predefined amount corresponds to the bending site reaching a distal rim of the delivery tool.

For some applications, the wire is configured such that the predefined amount corresponds to the helical tissue-engaging element being screwed fully into the tissue.

For some applications, the distal tip is radiopaque.

For some applications, the indicator wire is attached to the head.

For some applications, the indicator wire is attached to the driver.

For some applications, the wire is configured such that, in response to the driver achieving the predefined amount of driving of the anchor into the tissue, the distal tip abruptly springs proximally by the wire forming the acute bend at the discrete bending site or location.

For some applications, the delivery tool further includes a delivery capsule, the delivery tool configured to percutaneously deliver the anchor to the tissue while the anchor is disposed within the delivery capsule.

For some applications, the indicator wire is attached to the delivery capsule.

For some applications, the delivery capsule has a lateral wall that defines a longitudinal slit therein.

For some applications, the indicator wire is configured to exit the delivery capsule via the longitudinal slit.

In accordance with some applications, a method for verifying a placement of an anchor in a real or simulated tissue of a real or simulated subject includes using a delivery tool, transluminally advancing an anchor to the tissue, the anchor including a head and a tissue-engaging element extending distally away from the head. For some applications, the method includes using a driver of the delivery tool, engaged with the head of the anchor, progressively driving the tissue-engaging element into the tissue in a manner that progressively feeds an indicator wire out of the delivery tool, such that, upon achieving a predetermined amount of driving, the indicator wire abruptly bends at a predefined discrete bending site of the wire.

For some applications, the indicator wire includes a radiopaque distal tip, and progressively driving the tissue-engaging element into the tissue includes progressively driving the tissue-engaging element into the tissue such that, upon achieving the predetermined amount of driving, the distal tip springs proximally.

For some applications, the tissue-engaging element is a helical tissue-engaging element, and driving the tissue-engaging element into the tissue includes screwing the tissue-engaging element into the tissue.

For some applications, the method further includes fluoroscopically determining a position of the indicator wire.

For some applications, the method further includes, responsively to determining the position of the indicator wire, driving the tissue-engaging element further into the tissue.

For some applications, the method further includes, responsively to determining the position of the indicator wire, disengaging the driver from the anchor.

In accordance with some applications, a system and/or an apparatus for use with tissue includes an anchor head, and/or a tissue-engaging element, fixed to the anchor head. For some applications, the system/apparatus can further comprise a prong, having a resting state in which the prong is shaped to define a hook.

For some applications, the system/apparatus can include a driver, a distal end of which is transluminally advanceable to the tissue while engaged with the anchor head. For some applications, the driver can be configured to drive the prong into the tissue such that the prong curves to form the hook within the tissue, and/or the tissue-engaging element into the tissue, facilitated by a counterforce provided via the prong in the tissue.

For some applications, the system/apparatus includes an anchor, the anchor including the anchor head, the tissue-engaging element, and/or the prong.

For some applications, the system/apparatus includes a delivery tool, the delivery tool including the driver and/or the prong.

For some applications, the prong is one of a set of prongs, and/or the prongs of the set are distributed around the anchor head. For some applications, the prong is one of a set of prongs, and/or the set includes two prongs positioned opposite each other around the anchor head.

For some applications, the prong is one of a set of prongs, and/or the set includes three prongs positioned at 120-degree intervals around the anchor head. For some applications, the prong is one of a set of prongs, and/or the set includes four prongs positioned at 90-degree intervals around the anchor head.

For some applications, the system/apparatus is sterilized.

For some applications, the prong includes a shape-memory alloy.

For some applications, the prong includes a radiopaque material.

For some applications, the anchor head is configured to limit lateral movement of the prong. For some applications, the anchor head is configured to constrain the prong within the tissue.

For some applications, the anchor head defines a hole, and/or the driver is configured to drive the prong into the tissue through the hole. For some applications, the driver is configured to remove the prong from the tissue by retracting the prong proximally through the hole.

In accordance with some applications, a method for anchoring in a real or simulated tissue includes transluminally advancing, to the tissue a distal end of a driver, and/or engaged by the distal end of the driver, an anchor head fixed to a tissue-engaging element. For some applications, the method can further include using the driver, driving a prong into the tissue such that the prong curves to form a hook within the tissue, and/or using the driver, driving the tissue-engaging element into the tissue, facilitated by a counterforce provided via the prong in the tissue.

For some applications, the method further includes sterilizing the prong.

For some applications, the prong includes a radiopaque material, and/or the method further includes imaging the radiopaque prong in the tissue.

For some applications, driving the tissue-engaging element into the tissue includes screwing the tissue-engaging element into the tissue.

For some applications, the method further includes sterilizing the driver. For some applications, the method further includes sterilizing the anchor head fixed to the tissue-engaging element.

For some applications, the method further includes disengaging the anchor head from the driver, and/or withdrawing the driver.

For some applications, the method further includes removing the prong from the tissue while leaving the tissue-engaging element within the tissue.

For some applications, the method further includes disengaging the anchor head from the driver, and/or withdrawing the driver, leaving the tissue-engaging element and/or the prong within the tissue.

For some applications, the prong includes a shape-memory alloy, and driving the prong into the tissue includes driving the prong into the tissue such that the prong curves to form the hook by the shape-memory alloy assuming a memory shape thereof.

For some applications, the prong is a first prong of a set of prongs, and/or the method includes driving the set of prongs into the tissue.

For some applications, the counterforce is provided by applying a proximal pulling force to the prong, and/or driving the tissue-engaging element into the tissue, facilitated by the counterforce includes driving the tissue-engaging element into the tissue, is facilitated by the counterforce that is provided by the proximal pulling force applied to the prong.

For some applications, the prong includes a set of prongs, and applying the proximal pulling force to the prong includes applying a proximal pulling force to the set of prongs, such that the prongs engage the tissue.

For some applications, applying the proximal pulling force to the set of prongs is applied in a manner that pulls a surface of the tissue toward the driver while driving the tissue-engaging element into the tissue.

For some applications, the set of prongs are shape-memory prongs, and driving the supplemental tissue-engaging element into the tissue includes driving the shape-memory prongs into the tissue, such that the prongs assume their memory shape within the tissue.

In accordance with some applications, a system and/or an apparatus for anchoring an anchor to tissue includes a driver that includes a drive-head at a distal end of the driver, the drive-head configured to be transluminally advanced to the tissue while engaged with the anchor.

For some applications, the system/apparatus can further comprise a torsion spring and/or a winder at a proximal portion of the driver. For some applications, the winder can be operatively coupled to the torsion spring such that operation of the winder winds up the spring.

For some applications, the system/apparatus can further comprise a detent, configured to maintain the spring wound-up, and/or a release interface at a proximal end of the driver, operatively coupled to the detent such that actuation of the release interface triggers the spring to unwind in a manner that rotates the drive-head.

For some applications, the driver is configured to limit an extent to which the winder can wind up the spring.

For some applications, the torsion spring is a torsion bar. For some applications, the torsion spring is a spiral torsion spring. For some applications, the torsion spring is a torsion fiber.

For some applications, the drive-head is sterilized. For some applications, the anchor is sterilized. For some applications, the torsion spring is sterilized. For some applications, the winder is sterilized. For some applications, the driver is sterilized.

For some applications, the winder includes a slip-clutch device. For some applications, the winder includes a pawl and a ratchet gear. For some applications, the winder includes a ratchet gear, and/or the driver includes a pawl that serves as the detent.

For some applications, the release interface is operatively coupled to the pawl such that actuation of the release interface triggers the spring to unwind by disengaging the pawl from the ratchet gear.

For some applications, at a distal portion of the driver, the driver has an eyelet, and/or the detent includes a wire that extends through the eyelet to engage the drive-head.

For some applications, the wire is operatively coupled to the release interface, such that actuation of the release interface triggers the spring to unwind by disengaging the wire from the drive-head.

For some applications, the driver further includes a sleeve, extending from the proximal portion of the driver to the distal portion of the driver, the eyelet being fixed to the sleeve; and/or the driver includes a driveshaft that extends through the sleeve, operatively couples the winder to the drive-head, and/or serves as the torsion spring.

For some applications, the eyelet is disposed inside the sleeve.

In accordance with some applications, a method for anchoring to a tissue of a subject, the method includes transluminally advancing, to the tissue, a distal end of a driveshaft of a driver, the distal end of the drive shaft being engaged with the anchor; and/or subsequently, driving the anchor into the tissue by actuating a release interface at a proximal end of the driver in a manner that releases a torsion spring of the driver to rotate the distal end of the driveshaft.

For some applications, the method further includes winding up the spring. For some applications, the method further includes engaging the driver with the head.

For some applications, the method further includes, subsequently to advancing the tissue-engaging element into the tissue, disengaging the driver from the anchor and withdrawing the driver from the subject.

For some applications, the system/apparatus further includes a detent operatively coupled to the driveshaft and to the release interface, such that activating the release interface includes removing the detent from a position that maintains the tensioned state.

For some applications, the method further includes, responsively to activating the release interface, unscrewing the tissue-engaging element from the tissue by applying reverse torque to the interface using the driver.

In accordance with some applications, a system and/or an apparatus for anchoring an anchor to tissue includes a driver that includes a driveshaft having a distal end that is configured to be transluminally advanced to the tissue while engaged with the anchor.

For some applications, the system/apparatus can further comprise a torsion spring and/or a winder disposed at a proximal portion of the driver, and operatively coupled to the torsion spring such that operation of the winder winds up the spring, and/or a release interface at a proximal end of the driver, operatively coupled to the winder such that actuation of the release interface releases the spring to unwind in a manner that rotates the distal end of the driveshaft.

For some applications, the driver further includes a drive-head at a distal end of the driveshaft, the drive-head configured to engage the anchor.

For some applications, the system/apparatus further includes the anchor, and the drive-head is configured to fit into a cavity in a head of the anchor.

For some applications, the system/apparatus further includes the anchor, and the drive-head is configured to define a cavity that fits over a head of the anchor.

For some applications, the driver further includes a detent, configured to maintain the spring in a tensioned state.

For some applications, the detent is at the proximal portion of the driver.

For some applications, the detent is at a distal portion of the driver.

Any of the above systems, devices, apparatuses, etc. in this summary can be sterilized (e.g., with heat, radiation, ethylene oxide, hydrogen peroxide, etc.), and the methods herein can comprise (or in some additional methods consist of) sterilization of one or more of the systems, devices, apparatuses, etc. herein (e.g., with heat, radiation, ethylene oxide, hydrogen peroxide).

The present invention will be more fully understood from the following detailed description of applications thereof, taken together with the drawings, in which:

### BRIEF DESCRIPTION OF THE DRAWINGS

FIGS. 1A-B show a tissue anchor having dual coaxial helical tissue anchoring elements attached to a single head, in accordance with some applications;

FIG. 2 shows a tissue anchor having dual coaxial tissue-engaging elements in which the inner tissue-engaging element has an opposite handedness to the outer tissue-engaging element, in accordance with some applications;

FIGS. 3 and 4A-G show a tissue anchor with two sequentially advanceable coaxial helical tissue-engaging elements, in accordance with some applications;

FIGS. 5A-B, 6A-D, and 7A-D show two semi-independent helical tissue-engaging elements with inter-fitting heads, in accordance with some applications;

FIGS. 8A-C and 9A-D show a tissue anchor comprising two different types of tissue-engaging elements with inter-fitting heads, in accordance with some applications;

FIGS. 10A-C show a tissue anchor having multiple supplementary spring-loaded hooks attached to the proximal part of the anchor, in accordance with some applications;

FIGS. 11A-D show a tissue anchor having supplementary support provided by multiple short barbs protruding radially from a proximal portion of the anchor, in accordance with some applications;

FIGS. 12A-D show a tissue anchor having supplementary support provided by a ring having circumferentially-directional lobes protruding radially from a proximal portion of the anchor, and fixedly attached to the anchor;

FIGS. 13A-B, and 14A-F show a tissue anchor having a head with an internal hollow connected to a port, and an open tissue-facing surface, in accordance with some applications;

FIGS. 15A-E show a tissue anchor having a helical tissue-engaging element and a head comprising a torque limiter, in accordance with some applications;

FIGS. 16A-E show a tissue anchor having a helical tissue-engaging element and an outer compressible sleeve, in accordance with some applications;

FIGS. 17A-D show a tissue anchor having a threaded tissue-engaging element having an outer compressible sleeve, in accordance with some applications;

FIGS. 18A-B and 19A-B show a tissue anchor having a first helical tissue-engaging element and a second expandable helical tissue-engaging element, in accordance with some applications;

FIGS. 20A-C show an expandable tissue anchor, in accordance with some applications;

FIGS. 21A-C show a tissue anchor having an expandable head, in accordance with some applications;

FIGS. 22A-C and 23A-C show a tissue anchor having a helical tissue-engaging element and supplementary prongs, in accordance with some applications;

FIGS. 24A-B and 25A-C show a tissue anchor having a sharp-tipped, removable inner insert holding a series of flexible prongs, in accordance with some applications;

FIGS. 26A-C and 27A-C show a tissue anchor having inflatable panels, in accordance with some applications;

FIGS. 28A-C and 29A-C show a tissue anchor having a suction-cup mechanism to create a vacuum between the tissue and the anchor, in accordance with some applications;

FIGS. 30A-C show a tissue anchor having a slidable ring for indicating position of the helical tissue-engaging element, in accordance with some applications;

FIGS. 31A-C and 32A-C show a tissue anchor having an indictor wire for indicating a position of a tissue-engaging element within the tissue, in accordance with some applications;

FIGS. 33A-E and 34 show a tissue anchor having multiple shape-memory hooks configured to provide a counterforce to the anchor, in accordance with some applications; and

FIGS. 35A-D and 36A-D show delivery tools and drivers configured to deliver an anchor, in accordance with some applications.

### DETAILED DESCRIPTION

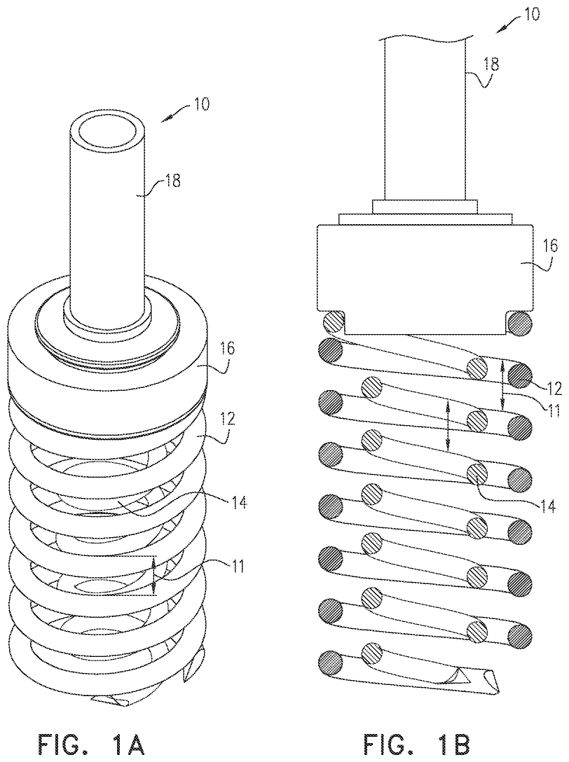

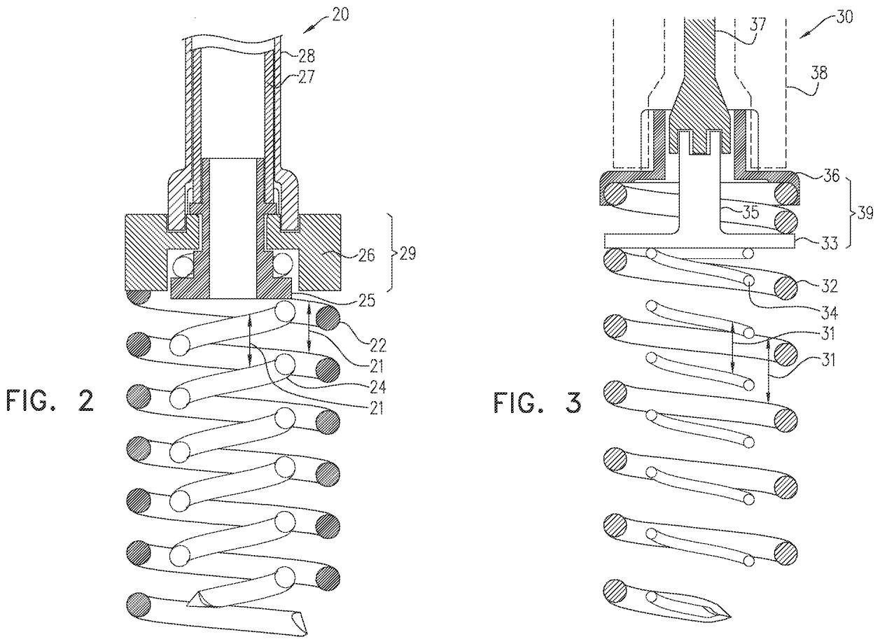

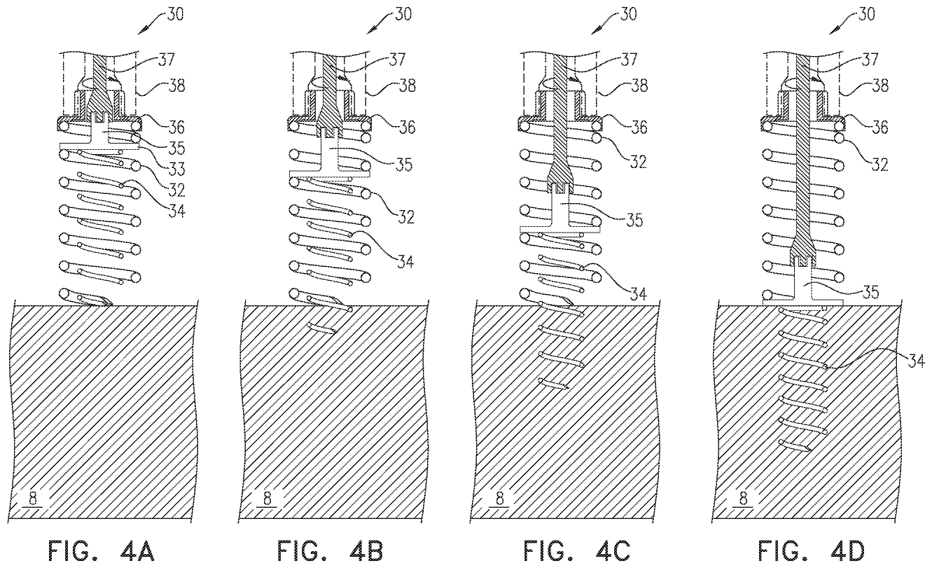

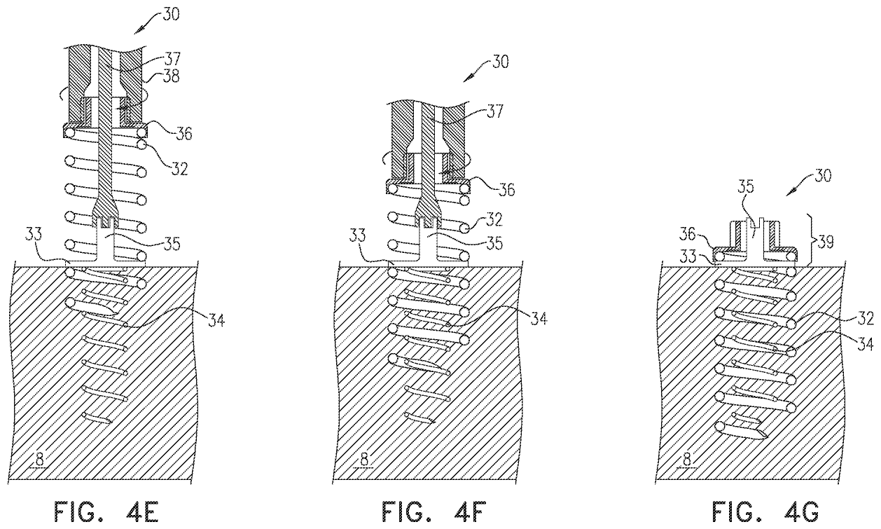

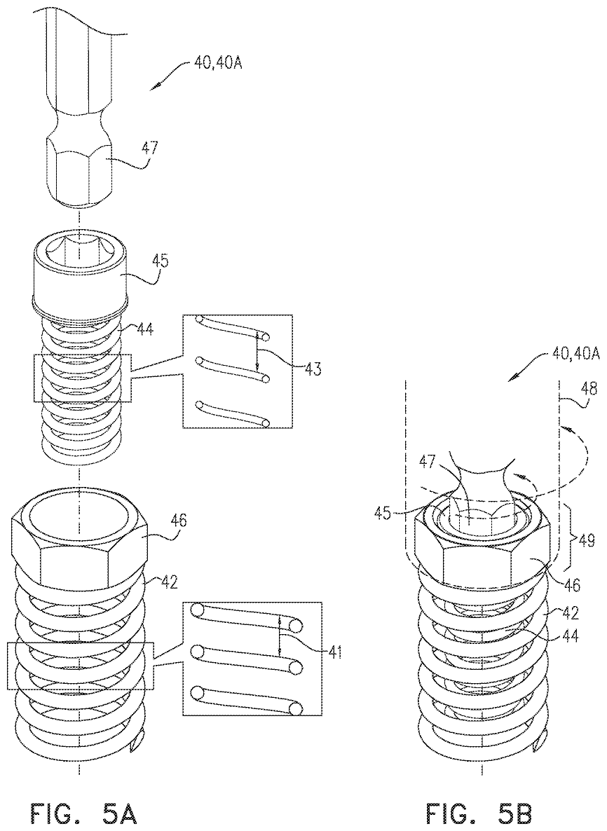

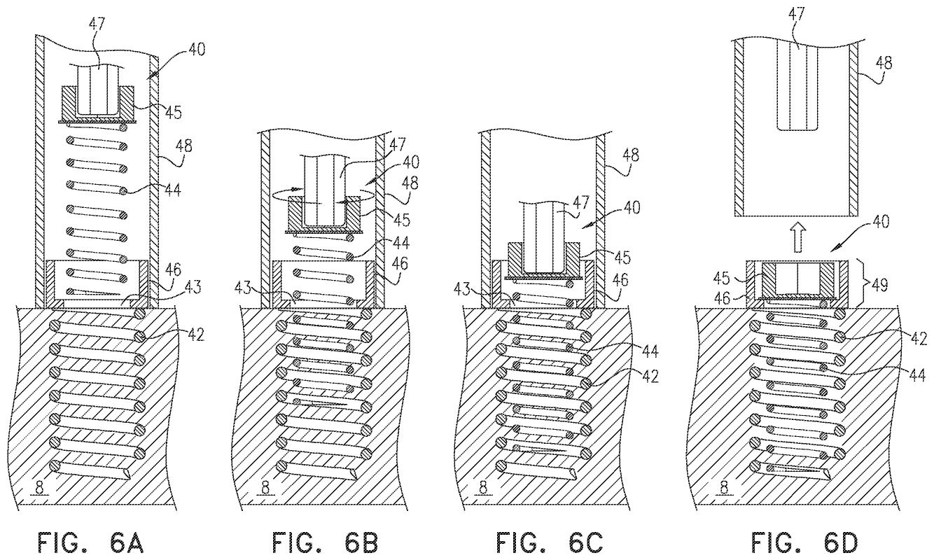

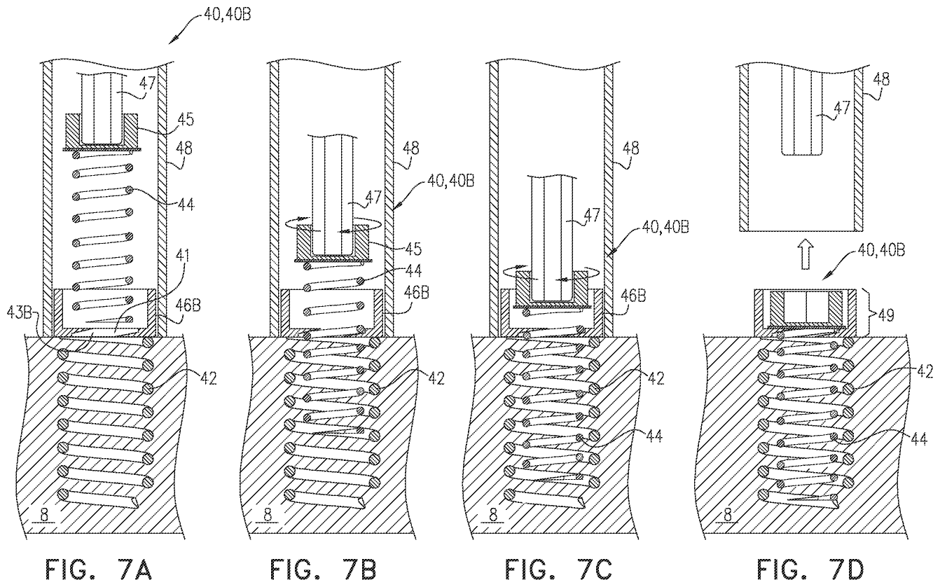

Reference is made to FIGS. 1A-B, 2, 3, 4A-G, 5A-B, 6A-D, and 7A-D, which are schematic illustrations of various systems, each comprising a coaxial helical tissue anchor, in accordance with some applications. FIGS. 1A-B show a tissue anchor 10, FIG. 2 shows a tissue anchor 20, FIG. 3 and FIGS. 4A-G show a tissue anchor 30, and FIGS. 5A-B, 6A-D and 7A-D show a tissue anchor 40A and a tissue anchor 40B. Each of these systems can also comprise a driver for transluminally advancing and anchoring the respective anchor.

The coaxial helical tissue anchor can comprise an outer helical tissue-engaging element and an inner helical tissue-engaging element. These two helical tissue-engaging elements are sized such that the inner diameter of the outer tissue-engaging element is greater than the outer diameter of the inner tissue-engaging element. The two helical tissue-engaging elements are connected at a head assembly. For some applications the head assembly is fixedly coupled to both helical tissue-engaging elements, such that the helical tissue-engaging elements are fixed with respect to each other.

For some applications, the head assembly comprises a first head (e.g., an inner head) fixed to the inner tissue-engaging element, and a second head (e.g., an outer head) fixed to the outer tissue-engaging element.

The first and second heads can be rotationally coupled to each other (e.g., as shown for anchors 20, 30, and 40). For some applications in which the first and second heads are rotationally coupled to each other, the first and second heads are nonetheless axially fixed with respect to each other (e.g., as shown for anchor 20).

For some applications, the first and second heads are rotatable independently of each other (e.g., as shown for anchors 30 and 40). For some applications, the anchor and/or its driver can be configured such that rotation of the inner and outer heads is linked (e.g., as shown for anchor 20).

For some applications, the anchor can be considered to comprise two subunits, the first head and the inner tissue-engaging element collectively defining a first subunit, and the second head and the outer tissue-engaging element collectively defining a second subunit.

For some applications, the inner and outer helical tissue-engaging elements can have the same pitch, i.e., the height of one complete tissue-engaging elements turn is the same for both tissue-engaging elements (e.g., as shown for anchors 10, 20, and 30). For some applications, the tissue-engaging elements can have different pitches and/or handedness (e.g., as shown for anchors 40). It is to be noted that various combinations of handedness and pitch can be used for some applications without limiting to the particular examples shown in FIGS. 1A-7D.

FIGS. 1A-1B show a tissue anchor 10 having dual coaxial helical tissue-engaging elements, e.g., tissue anchoring screws, according to some applications. Anchor 10 comprises a head 16, an outer helical tissue-engaging element 12, and an inner helical tissue-engaging element 14. Both helical tissue-engaging elements 12 and 14 are fixedly attached to head 16.

FIG. 1A shows an isometric view of the tissue anchor 10, in accordance with some applications. FIG. 1B shows tissue anchor 10 in a cutaway side view. Tissue-engaging elements 12 and 14 are aligned coaxially, have the same pitch 11, and have the same handedness.

Tissue anchor 10 can be advanced into a target tissue using a driver 18, that serves, e.g., as the head of a screwdriver, to screw anchor 10 into the tissue. Both tissue-engaging elements rotate in the same direction and concurrently because they are attached to a single head, and their advancement occurs at the same rate because they have the same handedness and pitch.

FIG. 2 shows a tissue anchor 20 having dual coaxial helical tissue-engaging elements 22 and 24. In contrast to anchor 10, tissue-engaging elements 22 and 24 can have opposite handedness, i.e., they can be screwed into tissue by rotation in opposite directions to each other.

Anchor 20 comprises a head assembly 29 that comprises a first head 25 (e.g., an inner head) and a second head 26 (e.g., an outer head). Outer tissue-engaging element 22 is attached to second head 26, and inner tissue-engaging element 24 is attached to first head 25. First head 25 and second head 26 are rotatably coupled to each other and axially fixed with respect to each other. For example, and as shown, second head 26 can comprise a cylindrically shaped ring, and first head 25 can comprise a core that is secured within second head 26 such that the first head is rotatable but not axially movable with respect to the second head.

The head assembly 29 is rotated by a driver, which concurrently advances both tissue-engaging elements 24, 22 into the tissue. The driver has two components, a first component 27 and a second component 28. Driver component 27 engages and is configured to rotate head 25, and driver component 28 engages and is configured to rotate head 26. Driver components 27 and 28 can be removably engageable with heads 25 and 26, respectively, such that, after the tissue anchor 20 has been anchored, the driver is detachable from head assembly 29.

Heads 25 and 26 cooperate with each other to facilitate anchoring, i.e., when head 26 is rotated by driver component 28, advancing tissue-engaging element 22, head 25 is simultaneously rotated driver component 27 in the opposite direction, advancing tissue-engaging element 24 into the tissue. Thus, the tissue-engaging elements enter the tissue concurrently, but screw in opposite directions as they enter the tissue. The head assembly 29 can also include a wire locking feature which can be a pulley mechanism mounted on the concentric heads 25, 26 (not shown).

For some applications, in order that tissue-engaging elements 22 and 24 advance into the tissue at the same axial rate, they have the same pitch 21 (e.g., as shown), and are rotated at the same rotational rate as each other during anchoring. For other applications, the pitch of tissue-engaging element 22 differs from the pitch of tissue-engaging element 24, and the tissue-engaging elements are rotated at different rotational rates to each other. The rotational rate ratio (i.e., the ratio between the rotational rate of element 22 and element 24) is known and fixed, such that, e.g., if the pitch of tissue-engaging element 24 were half that of tissue-engaging element 22, the rotational rate of element 24 (and thus of head 25) would be double that of element 22 (and thus of head 26). The driver components can be calibrated such that each component is suited to a particular pitch ratio. Thus, driver components 27, 28 are linked in a manner such that the rates of rotation of heads 25, 26 need not be identical, but fixed relative to each other such that tissue-engaging elements 22, 24 enter the tissue essentially concurrently.

The application shown in FIG. 2, i.e., a tissue anchor 20 with two tissue-engaging elements having opposite handedness, hypothetically and advantageously promotes enhanced locking into the tissue compared to a tissue anchor having a single tissue-engaging element. For some applications, this same advantage can also apply compared to a tissue anchor having dual helical tissue-engaging elements that have the same handedness.

FIGS. 3 and 4A-G show a tissue anchor 30 with two sequentially-advanceable coaxial helical tissue-engaging elements 32 and 34, e.g., helical screws, according to some applications. The coaxial tissue-engaging elements 32 and 34 have the same handedness and the same pitch 31.

Tissue anchor 30 further comprises a head assembly 39 that comprises a first head 35 (e.g., an inner head) and a second head 36 (e.g., an outer head). First head 35 can comprise or defines a runner 33 and is fixed to inner tissue-engaging element 34. Second head 36 is fixed to the outer tissue-engaging element 32. Runner 33 is configured to advance around the turns of outer tissue-engaging element 32 as inner tissue-engaging element 34 is advanced into the tissue, e.g., the driving of element 34 into the tissue in response to rotation of head 35 is at least partly guided by runner 33 running around the turns of element 32. Runner 33 can be, e.g., t-shaped, a partially helicoid disc (or a helicoid partial disc), fingers, or another convenient shape that allows advancement along the outer tissue-engaging element. The two heads are independently advanceable, and optionally lockable once both tissue-engaging elements have been inserted into the tissue.

A driver with two components 37, 38, e.g., two reversibly couplable coaxial drive shafts, can be used to advance the anchors into the tissue. Using driver component 37, inner tissue-engaging element 34 can be advanced first into the target tissue, with runner 33 running around the turns of element 32 (FIGS. 4A-D). As head 35 is turned by rotating driver 37, inner tissue-engaging element 34 advances with respect to outer tissue-engaging element 32. That is, outer tissue-engaging element 32 serves as a rail for runner 33, facilitating advancement of tissue-engaging element 34 into the tissue. Head 35 (e.g., runner 33 thereof) typically comes to rest on a surface of the tissue 8 once inner tissue-engaging element 34 has been fully anchored into the tissue. Subsequently, driver component 38 rotates head 36, and outer tissue-engaging element 32 is thus screwed over runner 33 and into the tissue (FIGS. 4E-G). Inversely to during the advancement of inner tissue-engaging element 34, the engagement between runner 33 and outer tissue-engaging element 32 now facilitates advancement of the outer tissue-engaging element. When outer tissue-engaging element 32 has been fully screwed into the tissue, head 36 typically rests on head 35 (e.g., runner 33 thereof), sandwiching head 35 (e.g., the runner) between head 36 and a surface of target tissue 8. Runner 33 can facilitate locking of the anchors to each other (e.g., locking of heads 35 and 36 to each other), which can advantageously provide force distribution between both tissue-engaging elements. It is hypothesized that, compared to advancing two elements simultaneously, advancing two tissue-engaging elements sequentially can require less torque and/or can be less traumatic to tissue.

FIGS. 5A-B, 6A-D, and 7A-D are schematic illustrations of a tissue anchor 40 with two semi-independent helical tissue-engaging elements with inter-fitting heads. Together, heads 45 and 46 comprise a head assembly 49. Inner tissue-engaging element 44 is fixed to first head 45. Outer tissue-engaging element 42 is fixed to second head 46.

A driver having two components 47, 48 is used to advance tissue anchor 40 into the tissue. Driver component 48 reversibly attaches to, e.g., fits around, head 46, and driver component 47 reversibly attaches to, e.g., fits within, head 45.

Tissue-engaging elements 42 and 44 can have the same or different pitch. FIGS. 5A-B and 6A-D show a tissue anchor 40A, which is a variant of tissue anchor 40, whose inner and outer tissue-engaging elements have the same handedness. FIGS. 7A-D show a tissue anchor 40B, which is a variant of tissue anchor 40, whose inner and outer tissue-engaging elements have opposite handedness to each other.

The geometry of first head 45 and second head 46 can allow independent driving and rotation of the anchors relative to each other. The independent rotation and driving can be accomplished, e.g., by having head 46 with a noncircular (e.g., hexagonal) outer shape for reversible coupling to driver component 48, and an inner cylindrical shape. By contrast, head 45 can have a circular outer shape to fit rotatably within head 46. Head 45 can have an inner non-cylindrical, e.g., hexagonal shape to allow rotational locking to driver component 47.

FIGS. 5A-B show components of tissue anchor 40A in both pre- and post-anchoring configurations. In FIG. 5A, inner tissue-engaging element 44 and head 45 are shown separately from outer tissue-engaging element 42 and head 46. Driver component 47 has a non-circular (e.g., hexagonal) shape, and reversibly engages head 45 by fitting into an opening with a complementary shape in head 45. Not shown in FIG. 5A is a driver component 48, which reversibly engages to head 46. In FIG. 5B, the same components are shown in a fully-assembled configuration, such that tissue-engaging element 44 is disposed within tissue-engaging element 42, and head 45 is fitted within head 46, head 45 and head 46 collectively defining a head assembly 49. FIG. 5B could also represent a later step in an anchoring process, e.g., showing a point at which the anchor 40A has been fully advanced into the tissue by a driver having components 47 and 48.

Outer head 46 can have an opening 43 in its central region through which tissue-engaging element 44 can be advanced. Opening 43 can have a diameter greater than the outer diameter of tissue-engaging element 44, but smaller than the diameter of inner head 45, such that tissue-engaging element 44 can advance therethrough, but is stopped when head 45 rests on the outer edge of opening 46A. In such applications, and as shown, e.g., in FIGS. 6A-D, outer tissue-engaging element 42 is anchored into the tissue first, and inner tissue-engaging element 44 is subsequently advanced through opening 43 and anchored into the tissue.

Using driver component 48 to rotate head 46, outer tissue-engaging element 42 can be advanced first into the target tissue. As head 46 is turned by rotating driver 48, outer tissue-engaging element 42 advances with respect to inner tissue-engaging element 44. Head 46 typically becomes pressed against the surface of the tissue 8 8 once outer tissue-engaging element 42 has been fully anchored into the tissue (FIG. 6A).

Subsequently, driver component 47 rotates head 45, and inner tissue-engaging element 44 is thus screwed into the tissue (FIGS. 6B-C). When inner tissue-engaging element 44 has been fully screwed i.e., when anchor 40 is fully anchored, into the tissue, head 45 typically rests against and/or within head 46, such that head 46 is sandwiched between head 45 and surface of the tissue 8. Inter-fitting of heads 45 and 46 to each other can advantageously provide a more even distribution of force between both tissue-engaging elements than would be possible if the heads were not inter-fitted.

FIGS. 7A-D illustrate similar steps to those shown in FIGS. 6A-D, but for anchor 40B. As well as having opposite-handedness (as described hereinabove), anchor 40B has an outer head 46B that defines an opening 43B that defines helical threads 41 that match the turns of inner helical tissue-engaging element 44. Threads 41 are configured to match both the pitch and handedness of inner tissue-engaging element 44 such that the turns of tissue-engaging element 44 advance along the inner helical thread as tissue-engaging element 44 is screwed into the tissue. For some applications, threads 41 are configured such that tissue-engaging element 44 can only be advanced through opening 43B by rotation. Although FIGS. 6A-D show both helical tissue-engaging elements having the same handedness, and FIGS. 7A-D show the inner tissue-engaging element having an opposite handedness to the outer tissue-engaging element, these are not limiting examples. For example, anchor 40 of FIGS. 6A-D can have opposite handedness, and anchor 40B of FIGS. 7A-D can have same-handedness. Similarly, threads 41 are neither essential nor limited to the exemplary application illustrated in FIGS. 7A-D; they can be included in other applications, such as that shown in FIGS. 6A-D.

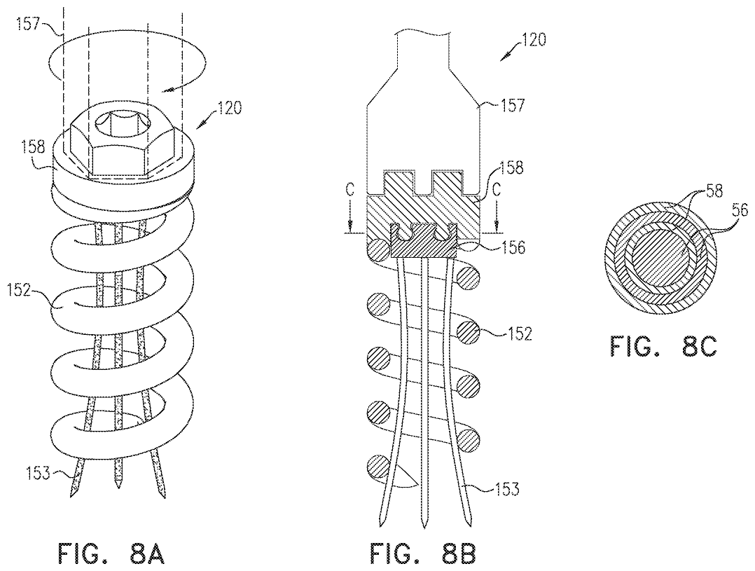

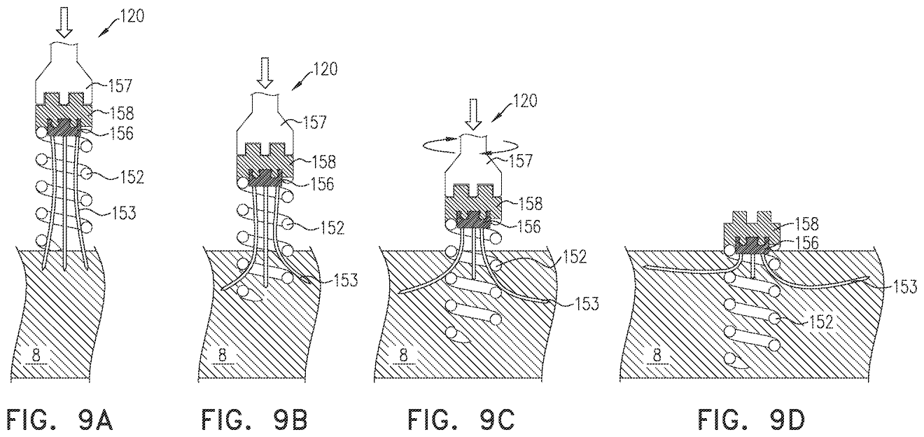

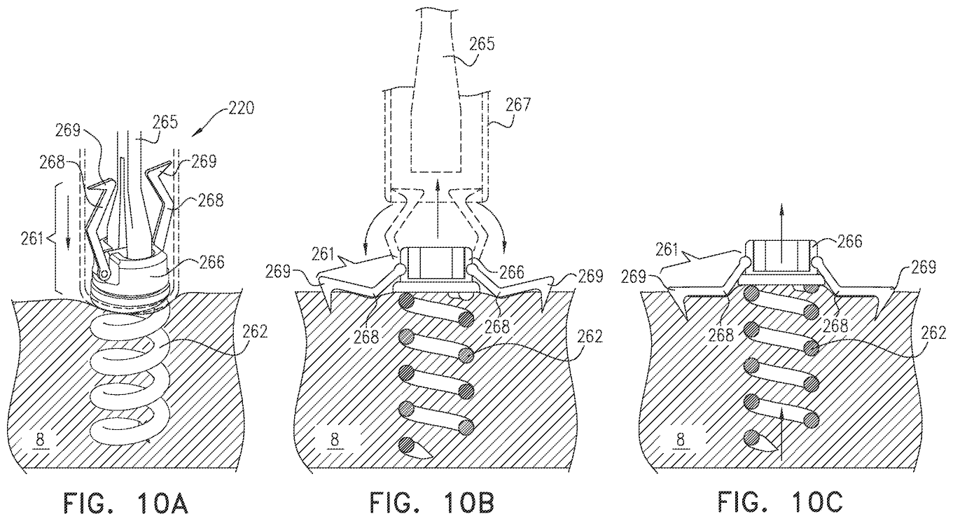

Reference is made to FIGS. 8A-C and 9A-D, which are schematic illustrations of a tissue anchor 120, in accordance with some applications. FIG. 8A shows a perspective view of anchor 120, FIGS. 8B-C are cross-sections of the anchor, and FIGS. 9A-D show steps in anchoring the anchor.

Tissue anchor 120 comprises a first head 158 fixed to a helical tissue-engaging element 152, e.g., a helical tissue-penetrating screw. Tissue-engaging element 152 extends away from first head 158, helically around an elongate space, and terminates at a sharpened distal tip. The elongate space around which tissue-engaging element 152 extends is substantially cylindrical and disposed along a longitudinal axis of the anchor.

A second head 156 is coupled to first head 158 in a manner in which second head 156 is rotatable, but axially fixed, with respect to first head 158, e.g., by a rotatable snap-fit mechanism. The first head can further comprise a non-circular protrusion configured to reversibly couple to a driver.

Fixed to the second head are multiple prongs 153 that extend from second head 156 distally through the elongate space. Prongs 153 can be fixed to second head 156 in a ring-like arrangement. As described in more detail hereinbelow, prongs 153 are arranged with respect to helical tissue-engaging element 152 such that rotation of first head 158 with respect to second head 156 feeds the prongs laterally outward between progressively proximal turns of the tissue-engaging elements.

Prongs 153 (e.g., tines, barbs, or spikes) are flexible, such that they can be deflected and/or deformed by tissue-engaging element 152, as described hereinbelow. For some applications, prongs 153 comprise a plastically-deformable material such as stainless steel or a plastic polymer. For some applications, prongs 153 comprise an elastically-deformable material such as nitinol, or an elastic polymer. Prongs 153 can number two, three, four, or any number of prongs deemed to provide the desired level of tissue support.

For some applications, and as shown, prongs 153 can be long enough such that, when the prongs extend through the elongate space defined by tissue-engaging element 152, they extend distally out of the elongate space, i.e., beyond the distal-most turn of the tissue-engaging element. This can be considered an initial (e.g., pre-use) state of anchor 120.

For some applications, in the initial state, the tips of prongs 153 do not extend laterally beyond helical tissue-engaging element 152. For some such applications, the prongs can be configured to expand laterally upon being pressed axially into tissue, so that their tips then do extend laterally beyond tissue-engaging element 152.

As shown in FIG. 8B, the outer diameter of head 156 can be sized to be less than the inner diameter of helical tissue-engaging element 152, i.e., such that head 156 can fit within tissue-engaging element 152.

In an exemplary application, e.g., as shown in FIGS. 8B-C and 9A-D, the rotatable coupling between head elements 156 and 158 can be provided by one or more riders defined by one of the head elements, riding in/on a track defined by the other of the head elements. For example, protrusions of one of the head elements can protrude into a circular depression in the other of the head elements. For example, and as shown, head 158 can have has a circular, i.e., annular, protrusion on its under surface, i.e., the surface facing the tissue when the system/apparatus is positioned for insertion into a target tissue. The circular protrusion on the underside of head 158 is sized such that it fits rotatably into a corresponding circular depression on the upper side of head 156. For some applications, the location of the protrusion and depression are reversed, i.e., the depression is found on head 158 and the protrusion on head 156.

For some applications, the anchor defines a track and a rider, and the second head is coupled to the first head via the rider being slidably coupled to the track. The track can be annular, and the rider can be a set of riders being distributed in a ring formation and slidably coupled to the track by protruding into the track.

Head 158 can be rotated by means of a driver 157, which can engage head 158 by fitting a complementary shape of head 158, e.g., as shown. As tissue-engaging element 152 is screwed into the target tissue, prongs 153 advance into the tissue without rotation, e.g., due to head 158 rotating with respect to head 156 as head 158 pushes head 156 toward the tissue. This design allows tissue-engaging element 152 to be rotated without concurrently rotating prongs 153, which could result in undesirable tissue damage. Both tissue-engaging element 152 and prongs 153 are concurrently advanced into the tissue. Prongs 153 mechanically cooperate with helical tissue-engaging element 152, as described in the following steps.

In a typical application, the following procedure can be followed to insert the tissue anchor 120, as illustrated in FIGS. 9A-D. The anchor 120 comprises head 158 attached to helical tissue-engaging element 152, and head 156 attached to prongs 153. Driver 157, reversibly engaged with head 158, advances anchor 120 toward tissue 8 (FIG. 9A). For some applications, and as shown, tissue anchor 120 is positioned such that the tips of prongs 153 contact and/or penetrate into tissue 8. As driver 157 is rotated, tissue anchor 120 begins to move into the target tissue (FIG. 9B). As tissue-engaging element 152 is screwed into the target tissue, a tip of tissue-engaging element 152 passes between two of prongs 153 (FIG. 9B). As tissue-engaging element 152 continues to be progressively screwed into the target tissue, prongs 153 are progressively fed (e.g., pushed) laterally/radially outward between progressively proximal turns of tissue-engaging element 152. The laterally/radially outward movement of prongs 153 can be facilitated by axial pressure from driver 157 pressing the prongs into the target tissue.

The order and specific path of prongs 153 advancing laterally into the tissue can vary from the exemplary application shown in FIGS. 9A-D.

A potential advantage of the application shown in FIGS. 8A-C and 9A-D include multiple anchoring elements having different structural features and mechanical properties. Another potential advantage is that tissue-engaging element 152 supports the prongs 153 after implantation more strongly than the prongs would hold themselves in a tissue anchor having only prongs and no tissue-engaging elements.

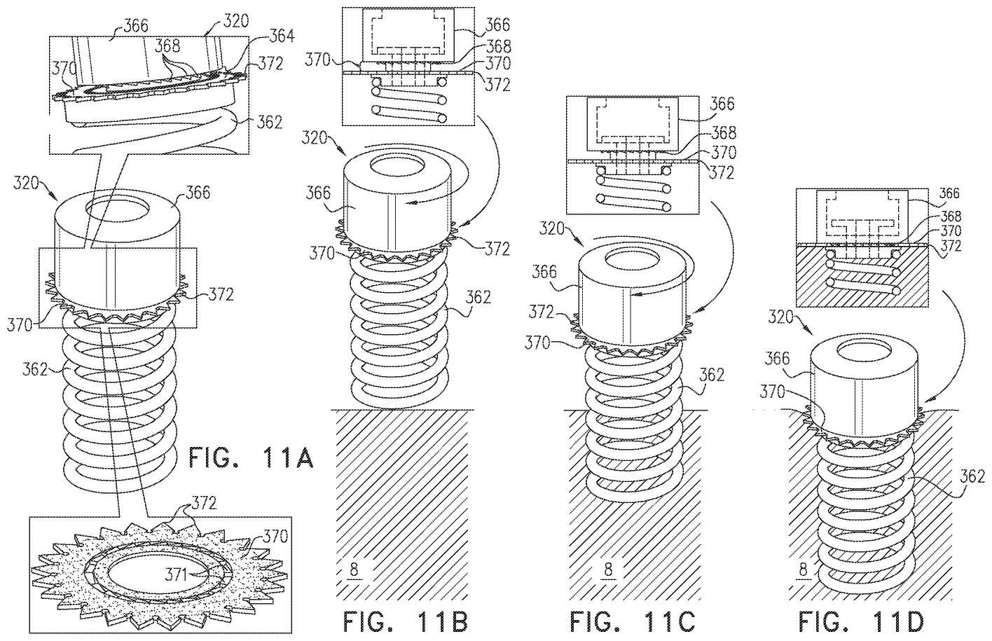

Reference is made to FIGS. 10A-C, 11A-D, and 12A-D, which are schematic illustrations of tissue anchors that have a primary tissue-engaging element and one or more supplementary tissue-engaging elements, in accordance with some applications.

FIGS. 10A-C show a tissue anchor 220 that has a primary tissue-engaging element 262 and one or more, e.g., a set of, supplementary tissue-engaging elements 261, in accordance with some applications. For example, anchor 220 can comprise 2, 3, 4, or any number of supplementary tissue-engaging elements deemed relevant for supplementary tissue anchor stabilization.

Tissue-engaging element 262 extends distally from a head 266 of anchor 220. Tissue-engaging element 262 is shown as a helical tissue-engaging element (e.g., a helical tissue screw), but the scope of the disclosure includes tissue-engaging element 262 being another type of tissue anchoring element, such as (but not limited to) a dart or staple. Primary tissue-engaging element 262 defines a longitudinal axis of the anchor—in the case of it being a helical tissue-engaging element, by extending helically around the axis.

Supplementary tissue-engaging elements 261 are coupled to head 266. Each supplementary tissue-engaging element 261 can comprise a spring-loaded hook 269, e.g., a grip, a tooth, or a barb. Each hook 269 can have a point angled toward the target tissue. Hooks 269 can be attached to anchor head 266 by arms 268 having, e.g., elastic properties, a hinge, or a double-jointed elbow-type articulation.

As shown in FIG. 10A, during primary anchoring of the tissue anchor 220, e.g., screwing of the primary tissue-engaging element into the target tissue, the hooks 269 and arms 268 of supplementary tissue-engaging element 261 are constrained in an “up” position, in which they can extend above (i.e., proximally from) the head (i.e., farther than the anchor head from the tissue). Constraining hooks 269 can be accomplished by the anchor being disposed within a delivery catheter 67 or driver sleeve. Subsequently, e.g., once the anchor head is disposed against the tissue surface, the hooks and arms are released, as shown in FIG. 10B, such that they deflect distally (i.e., “downward” toward the primary tissue-engaging element and the tissue) to hook into the surface of the tissue 8, providing supplementary anchoring.

For some applications, the downward movement of the hooks includes lateral or radial outward swing of the arms 268 in an arc. The final movement of the arc can include a slight medial or inward diversion of the hooks. The arms 268 can be deflectable further distally, i.e., toward the primary tissue-engaging element, after anchoring. The further deflection of the arms, i.e., via the elastic properties, hinge, or elbow-type articulation, enables the hooks to remain anchored within the tissue 8 even if the anchor head is pulled proximally, as shown in FIG. 10C, e.g., due to forces applied to and/or by an implant that is anchored by anchor 220. For example, testing of full anchoring can be performed by pulling proximally on head 266, e.g., by driver 265, 267, such that supplementary tissue-engaging elements 261 articulate with respect to head 266. The action of applying a proximal pulling force to the head can further enable at least some of the pulling force to be distributed to the tissue via the supplementary tissue-engaging elements.

The supplementary anchoring provided by supplementary tissue-engaging elements 261 is hypothesized to produce only minimal tissue trauma, while providing additional stabilization. Supplementary tissue-engaging elements 261 can distribute forces over a wider area of surface of the tissue 8 than would be achieved by a similar anchor that has only primary tissue-engaging element 262. The additional stabilization can advantageously prevent the tissue anchor from either pulling out completely, or from collapsing to one side within the target tissue.

FIGS. 11A-D show a tissue anchor 320 having primary tissue-engaging element 362 attached to a head 366. Anchor 320 further comprises a supplementary tissue-engaging element 370. Anchor 320 has a longitudinal axis extending from the head through the primary tissue-engaging element. Supplementary tissue-engaging element 370 comprises multiple short barbs 372, e.g., serrations, teeth notches, hooks, grips, or other tissue-engaging protrusions that protruding laterally beyond head 366. Barbs 372 can protrude from a proximal portion of the anchor, e.g., at, or just distal to, the anchor head 366. Supplementary tissue-engaging element 370 can be located close to primary tissue-engaging element 362—e.g., between head 366 and the primary tissue-engaging element 362, as shown. As shown, barbs 372 can be arranged circumferentially around the longitudinal axis of the anchor.

For some applications, and as shown, supplementary tissue-engaging element 370 can be shaped as a ring that defines barbs 372, or to which the barbs are coupled. Thus, the outer edge of ring 370 can have a diameter slightly greater than that of head 366, and/or barbs 372 can protrude radially outward beyond the head.

For some applications, tissue-engaging element 370 comprises at least 6, e.g., at least 9, e.g., at least 18, e.g., at least 24, barbs 372.

For some applications, (e.g., for some applications in which primary tissue-engaging element 362 is a screw-in helical tissue-engaging element), supplementary tissue-engaging element 370 can be rotatable with respect to head 366 and/or primary tissue-engaging element 362. For some applications, this rotatability is provided by anchor 320 (e.g., head 366 thereof) defining a circumferential recess 364 within which ring-shaped supplementary tissue-engaging element 370 is disposed and is freely rotatable. It is hypothesized that this can allow supplementary tissue-engaging element 370 to become stationary upon contacting the tissue, thereby preventing the supplementary tissue-engaging element (e.g., barbs 372 thereof) from tearing the tissue surface when primary helical tissue engaging element 362 continues to be screwed into tissue 8.

For some applications, a clutch comprising complimentary clutch surfaces 368 (e.g., comprising one or more pawls) and 371 (e.g., comprising ratchet teeth) can be used to constrain the rotation of tissue-engaging element 370 in one direction. Ratchet teeth 371 can be added, to the tissue engaging element 370 medially from barbs 372. Once supplementary tissue-engaging element 370 contacts the tissue, it becomes pushed toward clutch surface 368 of head 366, such that clutch surfaces 368 and 371 become engaged. The engagement between clutch surfaces 368 and 371, in combination with gripping of the tissue by barbs 372, is hypothesized to inhibit rotation of tissue-engaging element 362 in a reverse direction, thereby reducing a likelihood of anchor 320 unscrewing.

In FIGS. 11B-D, the tissue anchor is shown being inserted into target tissue 8. When the primary tissue-engaging element 362 is fully anchored, as shown in FIG. 11D, head 366 pushes against the surface of tissue 8 (e.g., creating a valley), and radial barbs 372 become pushed against the surface of the tissue (e.g., into the walls of the valley). The barbs can be dimensioned (e.g., are sufficiently short) to engage the endothelium of the target tissue, but to not penetrate deeply into the tissue.

For applications in which supplementary tissue-engaging element 370 is rotatable with respect to head 366 and/or primary tissue-engaging element, even after supplementary tissue-engaging element engages the tissue, primary tissue-engaging element 362 can continue to be screwed into the tissue while the supplementary tissue-engaging element remain rotationally stationary with respect to the tissue, e.g., so that barbs 372 don't saw into the tissue.

Supplementary tissue-engaging element 370 can comprise a radiopaque substance, such that it enables visualization after implantation by fluoroscopy. For some applications, supplementary tissue-engaging element 370 comprises a shape-memory metal that can be folded within driver 267 such that barbs 372 point proximally and can unfold to their radial position as shown in FIGS. 11B-D upon deployment. In some such applications, driver 267 can hold barbs 372 in a proximal direction in a crimped configuration within the drive shaft, and release them to an open, radial configuration as the driver withdraws after complete anchoring of primary tissue-engaging element 362. In such an exemplary application, the folded edges of ring 370 can have a diameter not substantially greater than the diameter of head 366.

This solution advantageously provides minimally-traumatic supplementary anchoring toward the proximal end of the anchor, and potentially over a wider area of tissue, than a tissue anchor having only a primary tissue-engaging element.

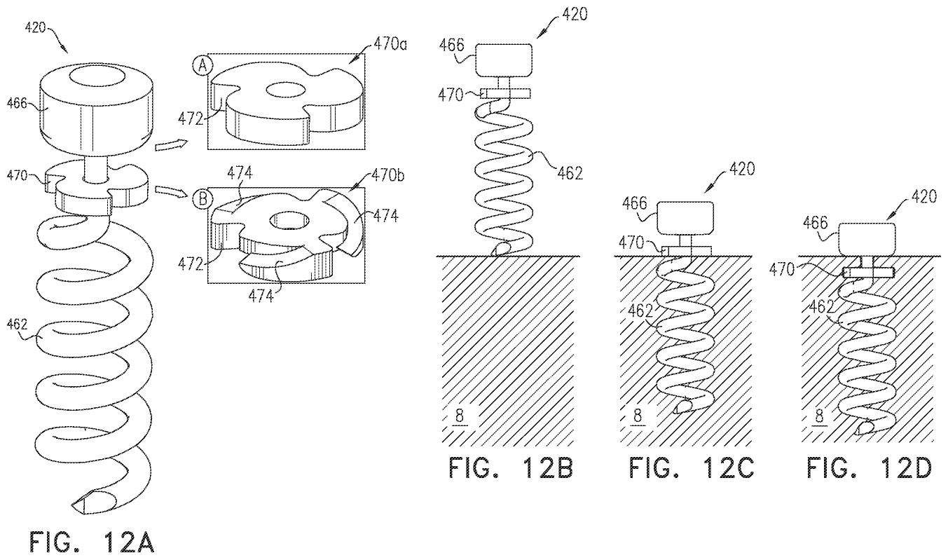

FIGS. 12A-D show a tissue anchor 420 having primary tissue-engaging element 462 attached to a head 466. Similarly to anchor 320, anchor 420 further comprises a supplementary tissue-engaging element 470, e.g., a retention ring, flange, or collar, disposed between head 466 and primary tissue-engaging element 466. Anchor 420 has a longitudinal axis extending from the head through the primary tissue-engaging element. Similarly to retention ring 370, retention ring 470 also comprises multiple protrusions. However, the protrusions of ring 470 have a uniform circumferential directionality, e.g., they all “point” in the same circumferential direction around the longitudinal axis of the anchor. In the example shown, the protrusions of ring 470 are lobes 472 (e.g., resembling blades of a fan or propellor, e.g., as shown), but they could be teeth, serrations, notches, or other tissue-engaging protrusions. Retention ring 470 can be located close to primary tissue-engaging element 462—e.g., between head 466 and the primary tissue-engaging element 462, as shown. Retention ring 470 can be fixedly attached to both head 466 and tissue-engaging element 462, optionally with a small longitudinal gap between head 466 and ring 470.

For some applications, and as shown, lobes 472 are arranged circumferentially around the longitudinal axis of the anchor, having a circumferential directionality that would facilitate anchoring of anchor 420 into tissue 8, but would resist undesired de-anchoring of the anchor. That is, when in contact with tissue, lobes 472 resist rotation of the anchor in one direction (the direction of unscrewing) more than in the opposite direction (the direction of screwing in). Thus, the orientation of lobes 472 for any given anchor may depend on the handedness of primary tissue-engaging element 462. For some applications, tissue-engaging element 470 comprises at least 2, at least 3, e.g., at least 4, e.g., at least 6, e.g., at least 12, lobes 472.

In FIGS. 12B-D, the tissue anchor is shown being driven into target tissue 8. When the primary tissue-engaging element 462 is fully anchored, as shown in FIG. 12D, head 466 pushes against the surface of tissue 8, and lobes 472 of retention ring 470 are disposed within the tissue. If a gap exists between head 466 and retention ring 470, it may become filled with tissue. For some applications, a proximal circumferential surface of each lobe is beveled 474, e.g., as shown as a variant 470b in the enlarged inset B portion of FIG. 12A. For some applications, a distal circumferential surface of each lobe is beveled. Beveling 474 on each lobe 472 may enhance the interaction between ring 470 and the tissue, e.g., facilitating driving of lobes 472 into the tissue (e.g., allowing them to cut into the tissue) or sliding of the lobes across the surface of the tissue, while maintaining their resistance to de-anchoring. However, for some applications lobes 472 are not beveled, e.g., as shown as a variant 470a in the enlarged inset A portion of FIG. 12A.

Supplementary tissue-engaging element 470 can comprise a radiopaque substance, such that it enables visualization after implantation by fluoroscopy. For some applications, this may facilitate decision-making prior to commitment of anchoring of the anchor. For example, primary tissue-engaging element 462 can be driven into tissue 8 until supplementary tissue-engaging element 470 is close to the surface of the tissue (FIG. 12C). At this point anchoring may be assessed (e.g., by imaging and/or by pulling the anchor proximally). Only if the anchoring is determined to be acceptable, the anchor is driven further in order to place supplementary tissue-engaging element 470 into contact with and/or within the tissue (FIG. 12D).

The solution shown in FIGS. 12A-D advantageously provides supplementary anchoring toward the proximal end of the anchor, and potentially maintains torsion and tension of the anchor within the tissue, more efficiently than a tissue anchor having only a primary tissue-engaging element.

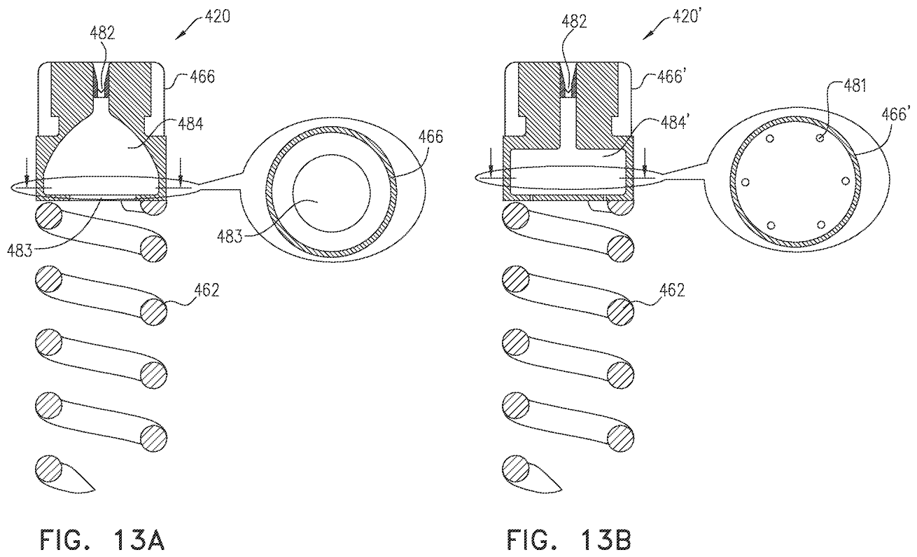

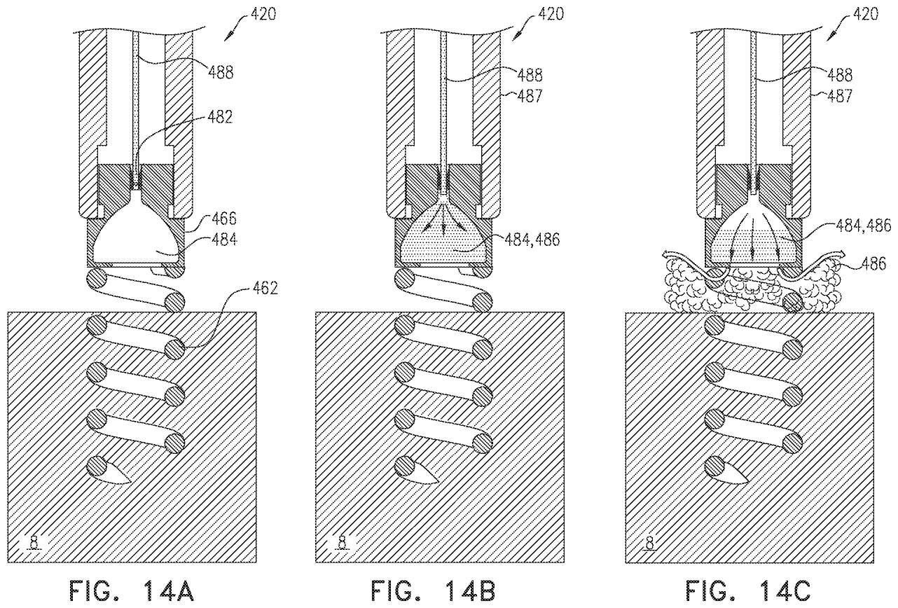

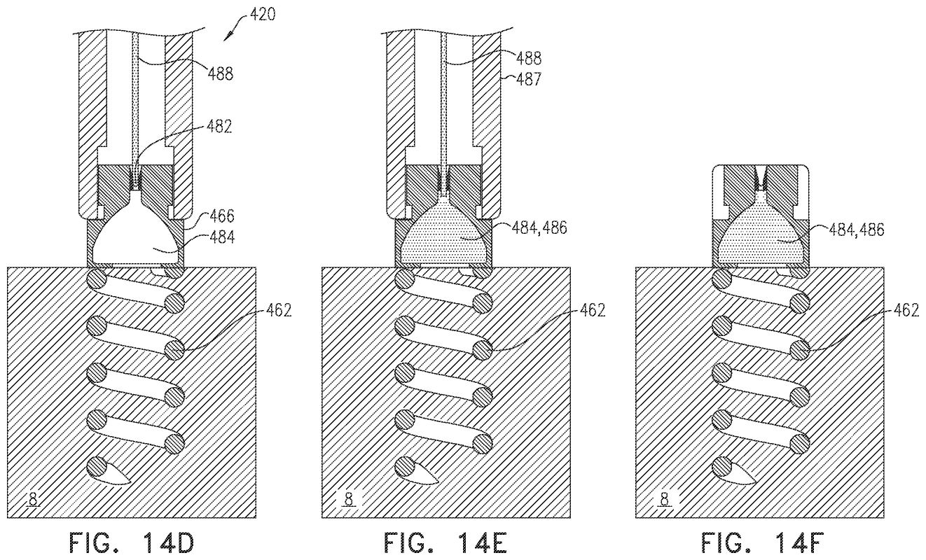

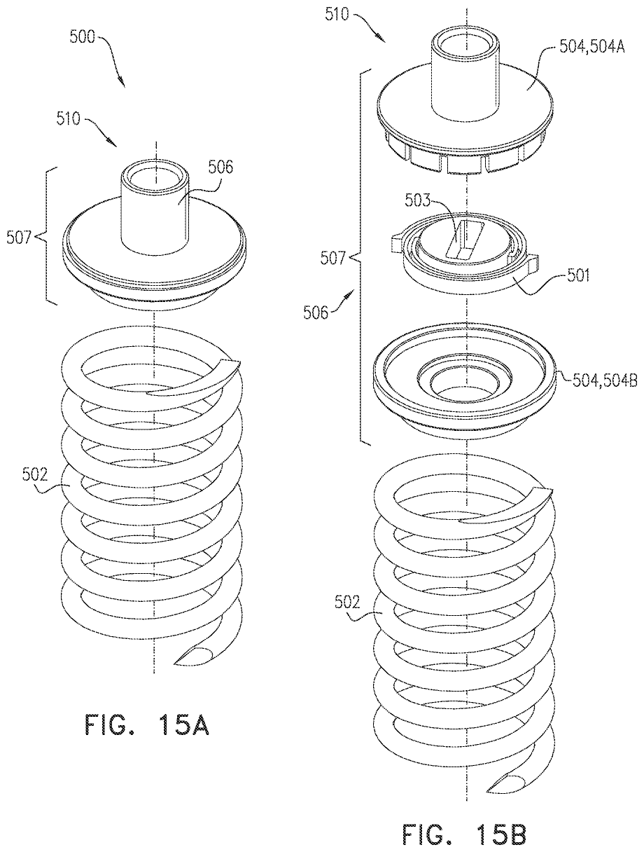

Reference is made to FIGS. 13A-B and 14A-F, which are schematic illustrations of a tissue anchor 420 and a variant thereof, in accordance with some applications. For some applications, e.g., percutaneous cardiac procedures, direct visualization of the tissue anchor during and after implantation is not possible. Thus, it may be desirable to ascertain, using other means, positioning of the anchor, e.g., whether the anchor has been optimally/fully anchored in the tissue.

FIG. 13A shows tissue anchor 420, which comprises a tissue-engaging element 462 fixed to head 466. FIG. 13B shows a tissue anchor 420′, which is a variant of tissue anchor 420, and which comprises a head 466′ in place of head 466. The left portion of each of FIGS. 13A-B represents a longitudinal cross-section through the anchor, and the right portion of each figure represents a horizontal cross-section through the head, as viewed from above.

A port 482 on a surface of the head, e.g., on the upper surface, connects to an internal hollow 484, 484′ defined within the head. The hollow can be in fluid communication with a tissue-facing surface of the head, e.g., through a plurality of small openings 481 (head 466′ of anchor 420′), or through a single larger, central opening 483 (head 466 of anchor 420). The internal hollow can be of any convenient shape. The hollow is dimensioned to contain a predetermined quantity of a liquid, e.g., a contrast agent used for fluoroscopic or radiographic studies.

The contrast agent can be contained within a dispenser comprising a capsule containing the contrast agent (not shown). The capsule can be disposed, e.g., at a distal portion of the driver or at an extracorporeal proximal portion of the driver. For some applications, the capsule can be frangible, and the dispenser is configured to dispense the contrast agent by breaking or piercing the capsule. Alternatively, or additionally, the capsule can be compressible, and the dispenser configured to dispense the contrast agent by compressing the capsule. For some applications, the dispenser comprises a pump.

FIGS. 14A-F show a sequence of steps representing a process of ascertaining an optimal/complete anchoring of tissue anchor 420. A driver 487 comprising an inner channel 488 can be used to insert tissue anchor 420 into the target tissue. Channel 488 can fluidly connect via port 482 to internal hollow 484, e.g., can be fluidly connected to the port upon mechanical engagement of the anchor by the driver. During anchoring of tissue anchor 420 to the target tissue, contrast agent 486 can be dispensed into hollow 484 via channel 488, in order to ascertain optimal/complete anchoring. The contrast agent can be dispensed into the internal hollow by piercing or compressing a capsule containing the contrast agent, the capsule held in the channel until the contrast agent is dispensed.

FIGS. 14A-C illustrate an anchor that has not been optimally anchored. A gap remains between the tissue-facing surface of head 466 and a surface of the tissue 8. When contrast agent 486 is dispensed via channel 488 and port 482 into hollow 484, lack of contact between the tissue-facing surface of head 466 and surface of the tissue 8 allows the contrast agent to leak out. Suboptimal anchoring may mean incomplete anchoring (e.g., as shown in FIGS. 14A-C) or crooked anchoring (e.g., such that the head of the anchor does not lie flat against the tissue). For some applications, suboptimal anchoring may be due to anchoring into incorrect tissue, e.g., if tissue-engaging element 462 hits a hard area of tissue the anchor may not be fully anchorable. Suboptimal anchoring may be identified by fluoroscopic observation of the contrast agent leaking into (e.g., “puffing” within) the bloodstream, and/or fluoroscopic observation of the quantity of the contrast agent within hollow 484 becoming reduced.

In FIGS. 14D-F, tissue anchor 420 has been optimally anchored, such that the tissue-facing surface of head 466 is pressed against (e.g., sealed against) the surface of tissue 8. This optimal anchoring may be achieved simply by driving tissue-engaging element 462 deeper into the tissue (e.g., FIGS. 14D-F can represent subsequent steps immediately following the steps of FIGS. 14A-C), or can be achieved by de-anchoring, repositioning, and re-anchoring the anchor.

When contrast agent 486 is dispensed, e.g., injected, into hollow 484 after optimal/complete tissue anchor implantation, the contrast agent is better retained within hollow 484 of the head 466, as shown in FIGS. 14E-F. For example, the contrast agent may remain within the hollow for a limited span of time after dispensing. Optimal/complete anchoring may be identified by fluoroscopic observation of a lack/absence of the contrast agent leaking into (e.g., “puffing” within) the bloodstream, and/or fluoroscopic observation of the contrast agent remaining within hollow 484. After confirmation of optimal/complete placement, driver 487 can be removed (FIG. 14F).

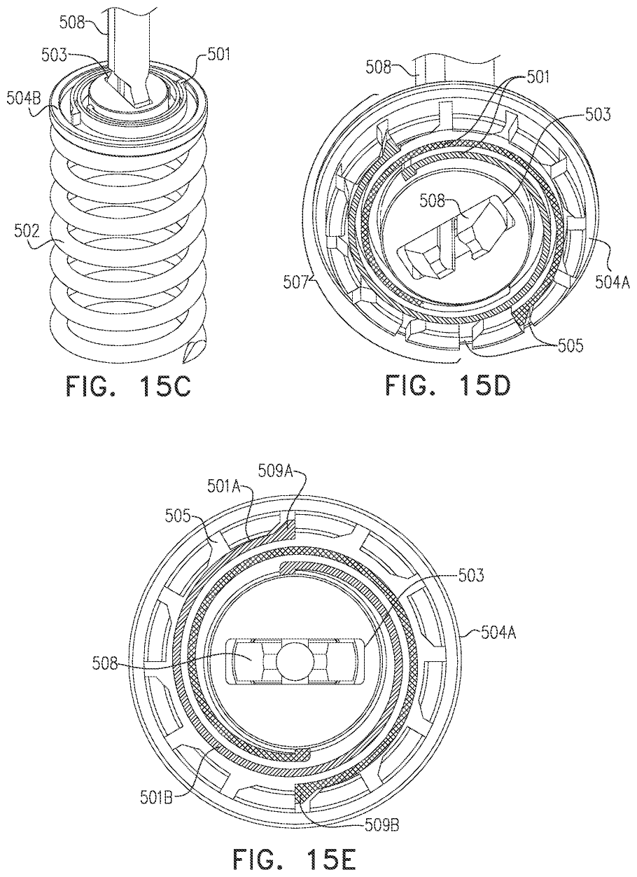

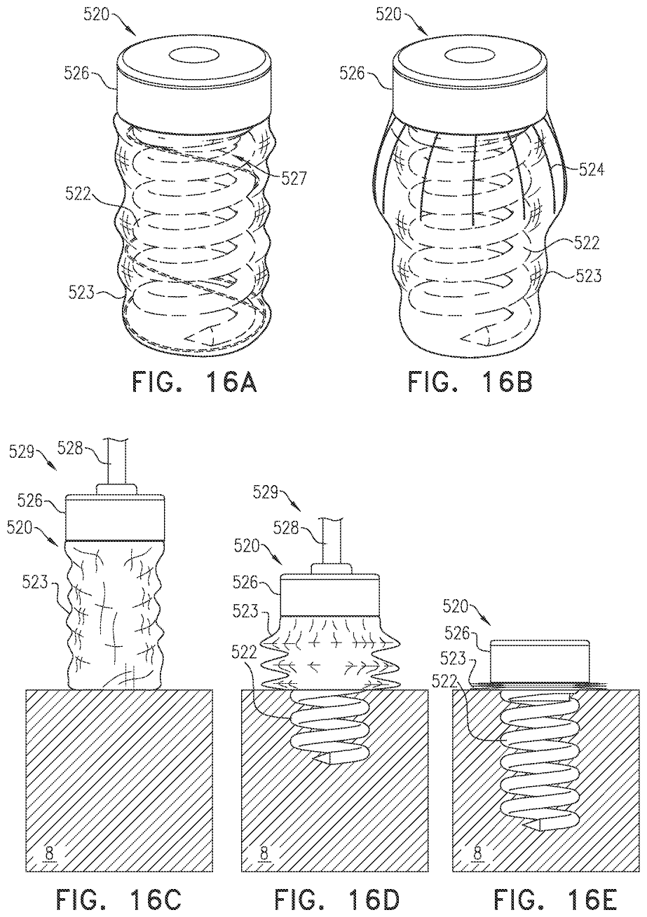

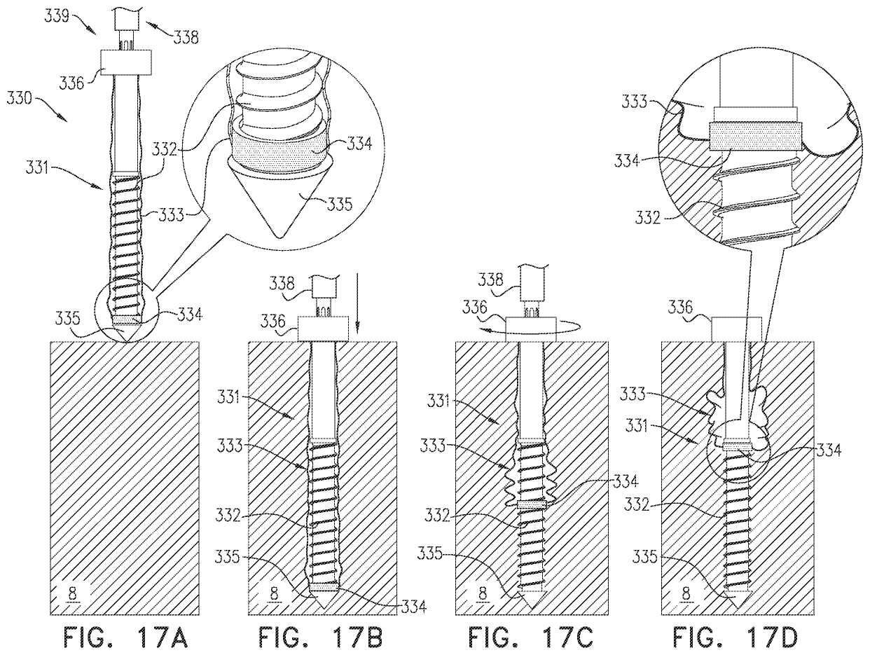

Reference is made to FIGS. 15A-E, which are schematic illustrations of a system 500 comprising a tissue anchor 510, in accordance with some applications. Tissue anchor 510 comprises a head 506, and a tissue-engaging element 502. Head 506 can comprise a base 504 (e.g., comprising a first base component 504a and a second base component 504b). Head 506 typically comprises an interface 503, and a torque limiter 507, operatively coupling the interface to the tissue-engaging element. Tissue-engaging element 502 can be a helical tissue-engaging element extending distally from the head and have a sharp distal tip.

For some applications, and as shown, torque limiter 507 and/or components thereof can be disposed internally with respect to head 506 (e.g., within base 504). For some applications, the torque limiter and/or components thereof can be disposed axially between interface 503 and tissue-engaging element 502. For some applications, the torque limiter and/or components thereof can be disposed proximally from base 504.

Apparatus 500 can further comprise a driver 508, configured to reversibly engage interface 503 and to screw tissue-engaging element 502 into the tissue by applying torque to the interface. Interface 503 can be disposed between base component 504a and base component 504b. For some applications, driver 508 can enter anchor head 506 in order to reversibly engage interface 503.

The torque limiter can comprise a variety of mechanisms, such as a pawl-and-spring mechanism defining a slip clutch, a gear set, a ball-detent mechanism, or a shear pin.

For some applications, e.g., that shown in FIGS. 15A-E, the torque limiter comprises a slip clutch that comprises a torsion spring 501 having a pawl 509 at its outer extremity. More specifically, two intertwined torsion springs (501a, 501b) can be configured in a double spiral lying on a spring plane, each with a respective pawl (509a, 509b)—e.g., with the two pawls being opposite each other. Pawls 509a and 509b can engage an inner surface of base 504—e.g., notches 505 defined by the base. In the example shown, notches 505 are defined by base component 504a. As shown, notches 505 can lie in the same plane as springs 501a and 501b and/or pawls 509a and 509b, and moreover can circumscribe the springs. Base component 504a can be fixedly coupled (e.g., welded) to base component 504b, with the pawl-and-spring mechanism disposed between base components 504a and 504b, i.e., within a hollow defined by base 504. Base 504 (e.g., base component 504b thereof) can be fixedly coupled (e.g., welded) to tissue-engaging element 502.

FIG. 15A is a perspective view of system 500. FIG. 15B is an exploded view of anchor 510. FIG. 15C is a perspective view of system 500 with base component 504a removed in order to show engagement of interface 503 by driver 508. FIGS. 15D-E also show engagement of interface 503 by driver 508, but from a distal side of head 506, with base component 504b removed.

Driver 508 is configured to screw tissue-engaging element 502 into the tissue 8 by applying torque to interface 503. This torque is transferred via torque limiter 507 to tissue-engaging element 502. Toque limiter is configured to transfer this torque only up to a predefined threshold torque. That is, the torque limiter can be configured to limit the amount of torque that can be applied to the tissue-engaging element to a predetermined threshold, e.g., by slipping when the threshold is exceeded. As shown in FIG. 15E, as driver 508 rotates base 504a via interface 503, base component 504b and tissue-engaging element 502 rotate concurrently. When the predefined torque threshold is reached, pawls 509a, 509b on springs 501a and 501b, respectively, slip with respect to notches 505 on base component 504a, uncoupling the rotation of base component 504b (and tissue-engaging element 502) from rotation of base component 504a.

Torque limiter 507 can be operational in a single direction (e.g., for screwing in the anchor)—e.g., such that it doesn't limit the torque applied in the other direction (e.g., for unscrewing the anchor). For some applications, a different (e.g., greater) threshold for the torque limiter can be provided for the unscrewing direction. For some applications, driver 508 is configured to unscrew tissue-engaging element 502 from tissue 8 by applying reverse torque to interface 503. Torque limiter 507 can be configured to transfer the reverse torque to tissue-engaging element 502 even upon the reverse torque exceeding the predefined threshold torque.

The torque can advantageously be applied to the distal end of anchor 510, i.e., to tissue-engaging element 502. Placing the torque limiter within anchor head 506 can advantageously allow the application of torque to the distal-most component of the anchor. In previous tools, a torque limiter can be disposed on a proximal part of the tool (e.g., on the handle), and therefore respond to torque at the proximal end of the tool, which can be different to torque at the distal end of the tissue-engaging element or anchor.

It is to be noted that torque limiter 507 can be used with (e.g., positioned within) another anchor head (e.g., within a housing of another anchor head), mutatis mutandis. For example, torque limiter 507 can be included in the housing of downstream assembly 700, 700a, or 700b described in International (PCT) Patent Application Publication WO 2022/101817 to Tennenbaum et al., which is incorporated herein by reference. In such applications, the torque limiter can be disposed axially between a winch component (e.g., a spool) and the tissue-engaging element. Placing the torque limiter within an existing component can advantageously avoid a need to enlarge the anchor head despite the addition of the torque limiter.

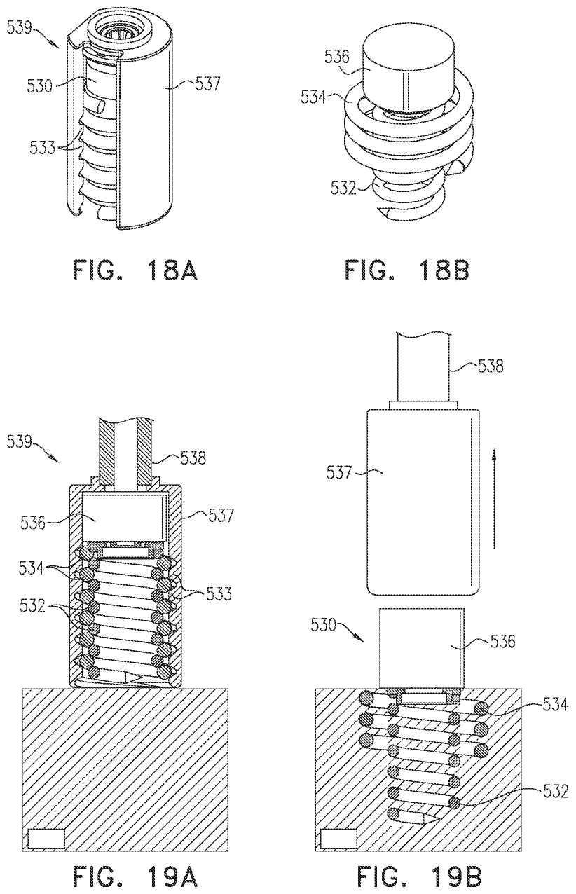

Reference is made to FIGS. 16A-E, which are schematic illustrations of a tissue anchor 520 having a head 526 and a helical tissue-engaging element 522, in accordance with some applications. Anchor 520 can be advanced and/or anchored using a driver 528.

For some applications, and as shown, anchor 520 further comprises a flexible sleeve 523, which can be tubular, and which extends distally away from head 526. For example, and as shown, sleeve 523 can extend over (e.g., coaxially surrounding) tissue-engaging element 522. For some applications, the proximal end of sleeve 523 can be rotatably coupled to head 526 of tissue anchor 520. For some applications, the distal end of sleeve 523 can be open. In some other applications, the end of the sleeve can be closed.

Sleeve 523 can be formed from a sheet that can comprise a mesh, a braid, a stretchable (e.g., woven or knitted) fabric, or a flexible film. For some applications, the sheet can be porous e.g., having pores with a pore-size greater than 75 microns. Such openings can facilitate migration and ingrowth of fibroblasts after tissue insertion of the anchor. The material composition of the mesh or braid can be polymer based, or metallic (e.g., nitinol). For some applications, the distal and proximal parts of sleeve 523 can comprise different materials and/or thicknesses, these differences in material properties contributing to the extent and rate of sleeve collapse during deployment, as further described herewithin below.

Sleeve 523 can be radiopaque and/or echogenic, thus allowing visualization of anchor 520 during fluoroscopy or other mid-procedure imaging. A radiopaque composition of the sleeve can advantageously facilitate both initial positioning and, based on its axial compression during anchoring, determination of anchoring depth.

For some applications, e.g., as shown in FIG. 16A, sleeve 523 can further comprise a flexible wire 527 extending helically along the sheet of the sleeve. For example, wire 527 can serve as a helical spring element configured to provide structure and elastic axial compressibility to sleeve 523. Wire 527 can be formed from a metal and/or a polymer. Wire 527 can be radiopaque and/or echogenic, thus providing an indication of a degree of tissue anchor deployment under fluoroscopy.

FIG. 16B shows an anchor 520a, which is a variant of anchor 520, which further comprises short prongs 524 (e.g., shorter than tissue-engaging element 522) extending distally from anchor head 526. Prongs 524 can be covered by sleeve 523, and/or can axially surround helical tissue-engaging element 522. That is, prongs 524 can be disposed laterally from tissue-engaging element 522, and medially from the sheet of sleeve 523.

FIG. 16B shows anchor 520a being absent of wire 527. For some applications, an anchor can comprise both wire 527 and prongs 524. For some applications, an anchor can comprise neither wire 527 nor prongs 524.

Prongs 524 can comprise barbs, spikes, and/or metal wires. Prongs 524 can be rotatably coupled to head 526 e.g., via a collar (not shown)—e.g., disposed axially between head 526 and tissue-engaging element 522.

Reference is made to FIGS. 16C-E, showing the process of driving the anchor into the tissue. As driver 528 advances anchor 520 into a tissue 8, sleeve 523 becomes axially compressed. Sleeve 523 can also concurrently expand radially outward during axial compression. For some applications, the distal end of sleeve 523 is closed, such that the tissue-engaging element can penetrate the distal end of the sleeve as the tissue-engaging element advances into the tissue.

In applications in which prongs 524 are present, sleeve 523 can initially shield prongs 524 from penetrating the tissue—e.g., prongs can become exposed (e.g., through the distal end of the sleeve, and/or by penetrating the sheet of the sleeve) only at a certain depth of anchoring. For some applications, control of the amount of prong exposure can be achieved by configuring the axial compressibility of sleeve 523 (e.g., the spring constant of wire 527), and/or by a proximal part of the sleeve comprising a different material from a distal part of the sleeve. For some applications, when tissue-engaging element 522 is fully inserted into tissue 8, prongs 524 can also penetrate into the tissue, advantageously providing further anchoring strength and/or helping to prevent tissue-engaging element 522 from unintentional unscrewing.

Prior to insertion of anchor 520 (e.g., during advancement of the anchor through the vasculature), sleeve 523 can entirely cover tissue-engaging element 522. This feature can advantageously shield tissue-engaging element 522 (e.g., its sharp distal tip) from tissue and/or other implant elements and/or tools that can be simultaneously present—e.g., thereby reducing a chance of inadvertent snagging and/or damage.

For some applications, axial re-expansion of sleeve 523 can be facilitated by wire 527, such that sleeve 523 resumes its original configuration in the event that anchor 520 is unscrewed (e.g., in order to remove the anchor from the subject).

For applications in which sleeve 523 is radiopaque, an ability to visualize the sleeve on fluoroscopy during placement can facilitate both initial positioning and, based on the amount of axial compression during anchoring, determination of anchoring depth. It is hypothesized that sleeve 523 can advantageously improve initial anchoring—e.g., by acting as a spring washer, and thus reducing the likelihood of unintentional loosening of tissue anchor 520.

In applications in which sleeve 523 comprises a mesh or porous material, its pores can advantageously promote long-term anchoring by facilitating tissue growth therethrough. It is expected that tissue ingrowth will allow complete incorporation of the anchor within the surrounding tissue within two weeks after insertion, thereby assisting to stabilize the anchor in the tissue.

A similar anchor can be provided in which a proximal part of the sleeve can be flexible, as described herewithin above, and a distal portion of sleeve can be rigid. The distal portion can have an inner thread, such that tissue-engaging element can be screwed along the thread and into the tissue. As the tissue-engaging element is screwed into the tissue, the proximal portion of the sleeve can become compressed between the anchor head and the tissue-engaging element, e.g., as shown in FIGS. 16C-E.

Reference is made to FIGS. 17A-D, which are schematic illustrations of a tissue anchor 330 having a head 336 and a tissue-engaging element 331 comprising a threaded shaft 332 having a sharp distal tip 335, in accordance with some applications. Anchor 330 can further comprise a flexible sleeve 333, which can be tubular, and which extends distally away from head 336—e.g., coaxially surrounding shaft 332. Anchor 330 can be advanced and/or anchored using a driver 338 of a delivery tool. Thus, a system 339 can be provided, comprising anchor 330 and the delivery tool that comprises driver 338.

For some applications, and as shown, the distal end of sleeve 333 can be fitted with a threaded ring 334 having a complementary pitch and handedness to the threads on shaft 332. In a delivery state, sleeve 333 can be secured to shaft 332 near distal tip 335 via engagement between threaded ring 334 and the threads of the shaft, as shown in FIG. 17A.

Sleeve 333 can be formed from a sheet that can comprise a mesh, a braid, a stretchable (e.g., woven or knitted) fabric, or a flexible film. For some applications, the sheet can be porous e.g., having pores with a pore-size greater than 75 microns. Such openings can facilitate migration and ingrowth of fibroblasts after tissue insertion of the anchor. The material composition of the mesh or braid can be polymer based, or metallic (e.g., nitinol). For some applications, the distal and proximal parts of sleeve 333 can comprise different materials and/or thicknesses, these differences in material properties contributing to the behavior of sleeve 333 during anchoring, as further described herewithin below.

Sleeve 333 and/or ring 334 can be radiopaque and/or echogenic, thus allowing visualization of anchor 330 during fluoroscopy or other mid-procedure imaging. A radiopaque composition of the sleeve can advantageously facilitate both initial positioning and, based on its axial compression during anchoring, determination of anchoring depth.

For some applications, and as shown in FIGS. 17A-D, driver 338 advances tissue-engaging element 331 into a tissue 8 linearly (e.g., without the need for rotation/screwing), with sleeve 333 advancing concurrently with shaft 332—e.g., because the distal end of the sleeve is prevented from slipping along the shaft by the engagement between ring 334 and the threads of the shaft. Once tissue-engaging element 331 has reached its full depth within tissue 8, driver 338 rotates shaft 332 by applying torque to head 336. Friction between tissue 8 and sleeve 333 and/or ring 334 inhibits the sleeve and/or the shaft from rotating, such that the rotation of shaft 332 is with respect to ring 334, and thereby draws the ring proximally along the threads of the shaft. As ring 334 moves proximally, a distal portion of sleeve 333 responsively expands radially outward (FIG. 17C). At a point at which ring 334 reaches a proximal limit of threads on shaft 332, the ring stops in its proximal retraction (FIG. 17D). The expanded sleeve may provide immediate anchoring due to mechanical interaction (e.g., pressure, friction) with the tissue, and may subsequently provide additional anchor via tissue growth as described hereinabove. Driver 338 may then disengage from ring 334 and uncouple from anchor 330.

Reference is made to FIGS. 18A-B and 19A-B, which are schematic illustrations of a system 539 comprising a tissue anchor 530 and a driver 538, in accordance with some applications. Tissue anchor 530 comprises a head 536, a first helical tissue-engaging element 532, and a second helical tissue-engaging element 534. System 539 further comprises a removable delivery capsule 537, within which anchor 530 may be disposed prior to deployment—e.g., during advancement through the vasculature. Anchor 530 can be advanced and/or anchored into the tissue using driver 538.

For some applications, and as shown, tissue-engaging element 532 and tissue-engaging element 534 are coaxial and extend distally from head 536. Each tissue-engaging element is coupled to head 536 and ends in a sharp distal tip. First tissue-engaging element 532 may be disposed medially to second tissue-engaging element 534—e.g., as shown.

Tissue-engaging element 534 can comprise an elastic, e.g., shape memory material, such that it is compressible inside of capsule 537 prior to deployment, and is configured, upon deployment, to expand radially to assume a larger diameter, e.g., to a diameter greater than the diameter of head 536, and/or greater than the diameter of tissue-engaging element 532.

For some applications, tissue-engaging element 532 can comprise a non-compressible/non-expandable material, e.g., stainless steel or titanium.

For some applications, tissue-engaging element 532 can comprise a compressible material, such that it too is expandable upon deployment. In such applications, second (outer) tissue-engaging element 534 typically expands to a greater diameter than first (inner) tissue-engaging element 532.

Prior to delivery, anchor 530 (i.e., head 536 and both tissue-engaging elements 532 and 534) is configured to be disposed inside delivery capsule 537. In the pre-deployment configuration, both tissue-engaging elements 532, 534 typically have the same or similar diameters within capsule 537. Driver 538 is configured to drive (e.g., screw) anchor 530 distally out of capsule 537, such that second helical tissue-engaging element 534 expands radially outward. For some applications, this expansion may occur as one or both tissue-engaging elements are advanced into a tissue 8. Thus, upon full deployment, tissue-engaging element 534 has a larger diameter than tissue-engaging element 532.

For some applications, and as shown, capsule 537 can have internal threads 533. One or both of first tissue-engaging element 532 and second tissue-engaging element 534 can travel along the internal threads 533 during deployment.

An anchor comprising a second tissue-engaging element, such as anchor 530, may advantageously provide improved tissue contact and greater anchoring strength. Anchor 530 is configured to have an increased anchoring surface compared to a tissue anchor having a single helical tissue-engaging element. Arranging the second coaxial helical tissue-engaging element in an expandable configuration may enable additional holding force without requiring a larger pre-deployment diameter of the anchor.

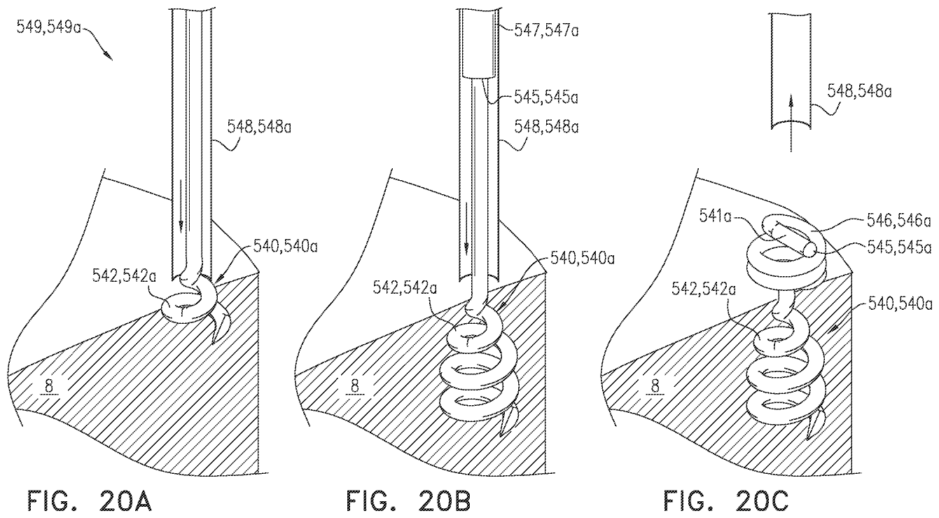

Reference is made to FIGS. 20A-C and 21A-C, which are schematic illustrations of variants of a system 549 comprising a tissue anchor 540 and a delivery tool 548, in accordance with some applications.

For some applications, and as shown, delivery tool 548 comprises a driver 547, configured to advance anchor 540 into the tissue.

For some applications, anchor 540 (e.g., head 546 and/or tissue-engaging element 542) comprises a shape memory material, e.g., nitinol. For some applications, and as shown, a single length of wire can be shaped to define both head 546 (546a, 546b) and tissue-engaging element 542 (542a, 542b).

FIGS. 20A-C show a system 549a, which is a variant of system 549 comprising a tissue anchor 540a (which is a variant of tissue anchor 540) and a delivery tool 548a (which is a variant of delivery tool 548).

As shown, anchor 540a comprises a head 546a and a helical tissue-engaging element 542a, both of which can be expandable.

For delivery, anchor 540a can be disposed within delivery tool 548a, comprising a tube, and having an internal diameter that is smaller than the deployed width of anchor head 546a. That is, anchor 540 is constrained linearly within the tube of the delivery tool.

When delivery tool 548a reaches a surface of a tissue 8 into which anchor 540a is to be advanced, driver 547a is configured to advance anchor 540a into tissue 8 by pushing on proximal end 545a of the anchor. As driver 547a pushes anchor 540 into the tissue, strain stored in the elongated form of the anchor can be released, thus enabling the tissue-engaging element to advance forward into the tissue. As tissue-engaging element 542a exits delivery tool 548a, the tissue-engaging element assumes its original shape, i.e., the memory shape, within the tissue. For some applications, and as shown, the memory shape is helical. Thus, for such applications, a helical tissue-engaging element 542a is driven (e.g., screwed) into the tissue solely by being driven axially out of delivery tool 548a (e.g., the tube thereof).

Driver 547a can be configured to stop pushing on proximal end 545a once tissue-engaging element 542a has been fully deployed and anchored in the tissue (FIG. 20B). For example, the driver may be limited in how far distally it can push the anchor out of the delivery tool. At this point of the anchor deployment procedure, delivery tool 548a can be withdrawn proximally. As the delivery tool retracts proximally, head 546a becomes exposed from delivery tool 548a, and responsively assumes a deployed shape in which it rests on the surface of tissue 8 (FIG. 20C). For example, and as shown, in its deployed shape, head 546a can coil up to provide a tissue-contact surface analogous to that of other tissue anchors. In the example shown, head 546 is helical and has the same handedness as the helix defined by tissue-engaging element 542a. However, for some applications, head 546 can have an opposite handedness to tissue-engaging element 542a.

For some applications, anchor 540a can be retrievable. For some applications, retrieval can be achieved by delivery tool 548a (e.g., driver 547a thereof) pulling proximal end 545a, followed by the rest of the anchor including tissue-engaging element 542a, back into the delivery tool, with the anchor responsively re-assuming its elongated shape. For some applications, head 546 (e.g., once formed) can comprise a crossbar 541a configured to be grasped by a discrete retrieval tool that can retrieve the anchor by pulling and/or by unscrewing the anchor (e.g., by applying torque to the crossbar). Alternatively, or additionally, crossbar 541a can be used to facilitate additional tightening of anchor 540a to the tissue. For some applications, crossbar 541a can be used to couple another component to anchor 540a intracorporeally.

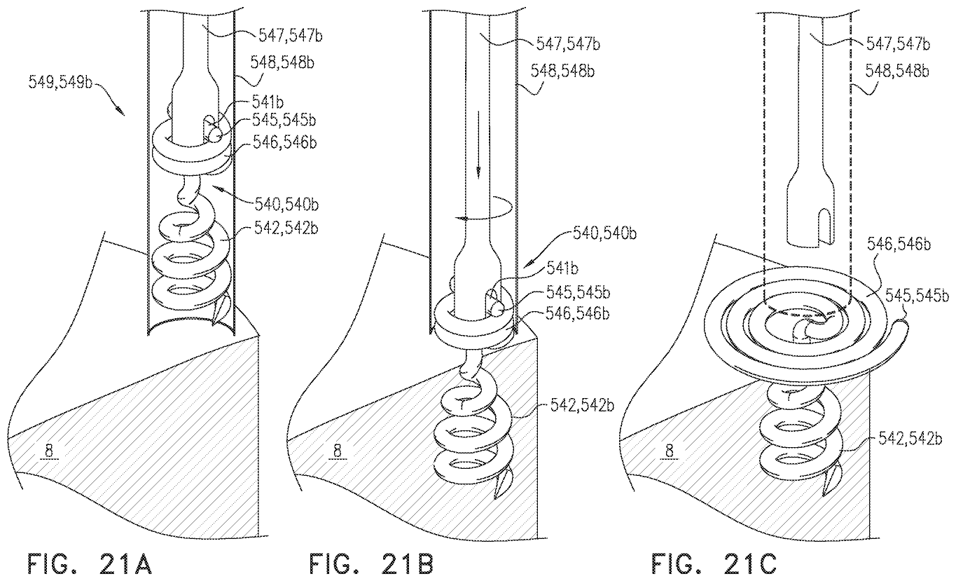

FIGS. 21A-C show a system 549b, which is a variant of system 549, comprising a tissue anchor 540b (which is a variant of tissue anchor 540) and a delivery tool 548b (which is a variant of delivery tool 548).

For some applications, and as shown, anchor 540b comprises a head 546b, typically expandable, and a helical tissue-engaging element 542b, which may or may not be expandable.

For delivery, anchor 540b can be disposed within delivery tool 548b, comprising a tube, and having an internal diameter that is smaller than the deployed width of anchor head 546b. Thus, anchor 540b is delivered while in a delivery state in which head 546b is constrained to a diameter smaller than the internal diameter of delivery tool 548b.

When delivery tool 548b, engaged with proximal end 545b of anchor 540b, reaches a surface of a tissue 8 into which the anchor is to be advanced (FIG. 21A), driver 547b is configured to drive tissue-engaging element 542b into the tissue by applying torque to the proximal end of the anchor (FIG. 21B). Upon head 546b exiting delivery tool 548b, the head is configured to transition toward its resting shape, i.e., the memory shape (FIG. 21C). For some applications, and as shown, the memory shape of the head is a spiral having a diameter that is at least 50 percent wider than (e.g., at least twice as wide as, such as at least three times as wide as) the outer diameter of driver 547b. For some applications, and as shown, the diameter of head 546b in its memory shape can be at least twice (e.g., at least three times) that of tissue-engaging element 542b.

When tissue-engaging element 542b has fully deployed and anchored in the tissue, driver 547b can be disengaged from proximal end 545b (FIG. 21C). For some applications, the shape-transitioning of head 546b may automatically disengage the head from driver 547b. At this point of the anchor deployment procedure, delivery tool 548b can be withdrawn.

For some applications, and as shown, in the delivery state of anchor 540b, head 546b can define a crossbar 541b configured to be engaged by driver 547b, and via which torque is applied to the anchor. In some such applications, such as shown in FIGS. 21A-C, crossbar 541b is present as a distinct feature only in the delivery state, e.g., can assume a shape continuous with the distal region of the head upon deployment.

Typically, tissue anchors, e.g., those that traverse the vascular system, are limited by a diameter of the delivery system. The delivery system diameter is in turn limited by the diameters of the smaller blood vessels of the subject through which the delivery system is configured to be advanced. This size limitation thus constrains either the size of the anchor head, and/or the size of the vessel into which the anchor can be advanced. Tissue anchor 540, and variants 540a and 540b thereof, may advantageously enable the delivery tool to traverse blood vessels having an internal diameter less than the diameter of a head of a typical tissue anchor, and less than the deployed diameter of head 546.

Further advantageous features of anchor 540 (540a, 540b) include a low protrusion of the helical coil comprising head 546 (546a, 546b) above the surface of tissue 8, which may reduce a likelihood of thrombus formation at the head, and/or may reduce turbulence of blood flowing past the head. For some applications, tissue ingrowth may occur at an accelerated rate compared to a standard tissue anchor, due to the low protrusion of head 546 above the surface of tissue 8, resulting in favorable tissue encapsulation. Anchor 540 may further advantageously reduce a likelihood of implant dislodgement by reducing a likelihood of head 540 pulling through an implant or tissue that it is intended to anchor.

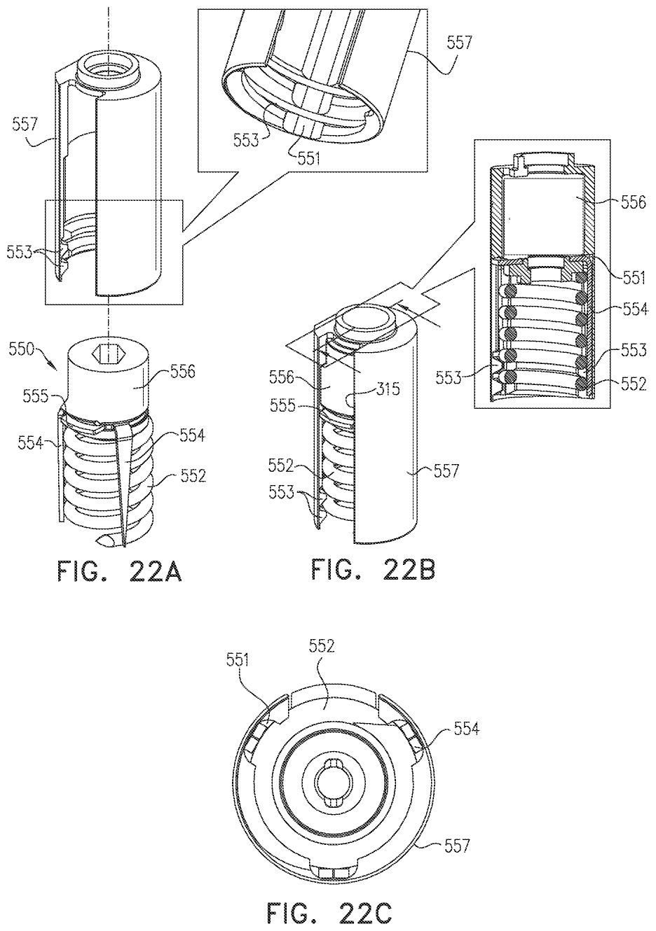

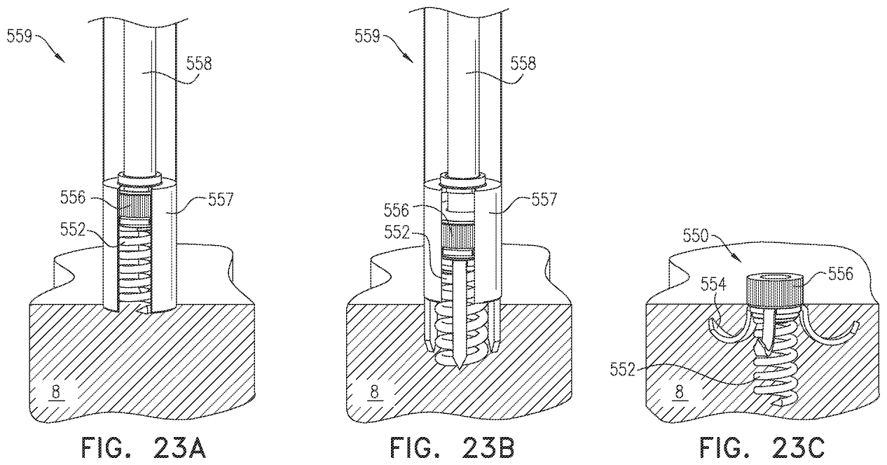

Reference is made to FIGS. 22A-C and 23A-C, which are schematic illustrations of a system 559 comprising a tissue anchor 550, in accordance with some applications.

FIGS. 22A-C and 23A-C show a system 559, comprising a tissue anchor 550, and a delivery tool comprising a driver 558 and a sheath 557.

For some applications, and as shown, anchor 550 comprises a head 556 and a first helical tissue-engaging element 552. First tissue-engaging element 552 can be rotationally fixed with respect to head 556 (e.g., an interface thereof). Anchor 550 further comprises a second tissue-engaging element 554 comprising a set of radially-expandable prongs coupled to head 556, e.g., by a collar, and extending distally therefrom. The prongs of tissue-engaging element 554 can be rotationally coupled to head 556 and/or first tissue-engaging element 552, and can be disposed laterally, e.g., circumferentially, thereto. The prongs of second tissue-engaging element 554 can comprise, e.g., barbs, spikes, and/or metal wires. Prongs of tissue-engaging element 554 can comprise a memory shape material such as nitinol. The prongs can have a preset memory shape, such that each prong is biased to extend laterally, such as in a curve—e.g., in a shape of a proximally-directed hook.

Sheath 557 can comprise a cylindrical capsule, the sheath having internal helical threads 553 that match the pitch and handedness of the first tissue-engaging element 552, such that tissue-engaging element 552 fits rotatably within the sheath. Sheath 557 can further comprise longitudinal, i.e., axial, grooves 551, sized and positioned complementarily to fit the prongs of tissue-engaging element 554.

Sheath 557 can further comprise a longitudinal slit 315, extending axially along the sheath. Second tissue-engaging element 554 (e.g., the collar thereof) can comprise or define a tongue 555 that protrudes into and/or through slit 315, and that is configured to maintain the second tissue-engaging element rotationally stationary during rotation of first tissue-engaging element 552.

For some applications, and as shown, e.g., in the inset of FIG. 22A, axial grooves 551 can intersect helical threads 553. For some applications, and as shown, axial/linear grooves 551 can extend deeper into the wall of sheath 557 than does helical threads 553. Helical threads 553 can prevent premature deployment of second tissue-engaging element 554—e.g., by preventing the shape memory of the prongs from pushing backwards on the distal rim of sheath 557, pulling the anchor out of the capsule before tissue-engaging elements 552 and 554 have been driven into the tissue.

For delivery, anchor 550 can be disposed within the delivery tool, specifically within sheath 557, such that first and second tissue-engaging elements 552, 554 are constrained within the sheath (FIG. 23A). Upon reaching the surface of tissue 8, driver 558 is used to screw tissue-engaging element 552 into the tissue (FIG. 23B). As tissue-engaging element 552 advances helically along thread 553, it progressively exits sheath 557 into tissue 8. Concurrently, the prongs of tissue-engaging element 554 advance axially into the tissue, i.e., without rotation, along axial grooves 551. Whereas axial advancement of second tissue-engaging element 554 is coupled to advancement of first helical tissue-engaging element 552, sheath 557 (e.g., axial grooves 551 and longitudinal slit 315 thereof) inhibits rotation of tissue-engaging element 554, such that tissue-engaging element 552 rotates independently.

Screwing of helical tissue-engaging element 552 into tissue 8 is configured to pull anchor 550, including tissue-engaging element 554, out of sheath 557. As the prongs of tissue-engaging element 554 are released from the sheath, they expand radially within the tissue.

An anchor comprising a second tissue-engaging element, such as anchor 550, may advantageously provide improved tissue contact and greater anchoring strength, compared to anchors that have only a single (e.g., helical) tissue-engaging element. Further advantageous features of anchor 550 concern the prongs, which are hypothesized to reduce the chance of undesired unscrewing of the anchor from the tissue, e.g., by generating axial force pulling the anchor head and helix against the tissue, thereby increasing friction and preventing helical tissue-engaging element 552 from rotating outwards from the tissue. Also advantageously, when deployed, anchor 550 engages a tissue perimeter larger than the diameter of the catheter.

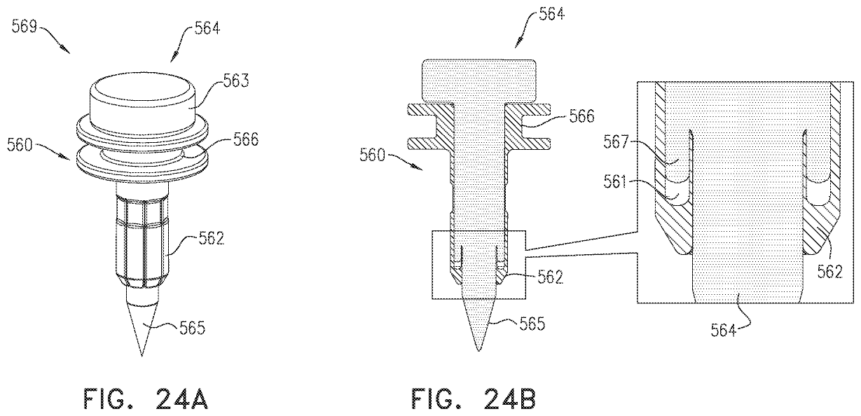

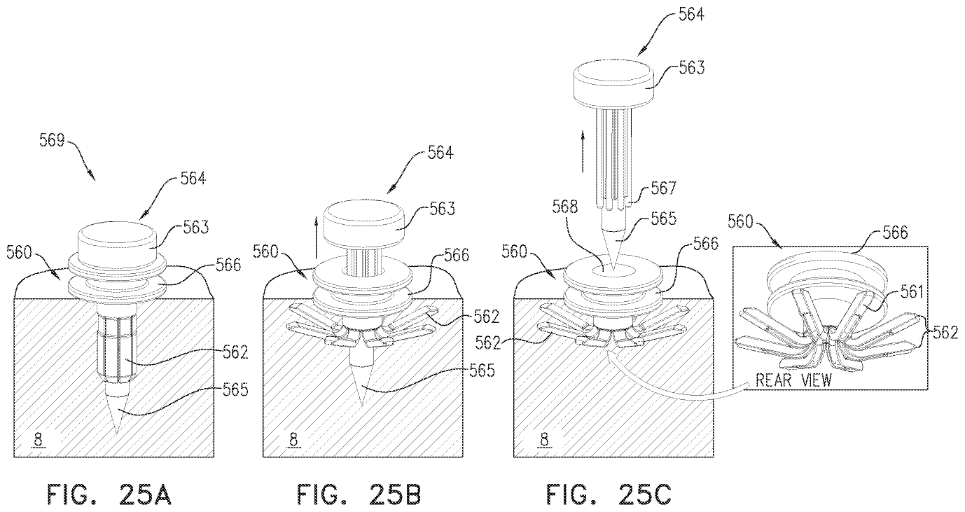

Reference is made to FIGS. 24A-B and 25A-C, which are schematic illustrations of a system 569 comprising a tissue anchor 560, in accordance with some applications.

For some applications, and as shown, anchor 560 comprises a head 566 and a set of tissue-engaging prongs 562. Head 566 defines an aperture 568 extending therethrough along a longitudinal axis of the anchor. Tissue-engaging prongs 562 can be arranged circumferentially around the longitudinal axis of anchor 560. Each prong 562 can define a pocket 561, typically at a distal part of the prong (FIG. 24B insert). Anchor 560, e.g., tissue-engaging prongs 562, can comprise nitinol or another memory shape material. In some such applications, prongs 562 can be shape set to an expanded, i.e., deployed, shape. For some applications, and as shown (FIG. 24A), anchor 560 is formed from a single cylindrically-shaped element, in which head 566 is defined by the proximal end of the cylindrically-shaped element, and the distal section of the cylindrically-shaped element is cut lengthwise into a set of (e.g., four, e.g., six, e.g., eight) prongs. These prongs can then be bent outwards and shape set into a splayed, i.e., deployed, configuration.

System 569 further comprises an insert 564, configured to fit within and extend through aperture 568 in head 566. For some applications, and as shown, insert 564 has a proximal head region 563. For some applications, and as shown, insert 564 has a sharpened distal tip 565. Insert 564 can comprise a set of fingers 567 corresponding to the set of tissue-engaging prongs. Fingers 567 are dimensioned to be secured to anchor 560 by insert 564 extending through aperture 568 such that fingers 567 are disposed within pockets 561 in a manner that constrains anchor 560 in a delivery state in which prongs 562 are disposed medially compared to in their deployed state. For some applications, in the delivery state of anchor 560, prongs 562 are substantially parallel with each other and/or with the longitudinal axis of the anchor. For some applications, and as shown, fingers 567 are parallel to each other.

For some applications, during assembly of system 569, insert 564 can be inserted into aperture 568, while prongs 562 are constrained medially (e.g., by an external force), such that fingers 567 slides into pockets 561.

Before deployment into a tissue 8, and as shown (FIGS. 24A-B), prongs 562 are constrained medially (e.g., substantially parallel to the longitudinal axis), to assume the delivery state. During anchor deployment, system 569 can be pushed axially into tissue 8 (FIG. 25A). For applications in which insert 564 has sharpened distal tip 565, the sharpened distal tip pierces the tissue during this pushing. Upon anchor 560 reaching a desired depth, insert 564 can be intracorporeally retracted (e.g., pulled proximally) to withdraw fingers 567 from pockets 561, thereby releasing prongs 562 to responsively move laterally toward their deployed state (FIG. 25B).

For some applications, insert 564 is completely removed from anchor 560—e.g., as shown in FIG. 25C. For some such applications, system 569 can comprise an additional component or feature to seal aperture 568 in head 566 upon or after removal of insert 564.

For some applications, insert 564 remains coupled to anchor 560, e.g., may be retracted only by a distance sufficient such that fingers 567 disengage from pockets 561, and such that prongs 562 assume the deployed memory shape. For example, insert 564 may always remain disposed through aperture 568. For such applications, FIG. 25B may represent the extent to which insert 564 is retracted. For some such applications, insert 564 remains in this retracted position (e.g., FIG. 25B represents a final state of system 569). For other such applications, following its retraction, insert 564 can be readvanced distally into head 566 (e.g., FIG. 25B represents an intermediate state of system 569)—although pockets 561 would not become reengaged by fingers 567, and so prongs 562 would remain protruding laterally.

An anchor comprising multiple tissue-engaging elements, such as anchor 560, may advantageously provide improved tissue contact and greater anchoring strength, compared to anchors having only a single (e.g., helical) tissue-engaging element. Such an anchor may advantageously be stronger and more resistant to pullout, by distributing the force over a greater area of tissue. It may also be easier to deploy, as no rotation or tightening is needed. If necessary, the anchor may also be removed from the tissue and repositioned before the insert is removed. Removing and repositioning such an anchor would be easier than, e.g., an anchor having a helical tissue-engaging element configured to be screwed into the tissue. Also, advantageously, the tissue-engaging elements of anchor 560 lack sharp points on the medial/inner side that could damage tissue.

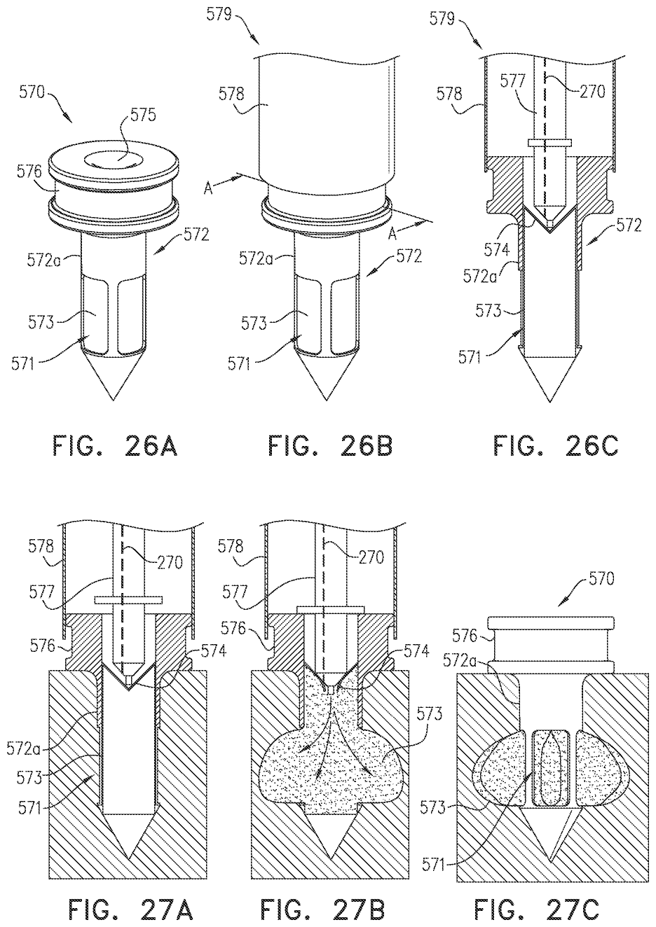

Reference is made to FIGS. 26A-C and 27A-C, which are schematic illustrations of a system 579 comprising a tissue anchor 570, in accordance with some applications.

For some applications, and as shown, anchor 570 comprises a head 576 and a tissue-engaging element 572. Head 576 can comprise an aperture 575 (e.g., a hollow), which can be fitted with a closure 574 near a junction of head 576 with tissue-engaging element 572.

For some applications, and as shown, tissue-engaging element 572 can comprise a shaft 572a having a longitudinal axis and a sharp distal tip to facilitate tissue penetration. For some applications, and as shown, shaft 572a is hollow. Shaft 572a can comprise or define a lateral wall having a series of windows 571 arranged circumferentially around the lateral wall. The series can comprise, e.g., two, e.g., three, e.g., four, e.g., five, e.g., six windows. Anchor 570 can further comprise an expandable balloon 573, e.g., one balloon, or a set of balloons corresponding to the series of windows, within the shaft of tissue-engaging element 572. Balloon 573 can extend to closure 574, such that the balloon occupies substantially the entire hollow shaft of tissue-engaging element 572.

System 579 can further comprise a delivery tool 578 comprising a balloon expander (e.g., inflator) 577. Balloon expander 577 can be configured to inject, via aperture 575 in head 576 through closure 574, a fluid into balloon 573. Closure 574 can comprise a one-way valve to retain the fluid in the balloon. Delivery tool 578, e.g., balloon expander 577, can further comprise a mechanism to override (e.g., traverse) the valve and withdraw the fluid if needed, e.g., to move anchor 570.

During delivery, delivery tool 578 is configured to advance anchor 570 into tissue 8. When the operator is satisfied with the location of anchor 570 in tissue 8, a fluid, e.g., a saline solution, can be injected via balloon expander 577 into anchor 570, filling balloon 573, and causing the balloon to expand radially, via windows 571, away from the lateral surface of tissue-engaging element 572 within tissue 8.

For some applications, the introduced fluid is substantially inert—e.g., saline. For some applications, the introduced fluid is configured to harden after its introduction —e.g., an epoxy or other resin. To allow for adjustment and repositioning, the hardening material can be configured to harden only upon the operator performing a hardening step, e.g., after the operator is satisfied with the position and state of the anchor in its inflated state. For example, the hardening material may require curing in order to harden, e.g., by exposure to ultraviolet light. For this purpose, a small optical fiber 270 integral to the delivery tool may be used to apply UV light to induce hardening of the adhesive, e.g., when the user is satisfied with the anchor location. The optical fiber of the delivery tool, if present, may have access to the epoxy in the anchor, e.g., via closure 574 or via a window near the closure. For some applications, and as shown, optical fiber 270 is coupled to balloon expander 577.

An anchor comprising inflatable, balloon-based tissue-engaging elements, such as anchor 570, may be advantageously effective in various applications, e.g., both those in which the anchor is configured to completely traverse thin tissue, and those in which the anchor is configured to be anchored into thicker tissue. A further advantageous feature of anchor 570 is the ability to expand the balloon laterally beyond the diameter of head 576, such that the anchor may advantageously be stronger and more resistant to pullout, by distributing the force over a greater area of tissue. A still further advantageous feature of anchor 570 is the ability to repeatedly reposition and/or reinsert the anchor into tissue (e.g., in a minimally traumatic manner) prior to its final securing by inflating balloon 573.

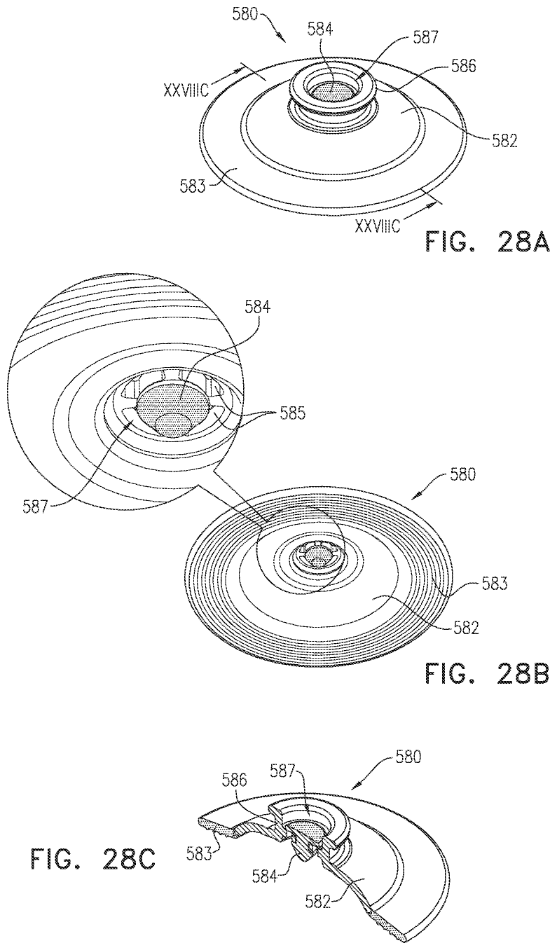

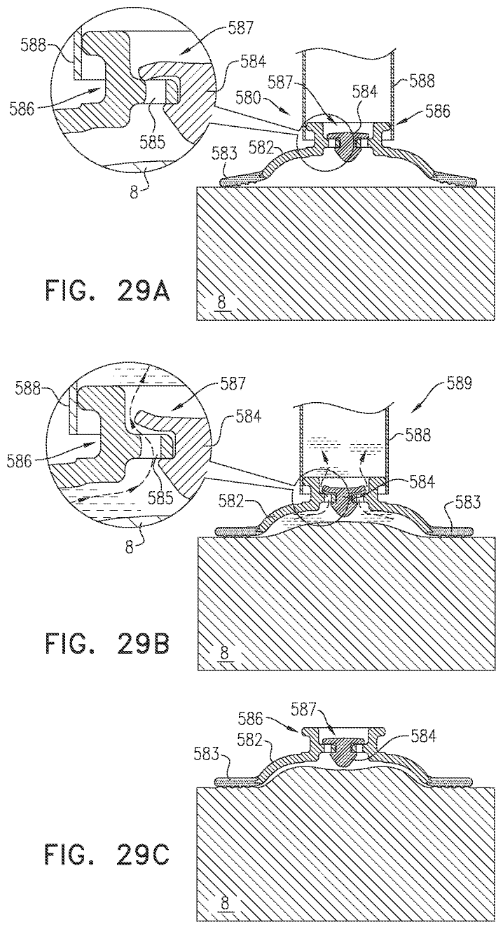

Reference is made to FIGS. 28A-B and 29A-C, which are schematic illustrations of a system 589 comprising a tissue anchor 580 and a delivery tool 588 therefor, in accordance with some applications.

For some applications, and as shown, anchor 580 comprises a head 586 and a cup 582, which is configured to act as a suction device. Head 586 can be engaged by delivery tool 588, for delivery of anchor 580. Head 586 has one or more ports 585 therethrough. Ports 585 are configured to provide axial fluid communication through head 586. A valve member 584 (e.g., a disc and/or membrane) can be disposed at ports 585, such that the ports and valve member 584 collectively define a valve 587, such as a check valve. In a resting state, valve member 584 can cover (e.g., seal) ports 585 (FIG. 29A). For example, and as shown in FIG. 29A, in the resting state of valve member 584, an exposed surface of the valve member can be flat or slightly convex.

For some applications, and as shown, valve member 584 can comprise a resilient stopper or plug that secures the valve member in place. The stopper, or valve member 584 as a whole, can comprise, e.g., silicone or another polymer.

Cup 582 has a flexible flange 583 that defines a rim of the cup. For some applications, more medial portions of the cup are rigid, at least relative to flange 583. Cup 582 can comprise, e.g., a metal or other non-flexible, biocompatible material. Flange 583 can comprise, e.g., silicone or another biocompatible polymer, configured to conform to the surface topography of a tissue to which anchor 580 is to be fixed, thereby creating a potential seal with the tissue. Thus, cup 582 is enabled to serve as a suction cup, to which suction can be applied via ports 585 (e.g., via valve 587) on head 586.

During delivery, anchor 580 can be introduced into a chamber of the heart—e.g., through a delivery tool. Anchor 580 is positioned for deployment by placing flange 583 against a surface of a tissue 8—e.g., on the annulus of a valve of a heart, or on a wall of the chamber of the heart. Reduced pressure is created between cup 582 and the surface of tissue 8, e.g., by applying suction at ports 585—e.g., at valve 587. Responsively, valve member 584 can flex away from ports 585 (e.g., such that its exposed surface becomes concave), thereby allowing fluid, e.g., blood, disposed between the cup and the tissue surface, to be drawn out via ports 585. This reduced pressure, along with the sealing between flange 583 and tissue 8, results in a holding force between cup 582 and tissue 8.

The holding force of anchor 580 may be dependent on the cross-sectional and/or surface area of the cup. For example, a substantially circular cup of diameter 3.2 mm would yield a holding force of at least approximately 0.0825 kg, and a circular cup of diameter 6.35 mm would yield a holding force of at least approximately 0.42 kg (calculated at atmospheric pressure; the holding force is predicted to increase with higher pressure, e.g., chamber pressure in a heart). Once tissue heals over the anchor, the holding force will increase significantly and may no longer rely on the vacuum.

Anchor 580 advantageously provides a surface anchoring that does not require tissue penetration. Thus, repositioning of the anchor during a procedure may be easier and less traumatic to the tissue. Anchor 580 advantageously lacks sharp features, e.g., distal tips, which reduces the possibility of puncturing an artery with the anchor. Further, anchor 580 can be anchored to any tissue of sufficient thickness to hold the vacuum. Nonetheless, for some applications, cup 582 (e.g., flange 583 thereof) can have barbs on its tissue-facing side, to augment its suction-cup-based anchoring.

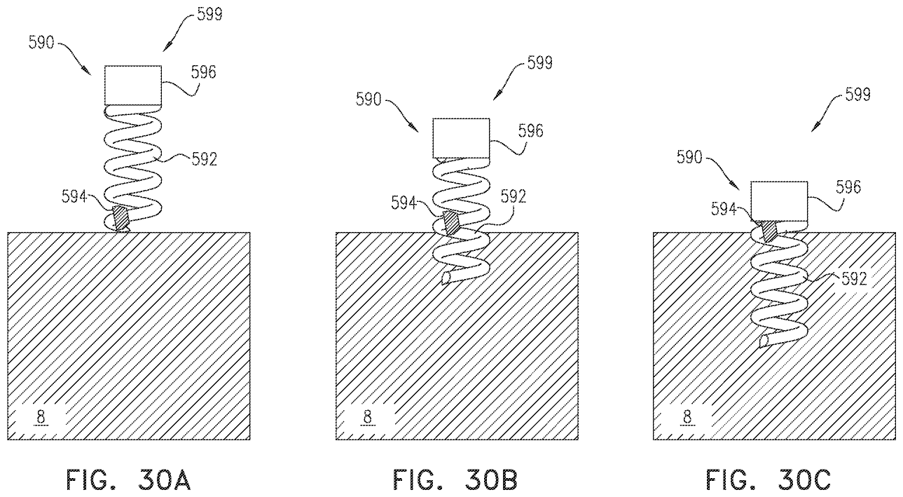

Reference is made to FIGS. 30A-C, which are schematic illustrations of a system 599 for verifying a placement of a tissue anchor in a tissue comprising a tissue anchor 590, in accordance with some applications.

For some applications, and as shown, anchor 590 comprises a head 596 and a tissue-engaging element 592, e.g., a helical tissue-engaging element, having a sharpened distal tip. Tissue-engaging element 592 can be defined by a wire (or a rod or tube) that is shaped to define the tissue-engaging element. For example, for applications in which tissue-engaging element 592 is helical, the wire is curved into a helix. Anchor 590 further comprises a ring 594, slidably coupled to the helical turns of tissue-engaging element 592—e.g., threaded onto the wire that defines the tissue-engaging element. Ring 594 can be radiopaque and/or echogenic, and thus visible via imaging techniques such as fluoroscopy and/or ultrasound.

System 599 can further comprise an anchor driver (not shown), which can share features with any of the anchor drivers described herein, mutatis mutandis.

Prior to deployment of anchor 590, ring 594 can be positioned near the distal tip of tissue-engaging element 592. Ring 594 is shaped and/or dimensioned such that it remains outside of tissue 8, i.e., on the surface thereof. For example, ring 594 can bulge outwardly from the wire that defines the tissue-engaging element, and/or can present a flat face that abuts the tissue surface. As the driver begins to advance tissue-engaging element 592 into a tissue 8, ring 594 responsively slides along tissue-engaging element 592 toward head 596 e.g., while remaining stationary with respect to the surface of the tissue. As the driver continues to screw tissue-engaging element 592 into tissue 8, ring 594 progresses proximally toward head 596 (FIG. 30B). That is, head 596 approaches tissue 8 and ring 594 while the ring remains stationary with respect to the surface of the tissue. When ring 594 reaches head 596, it can become sandwiched between head 596 and the proximal end of tissue-engaging element 592, thus providing an indication that anchor 590 has been completely threaded into tissue 8. More generally, the position of ring 594 with respect to tissue-engaging element 592 and/or head 596 can provide a fluoroscopic indication of the depth to which the tissue-engaging element has been driven into the tissue.

The fit of ring 594 on the wire of tissue-engaging element 592 can be such that the ring is prevented from falling off of the distal end of the tissue-engaging element and/or from prematurely sliding proximally along the tissue-engaging element—e.g., until being pushed by the tissue. However, the fit is also typically such that tissue-engaging element 592 can easily slide through ring 594 as it penetrates the tissue. For example, the fit can be optimized such that friction between tissue-engaging element 592 and ring 594 provides such behavior. For some applications, the outer diameter of the ring can be maximized reduce the risk of tissue penetration. The distal end of the helix can comprise a small step or ridge to prevent ring 594 from sliding off the distal tip. For some applications, ring 594 can include a detent or other catching feature to add holding force to the starting location, and thereby reduce the risk of the ring sliding up prematurely. For some applications, ring 594 can be slanted in a specific direction to more closely match the anticipated angle of anchor 590 relative to the surface of tissue 8.

It is to be noted that, although in the example shown ring 594 is initially disposed near to the distal end of the tissue-engaging element, for some applications the ring can be initially disposed partway (e.g., ⅓ of the way, midway, or ⅔ of the way) toward the head of the anchor—e.g., the ring may only begin to move with respect to the tissue-engaging element after part (e.g., ⅓, ½, or ⅔) of the tissue-engaging element has penetrated into the tissue.

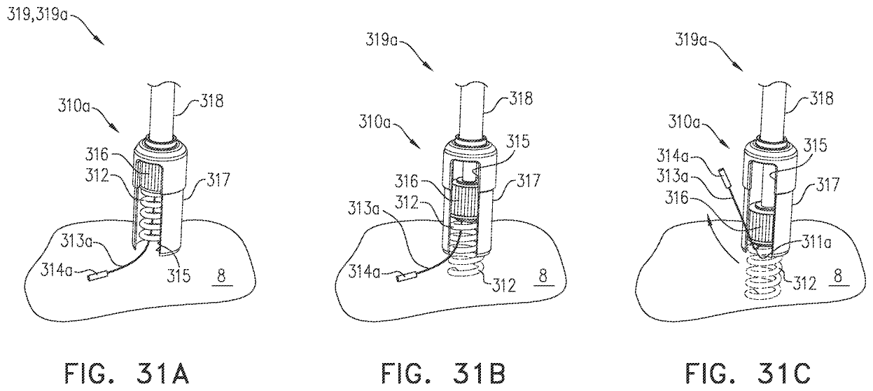

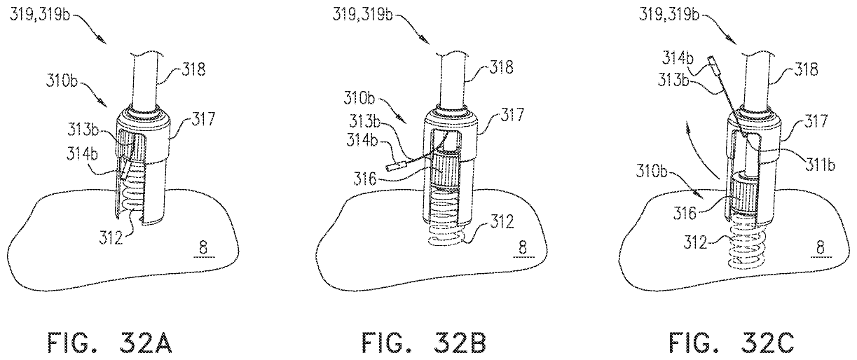

Reference is made to FIGS. 31A-C and 32A-C, which are schematic illustrations of variants of a system 319 that comprises a tissue anchor and an indicator wire, in accordance with some applications.

FIGS. 31A-C show a system 319a, which is a variant of system 319, comprising a tissue anchor 310a that comprises an indicator wire 313a. System 319a typically also comprises a delivery tool that can comprise a driver 318 and/or a delivery capsule 317.

FIGS. 32A-C show a system 319b, which is a variant of system 319 comprising a tissue anchor 310b. System 319b also comprises an indicator wire 313b, which can be a component of a delivery tool of the system. This delivery tool can further comprise driver 318 and/or delivery capsule 317.

For both system 319a and system 319b, the delivery tool can have an extracorporeal proximal portion (not shown), and a distal portion at which delivery capsule 317 is disposed, and which is configured to be advanced into an organ, e.g., vasculature and/or a heart, of a subject. Delivery capsule 317 can be configured to house anchor 310 prior to delivery. For some applications, and as shown, delivery capsule 317 has a longitudinal slit in a lateral wall.

Each of anchors 310a and 310b comprises a head 316 and a tissue-engaging element 312, e.g., a helical tissue-engaging element, extending distally away from the head.

Each of indicator wires 313a and 313b can have a radiopaque tip (314a and 314b, respectively). Furthermore, each of the indicator wires can have a discrete bending site or location (311a and 311b, respectively) at which the indicator wire is biased to form an acute bend. For some applications, and as shown (FIGS. 31C, 32C), the acute bend is located in a proximal region of wire 313. The indicator wire can comprise a heat-set or memory shape material, such that the wire retains a memory of the acute bend even when straightened.

In system 319a, indicator wire 313a can be connected to head 316. Prior to delivery, indicator wire 313a can be partially constrained longitudinally (e.g., straightened) by tissue-engaging element 312—e.g., by extending distally from head 316, caged within the tissue-engaging element (FIG. 31A). During delivery of anchor 310a into a tissue 8, driver 318 advances anchor 310 out of delivery capsule 317, and screws tissue-engaging element 312 into tissue 8. As driver 318 advances tissue-engaging element 312 into tissue 8, indicator wire 313a does not follow the tissue-engaging element, but rather migrates laterally outward along progressively proximal turns of helical tissue-engaging element 312 (FIG. 31B).

At a certain point of the delivery process (e.g., when tissue-engaging element 312 reaches complete insertion into tissue 8, or after a predetermined amount of the wire migrates laterally outward) bending location 311a of indicator wire 313a exits a proximal turn of helical tissue-engaging element 312, allowing the indicator wire to return to define the acute bend at the bending location. This causes tip 314a to deflect abruptly in a proximal direction, such that the tip becomes disposed proximally from the bending location (FIG. 31C).

For some applications, tip 314a can reach proximally as far as, or beyond, head 316. The abrupt change in position of tip 314a is a reflection of sudden release of stress in the straightened indicator wire 313, similar to a spring being released. Observation (e.g., via fluoroscopy) of this abrupt change in position of tip 314a is indicative of successful (e.g., complete) anchoring. Indicator wire 313a can then remain in the tissue connected to anchor 310.

In system 319b, indicator wire 313b can be connected to a component of the delivery tool, e.g., to delivery capsule 317 or to driver 318. Prior to delivery, indicator wire 313b can be partially constrained longitudinally (e.g., straightened) by a component of the delivery tool, such as delivery capsule 317 (FIG. 32A). During delivery of anchor 310b into a tissue 8, driver 318 advances anchor 310b out of delivery capsule 317, and screws tissue-engaging element 312 into tissue 8.

As driver 318 advances tissue-engaging element 312 into tissue 8, indicator wire 313b progressively exits the delivery tool (e.g., delivery capsule 317), e.g., through the longitudinal slit, such that tip 314b moves laterally away from anchor 310b (FIG. 32B). At a certain point of the delivery process, (e.g., when tissue-engaging element 312 reaches complete insertion into tissue 8, or when the wire moves laterally away from the anchor by a predefined amount) bending location 311b of indicator wire 313b exits delivery capsule 317, allowing the indicator wire to return to define the acute bend at the bending location. This causes tip 314b to deflect abruptly in a proximal direction, such that the tip becomes disposed proximally from the bending location (FIG. 32C). For some applications, tip 314b can reach proximally beyond head 316, and/or proximally beyond capsule 317.

For some applications, the wire is configured such that the predefined amount corresponds to the bending location reaching a slit in a lateral wall of the delivery tool. In some other applications, the wire is configured such that the predefined amount corresponds to the bending location reaching a distal rim of the delivery tool. The abrupt change in position of tip 314b is a reflection of sudden release of stress in the straightened indicator wire 313, similar to a spring being released. Observation (e.g., via fluoroscopy) of this sudden change in position of tip 314b is indicative of successful (e.g., complete) anchoring. Indicator wire 313b can then be retracted with the delivery tool.

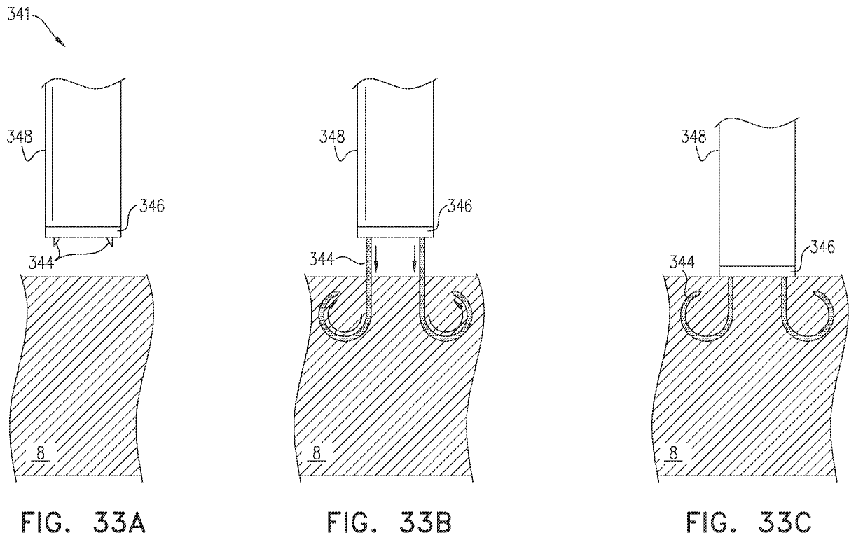

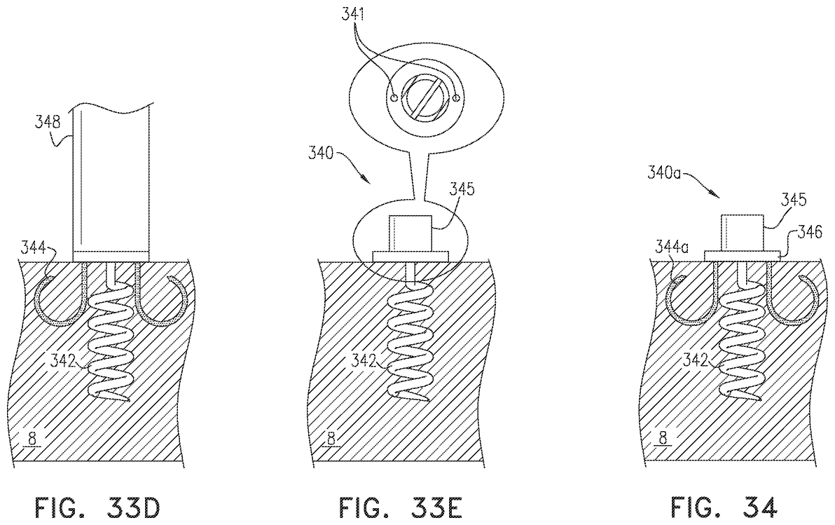

Reference is made to FIGS. 33A-34, which are schematic illustrations of a system 341, and a variant thereof, having multiple tissue-anchoring elements, in accordance with some applications. FIGS. 33A-E show steps in the use of system 341 for anchoring an anchor 340.

System 341 comprises a tissue anchor 340, one or more prongs 344, and a driver 348 for transluminally advancing and anchoring the respective anchor and the prongs. Anchor 340 comprises a head element 345 and a helical tissue-engaging element 342 extending away from the head element in a manner that defines a longitudinal axis of the anchor. Helical tissue-engaging element 342 is fixedly coupled to head element 345.

For some applications, and as shown, prongs 344 are slidably coupled to head element 346. Head element 346 can define openings or holes 343, disposed to match the circumferential position of prongs 344, and through which prongs 344 can be inserted. Head element 345 typically has a smaller diameter than head element 346, and can be rotationally coupled thereto, e.g., by threads or another mechanism that enables the two head elements 345, 346 to be stably coupled and/or decoupled.

For some applications, head element 346 is shaped as a ring, such that tissue-engaging element 342 is insertable therethrough. For some such applications, an outer diameter of head element 345 is greater than an inner diameter of the ring-shaped head element 346.

Prongs 344 are axially translatable and configured to spear the tissue. The prongs can comprise a shape-memory material, e.g., nitinol. As shown, driver 348 can be configured to constrain the prongs (FIG. 33A). The driver is configured to drive prongs 344 into the tissue, with the prongs responsively assuming their memory shape (FIGS. 33B). Prongs 344 can be used to facilitate placement of driver 348 and/or anchor 340 against tissue 8, e.g., by providing a reference force (FIG. 33C).

Driver 348, reversibly engaged with head element 345, can subsequently drive tissue-engaging element 342 into tissue 8 (FIG. 33D). For example, driver 348 can rotate head element 345. While driving tissue-engaging element 342 into tissue 8, driver 348 can pull proximally on prongs 344. By holding the tissue securely against the driver as the driver drives anchor 340 into the tissue, prongs 344 provide a counterforce that minimizes a potential gap between a surface of tissue 8 and a distal surface of the anchor head element 345. With the tissue held in place, the torque-able anchor is rotated into the tissue which provides the primary retention.

For some applications, prongs 344 can then be removed along with driver 348 upon anchoring of tissue-engaging element 342 into tissue 8 (FIG. 33E). This may or may not include retracting prongs 344 via holes 343. Thus, for some applications, prongs 344 may be considered not to be part of anchor 340, e.g., they may be considered to be part of driver 348.

FIG. 34 shows an anchor 340a, which may be considered to be a variant of anchor 340. In contrast to what is shown in FIG. 33E, for some applications, anchor 340a comprises prongs 344a, which may be left in the tissue (e.g., alongside tissue-engaging element 342) upon withdrawal of driver 348. For some applications, anchor 340a is provided with prongs 344a fixed to the head of the anchor. For some applications, prongs 344a begin as slidable with respect to the head of the anchor, and are fixed to the head of the anchor during use. Thus, for some applications, prongs 344a may be considered to be components of anchor 340a.

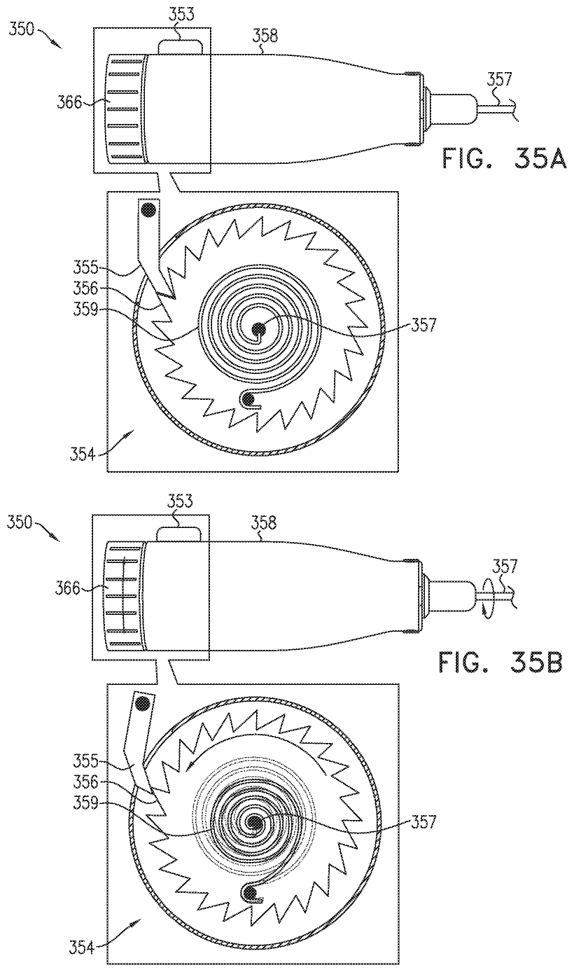

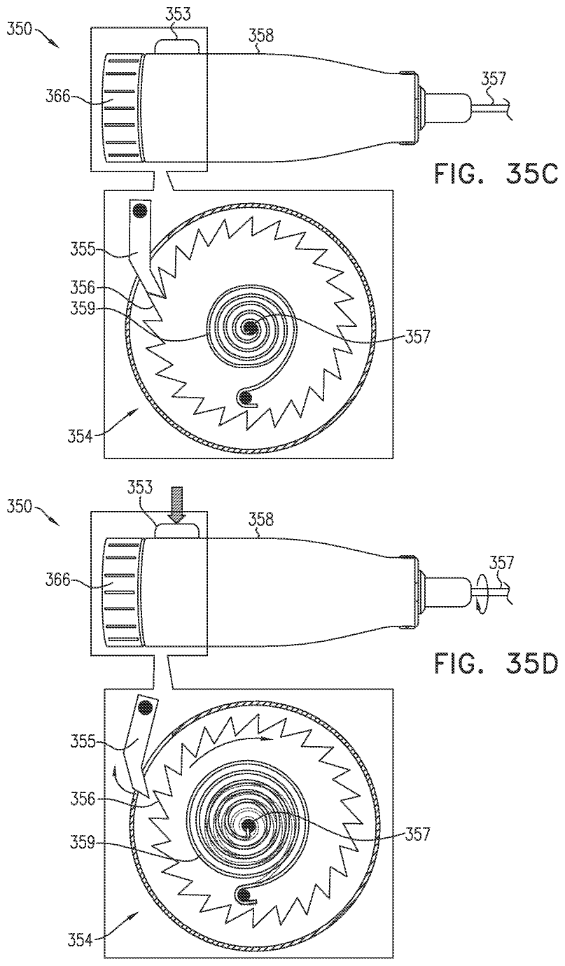

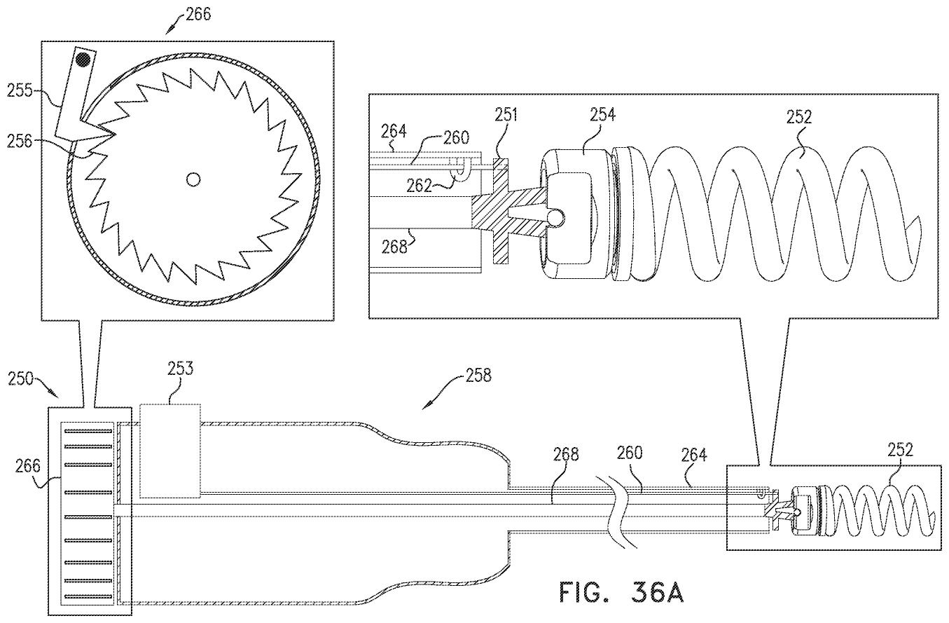

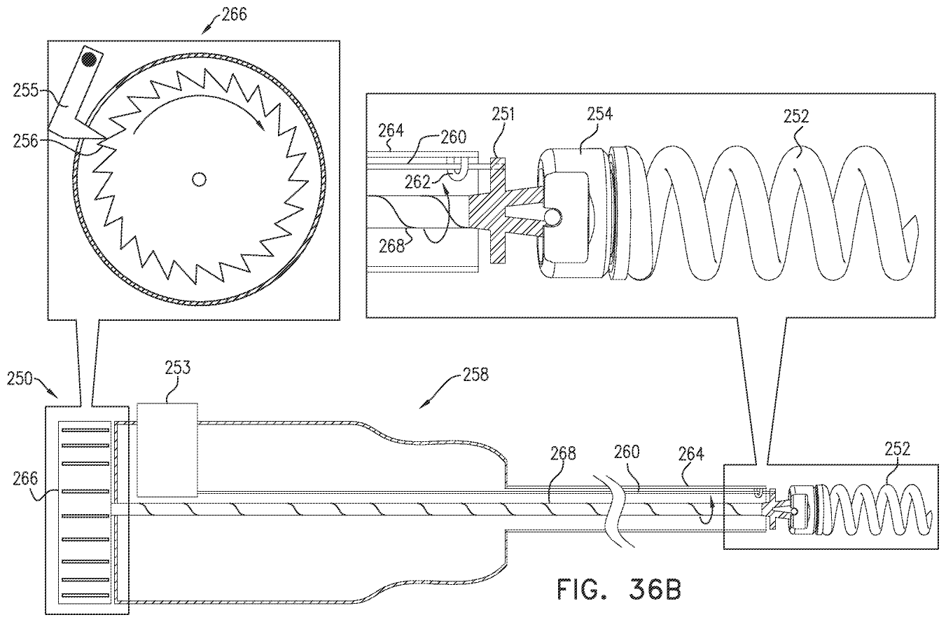

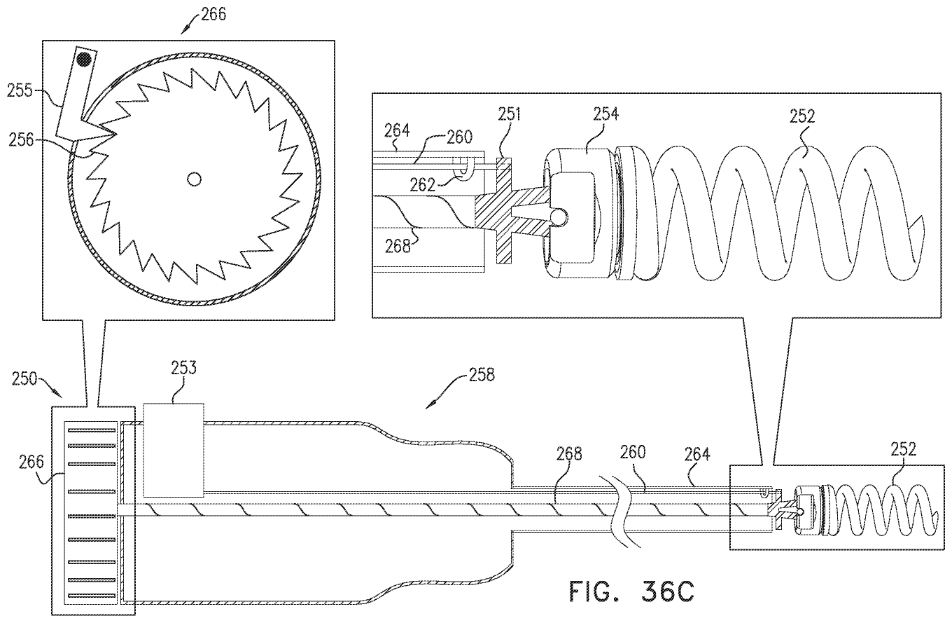

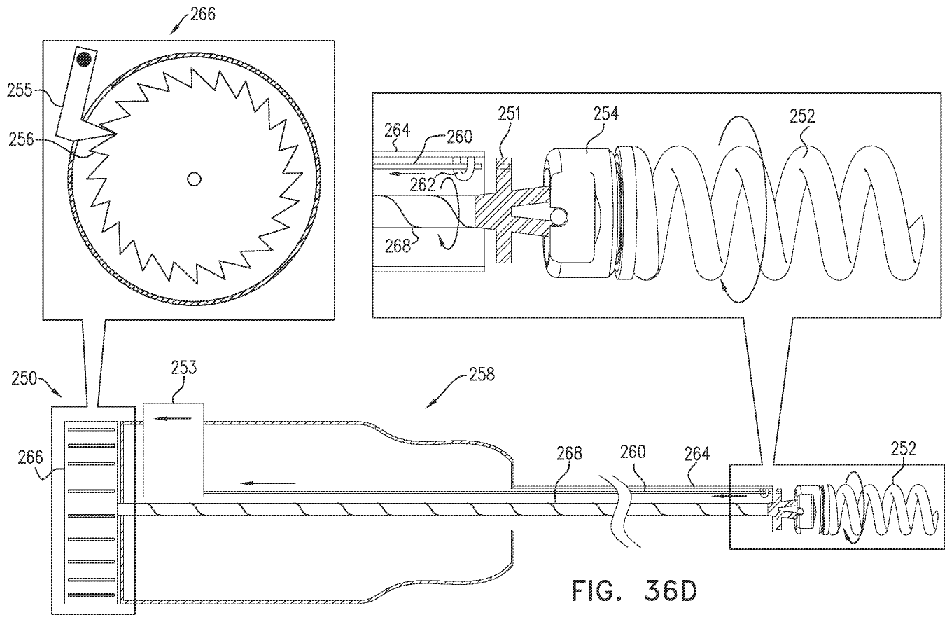

Reference is made to FIGS. 35A-D and 36A-D, which are schematic illustrations of a system 350 and a system 250 for anchoring an anchor to tissue, in accordance with some applications. Each of these systems comprises a driver that includes a drive-head at a distal end of the driver. The drive-head can be configured to be transluminally advanced to the tissue while engaged with the anchor, e.g., to transluminally advance the anchor to the tissue. The system further comprises a torsion spring. The torsion spring can be a discrete component. For some applications, a single component can function as both a driveshaft and as the torsion spring. A winder at a proximal portion of the driver can be operatively coupled to the torsion spring such that operation of the winder winds up the spring. A detent, configured to maintain the spring wound-up, can be disposed at either the proximal end or distal end of the driver. A release interface at a proximal end of the driver can be operatively coupled to the detent such that actuation of the release interface triggers the torsion spring to unwind in a manner that rotates the drive-head.

This solution works by pre-loading the system with potential energy. Upon release of the spring via the detent, the stored potential energy is converted into rotational motion of the driveshaft, which leads to driving of the anchor into the tissue. The system can be automatically actuated, thus advantageously deploying the anchor into the tissue with a single user action, rather than requiring repetitive rotation of a knob controlled by a human operator.

In an exemplary application, a single user action may be performed to store potential energy within the system. A second, simple user action may then release the spring to cause a “self-driving” or automated driving of the anchor. The pre-loading (e.g., winding) may be performed prior to navigating the anchor to the target tissue, or while the anchor is near or at the target tissue.

For some applications, the driver is provided pre-loaded with the potential energy (e.g., pre-wound). For example, the driver may be a single-use driver.

By actuating the release interface at a selected moment of the procedure, stored potential energy is converted into kinetic energy, enabling the torsion spring to spin to its resting state. Although not shown, the driver can include a gear system (e.g., a set of gears) that configures the speed and/or power of the anchor-driving torque.

As shown in FIGS. 35A-D, system 350 comprises a driver 358 that comprises a driveshaft 357 having a distal end that is configured to engage an anchor. The distal end of driveshaft 357 can be transluminally advanced to the tissue while engaged with an anchor.

FIG. 35A shows driver 358 in a resting state. Winder 354 is operatively coupled to (or comprises) a torsion spring 359, such that operation of the winder winds up the spring (FIG. 35B). This is represented by FIG. 35B showing spring 359 contracting medially. Torsion spring 359 is shown as a spiral torsion spring, but it is to be understood that other forms of torsion spring can be alternatively or additionally used.

Winder 354 can comprise a ratchet gear 356 and/or a pawl 355 that maintain spring 359 wound up (FIG. 35C). Both spring 359 and winder 354 can be disposed at a proximal portion of driver 358. In the example shown, winder 354 (e.g., ratchet gear 356 thereof) is fixedly coupled to driveshaft 357. However, it can be coupled via a mechanical linkage, one or more gears, and/or another similar operative coupling.

A release interface 353, e.g., at a proximal end of driver 358, can be operatively coupled to winder 354 such that actuation of release interface 353 releases torsion spring 359 to unwind in a manner that rotates the distal end of the driveshaft (FIG. 35D), thereby driving the anchor into the tissue (not shown). In the example shown, release interface 353 releases torsion spring 359 by disengaging pawl 355 from ratchet gear 356, which, due to spring 359, responsively rotates, driving driveshaft 357. Therefore, for some applications, pawl 355 serves as the detent of driver 358.

As shown in FIGS. 36A-D, system 250 comprises a driver 258 that comprises a driveshaft 268 coupled to a drive-head 251 at a distal end of the delivery tool. Driver 258 can further comprise a sleeve 264, within which driveshaft 268 can be disposed. During transluminal advancement to the tissue, drive-head 251 is engaged with the anchor, e.g., an anchor head 254 thereof. Anchor head 254 can be coupled to a tissue-engaging element 252 that extends distally away from the anchor head.

Driveshaft 268 further serves as the torsion spring of driver 258. For example, driveshaft 268 can function as a torsion bar or a torsion fiber. Similarly to torsion spring 359 of driver 358, driveshaft 268, serving as a torsion spring, is wound up using a winder 266 of driver 258.

FIG. 36A shows driver 258 in a resting state. Winder 266 can be disposed at a proximal end of driver 258, and operatively coupled to driveshaft 268. As noted above, driveshaft 268 can serve as a torsion spring, in a manner that operation of winder 266 winds up the spring, e.g., rotates and twists the driveshaft (FIG. 36B). This is represented in FIG. 36B by oblique lines on driveshaft 268. Similarly to winder 366, winder 266 can comprise a pawl 255 and ratchet 256 that operate to store the potential energy by preventing rotation or spinning in a direction opposite to the one used to wind up the spring. However, as described in more detail hereinbelow, pawl 255 does not serve as the detent that is actuated to trigger the torsion spring to unwind in the manner that rotates the drive-head. Rather, driver 258 comprises a separate detent 260.

Detent 260 can be disposed at a distal end of driver 258, where it engages drive-head 251 in a manner that inhibits rotation of the drive-head. For example, detent 260 can protrude into an opening in drive-head 251. Thus, detent 260 maintains the torsion spring, e.g., driveshaft 268, in a wound-up state (FIG. 36C). This can be facilitated by detent 260 being also engaged with sleeve 264 and/or extending through an eyelet 262, e.g., at or near to the distal end of driver 258. For example, eyelet 262 can be coupled to and/or defined by sleeve 264.

A release interface 253 at a proximal end of the delivery tool can be operatively coupled to detent 260 e.g., via a wire. In the example shown, detent 260 is simply a distal end of such a wire. Via this operative coupling, actuation of release interface 253 disengages detent 260 from drive-head 251. This disengagement triggers driveshaft 268 to unwind in a manner that rotates drive-head 251, deploying tissue-engaging element 252 into the tissue (FIG. 36D).

Reference is again made to FIGS. 35A-D and 36A-D in order to compare system 350 with system 250. In system 350, winder 354 comprises ratchet gear 356 and torsion spring 359. Driver 358 comprises a pawl 355 that serves as the detent at a proximal portion of the driver. Release interface 353 is configured to disengage pawl 355 from ratchet gear 356, thus enabling rotation of spring 359. In contrast, in system 250, winder 266 comprises a pawl 255 and a ratchet gear 256, and driveshaft 268 serves as the torsion spring, with detent 260 being a discrete component, and performing its function at a distal portion of the driver. Furthermore, although both driver 358 and driver 258 are wound up at their respective proximal regions, whereas the stored energy in driver 358 is stored and released at the proximal portion of the driver, the stored energy 258 is released at the distal portion of the driver, and can be stored along the entire length of the driveshaft.

Reference is again made to FIGS. 35A-D and 36A-D. For some applications, the amount of potential energy or tension stored (e.g., storable) in the torsion spring can be calibrated for a specific tissue and/or a specific type of tissue anchor, e.g., to ensure full penetration into the tissue without over-tightening. For example, the driver can be configured to limit the extent to which the torsion spring can be wound up, e.g., by including a slip feature.

Reference is again made to FIGS. 1A-36D. Throughout the present application, (including the specification and the claims), the term “echogenic” should not be construed as being strictly limited to reflection of ultrasonic waves. Rather, it is intended to include applications in which a material or component is effective at reflecting various types of sound and/or electromagnetic waves, such as the X-rays used in fluoroscopy.

Reference is again made to FIGS. 1A-36D. Throughout the present application, (including the specification and the claims), the term “radiopaque” should not be construed as being strictly limited to complete absorption of electromagnetic waves. Rather, it is intended to include applications in which a material or component has enhanced ability to absorb electromagnetic waves (e.g., is radiopaque).

It is to be noted that each of the systems, anchors, and drivers described herein, and/or the other tools or components herein, may be used, mutatis mutandis, as components of systems and/or implants described in one or more of the following references, each of which is incorporated herein by reference: International Patent Application PCT/IL2011/000446 to Miller et al., filed Jun. 6, 2011, which published as WO 2011/154942, International Patent Application PCT/IL2013/050860 to Sheps et al., filed Oct. 23, 2013, which published as WO 2014/064694, International Patent Application PCT/IB2020/060444 to Kasher et al., filed Oct. 27, 2020, which published as WO 2021/084407, International Patent Application PCT/US2021/039587 to Chau et al., filed Jun. 29, 2021, which published as WO 2022/006087, International Patent Application PCT/IB2021/058665 to Halabi et al., filed Sep. 23, 2021, which published as WO 2022/064401, International Patent Application PCT/IB2021/060436 to Tennenbaum et al., filed Nov. 11, 2021, which published as WO 2022/101817, International Patent Application PCT/IB2022/051099 to Tennenbaum et al., filed Feb. 8, 2022, which published as WO 2022/172149, and International Patent Application PCT/US2013/041359 to Rowe et al., filed May 16, 2013, which published as WO 2013/173587.

Similarly, each of the anchors and/or tools described in one or more of these references, may be modified, mutatis mutandis, to include components and/or features of one or more of the anchors and/or tools described herein.

The various systems, devices, apparatuses, etc. in this disclosure can be sterilized (e.g., with heat, radiation, ethylene oxide, hydrogen peroxide, etc.) to ensure they are safe for use with patients, and the methods herein can comprise such sterilization of the associated system, device, apparatus, etc. Furthermore, the scope of the present disclosure includes, for some applications, sterilizing one or more of any of the various systems, devices, apparatuses, etc. in this disclosure.

Any of the techniques, methods, operations, steps, etc. described or suggested herein can be performed on a living animal (e.g., human, other mammal, etc.) or on a non-living simulation, such as a cadaver, a cadaver heart, an anthropomorphic ghost, and/or a simulator device (which may include computerized and/or physical representations of body parts, tissue, etc.).

Although the operations of some of the disclosed methods are described in a particular, sequential order for convenient presentation, it should be understood that this manner of description encompasses rearrangement, unless a particular ordering is required by specific language set forth below. For example, operations described sequentially can in some cases be rearranged or performed concurrently. Moreover, for the sake of simplicity, the attached figures may not show the various ways in which the disclosed systems, apparatuses, devices, methods, etc. can be used in conjunction with other systems, apparatuses, devices, methods, etc.

Example Applications (some non-limiting examples of the concepts herein are recited below):

Example 1. A system for anchoring in a target tissue, the system comprising: (A) an anchor comprising: (i) an inner helical tissue-engaging element; (ii) an outer helical tissue-engaging element; and (iii) a head assembly, comprising: (1) a first head, fixed to a proximal end of the inner tissue-engaging element, and (2) a second head, fixed to a proximal end of the outer tissue-engaging element; and (B) a driver, reversibly interfacing with and engaging the first head and the second head, in order to anchor the anchor in the target tissue.

Example 2. The system according to example 1, wherein a strength of the anchoring is greater than an anchor having only an outer helical tissue-engaging element or only an inner helical tissue-engaging element.

Example 3. The system according to any one of examples 1-2, wherein an outer diameter of the inner helical tissue-engaging element is less than an inner diameter of the outer helical tissue-engaging element.

Example 4. The system according to any one of examples 1-3, wherein the outer helical tissue-engaging element has a tissue-piercing tip at a distal end.

Example 5. The system according to any one of examples 1-4, wherein the inner helical tissue-engaging element has a tissue-piercing tip at a distal end.

Example 6. The system according to any one of examples 1-5, wherein at least one of the inner helical tissue-engaging elements and the outer tissue-engaging element comprise a radiopaque substance.

Example 7. The system according to any one of examples 1-6, wherein at least one of the inner helical tissue-engaging elements and the outer helical tissue-engaging element comprise a polymeric substance.

Example 8. The system according to any one of examples 1-7, wherein the second head comprises an outer cylinder, and the first head comprises an independently moveable inner core that interfaces with the second head.

Example 9. The system according to any one of examples 1-8, wherein the first head and the second head rotate independently.

Example 10. The system according to any one of examples 1-8, wherein the first head and the second head rotate simultaneously.

Example 11. The system according to any one of examples 1-10, wherein the first head fits within the second head when both helical tissue-engaging elements are completely inserted into the target tissue.

Example 12. The system according to any one of examples 1-11, wherein the first head and the second head form a locking mechanism when both helical tissue-engaging elements are fully inserted into the target tissue.

Example 13. The system according to any one of examples 1-12, wherein the driver is configured to advance the inner helical tissue-engaging element into the target tissue prior to advancing the outer helical tissue-engaging element into the target tissue.

Example 14. The system according to any one of examples 1-12, wherein the driver is configured to advance the outer helical tissue-engaging element into the tissue prior to advancing the inner helical tissue-engaging element into the target tissue.

Example 15. The system according to any one of examples 1-14, wherein the second head comprises a concavity with an opening in a central region of the concavity.

Example 16. The system according to example 15, wherein the first head fits rotatably within the concavity of the second head.

Example 17. The system according to example 15, wherein the inner helical tissue-engaging element traverses the opening in the central region of the concavity.

Example 18. The system according to example 15, wherein the opening comprises a set of threads matching a pitch and a handedness of the inner helical tissue-engaging element.

Example 19. The system according to example 18, wherein turns of the inner helical tissue-engaging element advance into the target tissue by running along the set of threads.

Example 20. The system according to example 15, wherein an outer surface of the second head has a non-circular shape.

Example 21. The system according to example 15, wherein an inner surface of the concavity has a circular shape.

Example 22. The system according to example 15, wherein an outer surface of the first head has a circular shape.

Example 23. The system according to example 15, wherein the first head has a non-cylindrical cavity for inserting the driver.

Example 24. The system according to any one of examples 1-23, wherein the driver comprises a first component and a second component.

Example 25. The system according to example 24, wherein the first component engages the first head, and the second component engages the second head.

Example 26. The system according to example 24, wherein the first component fits within a cavity of the first head, and the second component fits over the second head.

Example 27. The system according to example 24, wherein the first component and the second component are independently activatable.

Example 28. The system according to example 24, wherein the first component and the second component are dependently activatable.

Example 29. The system according to any one of examples 1-28, wherein the inner helical tissue-engaging element has a first handedness and a first pitch, and the outer helical tissue-engaging element has a second handedness and a second pitch.

Example 30. The system according to example 29, wherein the first handedness is the same as the second handedness, and the first pitch is the same as the second pitch.

Example 31. The system according to example 29, wherein the first handedness is opposite the second handedness, and the first pitch is the same as the second pitch.

Example 32. The system according to example 29, wherein the first handedness is the same as the second handedness, and the first pitch is non-identical to the second pitch.

Example 33. The system according to example 29, wherein at least one of the first handedness or the first pitch are non-identical to the respective at least one of the second handedness or the second pitch.

Example 34. The system according to any one of examples 1-33, wherein the first head comprises a runner fixed to the inner helical tissue-engaging element.

Example 35. The system according to example 34, wherein the runner advances along turns of the outer helical tissue-engaging element to screw the inner helical tissue-engaging element into the target tissue.

Example 36. The system according to example 34, wherein the runner rides on turns of the outer helical tissue-engaging element as the driver advances the inner helical tissue-engaging element into the target tissue.

Example 37. The system according to example 34, wherein the runner and the second head form a locking mechanism when both tissue-engaging elements are fully inserted into the target tissue.

Example 38. The system according to example 34, wherein the runner comprises a T-bar.

Example 39. The system according to example 34, wherein the runner comprises a flat disc.

Example 40. The system according to example 1, wherein the system is sterilized.

Example 41. A method for anchoring in a real or simulated target tissue, the method comprising: (A) transluminally advancing, to the target tissue, a driver that is coupled to a first head and a second head of a head assembly of an anchor; (B) while the driver remains coupled to the first head and the second head: (i) driving an inner helical tissue-engaging element into the target tissue by using the driver to rotate the first head, and (ii) driving an outer helical tissue-engaging element into the target tissue by using the driver to rotate the second head; and (C) subsequently, decoupling the driver from the head assembly and withdrawing the driver.

Example 42. The method according to example 41, wherein rotating the first head turns the inner helical tissue-engaging element.

Example 43. The method according to any one of examples 41-42, wherein rotating the second head turns the outer helical tissue-engaging element.

Example 44. The method according to any one of examples 41-43, wherein using the driver to rotate the first head comprises using the driver to rotate the first head independently of rotating the second head.

Example 45. The method according to any one of examples 41-44, wherein

driving the inner helical tissue-engaging element into the target tissue comprises driving the inner helical tissue-engaging element into the target tissue prior to driving the outer helical tissue-engaging element into the target tissue.

Example 46. The method according to any one of examples 41-44, wherein driving the inner helical tissue-engaging element into the target tissue comprises driving the inner helical tissue-engaging element into the target tissue subsequently to driving the outer helical tissue-engaging element into the target tissue.

Example 47. The method according to any one of examples 41-44, wherein the first head and the second head rotate simultaneously.

Example 48. The method according to any one of examples 41-44, wherein the driver first rotates the first head, and subsequently rotates the second head.

Example 49. The method according to any one of examples 41-44, wherein the driver first rotates the second head, and subsequently rotates the first head.

Example 50. A system or an apparatus for anchoring in a target tissue of a subject, the system or apparatus comprising an anchor that comprises: (i) a first head; (ii) a helical tissue-engaging element fixed to the first head and extending, away from the first head, distally and helically around an elongate space that is disposed along a longitudinal axis of the anchor; (iii) a second head, coupled to the first head in a manner in which the second head is rotatable with respect to the first head and axially fixed with respect to first head; and (iv) a set of prongs: (1) fixed to the second head, and extending from the second head distally through the elongate space, and (2) arranged with respect to the helical tissue-engaging element such that rotation of the first head with respect to the second head feeds the prongs laterally outward between progressively proximal turns of the helical tissue-engaging element.

Example 51. The system or the apparatus according to example 50, wherein the prongs have elastic properties.

Example 52. The system or the apparatus according to any one of examples 50-51, wherein the prongs have plastic properties.

Example 53. The system or the apparatus according to any one of examples 50-52, wherein the first head and the second head are coupled by a snap-fit mechanism.

Example 54. The system or the apparatus according to any one of examples 50-53, wherein the first head further comprises a non-circular protrusion configured to reversibly couple to a driver.

Example 55. The system or the apparatus according to example 54, wherein

turning the first head via the driver fitting into the non-circular protrusion advances the helical tissue-engaging element into the target tissue.

Example 56. The system or the apparatus according to any one of examples 50-55, wherein the anchor defines a track and a rider, and the second head is coupled to the first head via the rider being slidably coupled to the track.

Example 57. The system or the apparatus according to example 56, wherein the track is annular.

Example 58. The system or the apparatus according to example 56, wherein the rider is slidably coupled to the track by protruding into the track.

Example 59. The system or the apparatus according to example 56, wherein the rider is a first rider of a set of riders, the set of riders being distributed in a ring formation.

Example 60. The system or the apparatus according to example 56, wherein the track is located on the first head and the rider is located on the second head.

Example 61. The system or the apparatus according to example 56, wherein the track is located on the second head and the rider is located on the first head.

Example 62. The system or the apparatus according to example 50, wherein the system or the apparatus is sterilized.

Example 63. A method for anchoring in a real or simulated target tissue, the method comprising: (i) transluminally advancing, to the target tissue, a driver that is coupled to a first head of a head assembly of an anchor, the first head coupled to a helical tissue-engaging element, and a second head of the head assembly fixed to a set of prongs; and (ii) anchoring the anchor to the tissue by rotating the first head such that: (1) the helical tissue-engaging element becomes screwed into the target tissue; and (2) the prongs progressively extend laterally outward between progressively proximal turns of the helical tissue-engaging element into the target tissue.

Example 64. The method according to example 63, wherein the driver rotates the first head by fitting over a non-circular protrusion on the first head.

Example 65. The method according to any one of examples 63-64, wherein the driver rotates the first head by fitting within a non-circular depression on the first head.

Example 66. The method according to any one of examples 63-65, wherein anchoring the anchor to the target tissue by rotating the first head comprises anchoring the anchor to the target tissue by rotating the first head such that the second head advances toward the target tissue without rotating.

Example 67. The method according to any one of examples 63-66, wherein the prongs progressively extending laterally outward between progressively proximal turns of the helical tissue-engaging element anchors the anchor in the target tissue with a strength of anchoring is greater than a method lacking the prongs progressively extending laterally outward between progressively proximal turns of the helical tissue-engaging element.

Example 68. A system or an apparatus for providing supplementary anchoring in a target tissue, comprising: (i) a primary tissue-engaging element; (ii) a head attached to a proximal end of the primary tissue-engaging element; and (iii) a set of supplementary tissue-engaging elements coupled to the head such that, for each supplementary tissue-engaging element of the set, the supplementary tissue-engaging element is: (1) constrainable in a disengaged orientation, and (2) biased toward deflecting away from the disengaged orientation and toward an engaged orientation in which the supplementary tissue-engaging element is closer to the primary tissue-engaging element than in the disengaged orientation.

Example 69. The system or the apparatus according to example 68, wherein each supplementary tissue-engaging element is coupled to the head via an arm.

Example 70. The system or the apparatus according to example 69, wherein the arm has a double-jointed elbow-type articulation between the supplementary tissue-engaging element and the head.

Example 71. The system or the apparatus according to any one of examples 68-70, wherein the set comprises a pair of supplementary tissue-engaging elements positioned opposite each other around the head.

Example 72. The system or the apparatus according to any one of examples 68-70, wherein the set comprises three supplementary tissue-engaging elements positioned at 120-degree intervals around the head.

Example 73. The system or the apparatus according to any one of examples 68-70, wherein the set comprises four supplementary tissue-engaging elements positioned at 90-degree intervals around the head.

Example 74. The system or the apparatus according to any one of examples 68-73, wherein the supplementary tissue-engaging elements are positioned at equal intervals around the head.

Example 75. The system or the apparatus according to any one of examples 68-74, wherein each supplementary tissue-engaging element has a hook that penetrates a surface of the target tissue when disposed in its engaged orientation.

Example 76. The system or the apparatus according to example 68, wherein the system or the apparatus is sterilized.

Example 77. A method for anchoring in a real or simulated target tissue, comprising: (i) transluminally advancing, to the target tissue, a driver that is engaged with a head of an anchor, the head fixed to a primary tissue-engaging element, and articulatably coupled to a set of supplementary tissue-engaging elements; (ii) driving the primary tissue-engaging element into the target tissue; and (iii) subsequently withdrawing the driver, such that the supplementary tissue-engaging elements responsively move toward the primary tissue-engaging element and hook into a surface of the tissue.

Example 78. The method according to example 77, further comprising pulling proximally on the head such that the supplementary tissue-engaging elements articulate with respect to the head.

Example 79. The method according to example 77, further comprising applying a proximal pulling force to the head such that at least some of the proximal pulling force is distributed to the target tissue via the supplementary tissue-engaging elements.

Example 80. A system or an apparatus for anchoring in a target tissue of a subject, the system or the apparatus comprising an anchor that comprises: (i) a head; (ii) a primary tissue-engaging element, extending distally away from the head, thereby defining a longitudinal axis of the anchor; and (iii) a supplementary tissue-engaging element disposed axially between the head and the primary tissue-engaging element and protruding radially outward.

Example 81. The system or the apparatus according to example 80, wherein the supplementary tissue-engaging element protrudes radially outward beyond the head.

Example 82. The system or the apparatus according to example 80, wherein the supplementary tissue-engaging element comprises a radiopaque material.

Example 83. The system or the apparatus according to example 80, wherein the helical tissue-engaging element defines the longitudinal axis of the anchor by extending helically around the axis.

Example 84. The system or the apparatus according to any one of examples 80-83, wherein the supplementary tissue-engaging element is attached to the head at a junction of the head and the primary tissue-engaging element.

Example 85. The system or the apparatus according to any one of examples 80-84, wherein the anchor defines a gap between the supplementary tissue-engaging element and the primary tissue-engaging element.

Example 86. The system or the apparatus according to any one of examples 80-85, wherein the supplementary tissue-engaging element is freely rotatable with respect to the primary tissue-engaging element.

Example 87. The system or the apparatus according to any one of examples 80-86, wherein the supplementary tissue-engaging element is freely rotatable with respect to the head.

Example 88. The system or the apparatus according to any one of examples 80-87, wherein the supplementary tissue-engaging element comprises a ring with protrusions.

Example 89. The system or the apparatus according to example 88, wherein each of the protrusions is a barb that has a point angled distally.

Example 90. The system or the apparatus according to any one of examples 88, each protrusion is a barb that has a point, wherein upon insertion of the tissue anchor into a target tissue, each point is angled toward the target tissue.

Example 91. The system or the apparatus according to example 88, wherein each of the protrusions is a lobe.

Example 92. The system or the apparatus according to example 91, wherein a distal circumferential surface of each lobe is beveled.

Example 93. The system or the apparatus according to example 91, wherein a proximal circumferential surface of each lobe is beveled.

Example 94. The system or the apparatus according to example 91, wherein each lobe has a circumferential directionality that facilitates anchoring of the anchor in the target tissue.

Example 95. The system or the apparatus according to example 94, wherein the circumferential directionality inhibits extrusion of the anchor.

Example 96. The system or the apparatus according to example 94, wherein: (i) the primary tissue-engaging element is configured to be screwed into the target tissue by rotation in a first rotational direction, and (ii) the circumferential directionality configures the ring to resist rotation within the tissue in a second rotational direction more than in the first rotational direction.

Example 97. The system or the apparatus according to any one of examples 80-90, wherein the supplementary tissue-engaging element comprises a ring having an outer edge, the outer edge foldable within the driver to a diameter not substantially greater than the diameter of the head.

Example 98. The system or the apparatus according to any one of examples 80-90, wherein the supplementary tissue-engaging element comprises a ring having barbs at an outer edge of the ring.

Example 99. The system or the apparatus according to example 98, wherein the barbs are designed to create friction between the supplementary tissue-engaging element and the target tissue.

Example 100. The system or the apparatus according to example 98, wherein the supplementary tissue-engaging element and the head collectively define a clutch.

Example 101. The system or the apparatus according to example 100, wherein the clutch is configured to lock upon the ring being pressed against the head.

Example 102. The system or the apparatus according to example 80, wherein the system or the apparatus is sterilized.

Example 103. A method for anchoring in a real or simulated target tissue, comprising: (i) transluminally advancing, to the target tissue, a driver that is reversibly coupled to a head of an anchor, the head fixed to a primary tissue-engaging element, and a supplementary tissue-engaging element disposed between the head and the primary tissue-engaging element; and (ii) anchoring the tissue anchor into the target tissue, such that the supplementary tissue-engaging element engages a surface of the target tissue lateral to the head.

Example 104. The method according to example 103, wherein anchoring the tissue anchor into the target tissue comprises anchoring the tissue anchor into the target tissue such that the supplementary tissue-engaging element provides lateral stabilization to the anchor.

Example 105. A system for verifying a placement of a tissue anchor in a tissue of a subject, the system comprising: (A) an anchor, comprising: (i) a head attached to the tissue-engaging element, the head defining an internal hollow, and a port opening into the internal hollow, the internal hollow being open at a tissue-facing surface of the head; and (ii) a tissue-engaging element, configured to anchor the anchor to the tissue in a manner that places the tissue-facing surface in contact with a surface of the tissue; and (B) a driver: (i) comprising a shaft that defines a channel, and (ii) reversibly couplable to the head in a manner that: (1) enables the driver to anchor the anchor to the tissue by driving the tissue-engaging element into the tissue; and (2) places the channel in fluid communication with the port.

Example 106. The system according to example 105, wherein the reversible coupling between the driver and the head is configured to engage the driver to position the tissue anchor such that the tissue-facing surface of the head contacts the surface of the tissue.

Example 107. The system according to example 106, further comprising a dispenser configured to dispense a contrast agent via the channel and the port into the hollow.

Example 108. The system according to example 107, wherein the dispenser comprises a pump.

Example 109. The system according to example 107, wherein the anchor is configured such that the contact of the tissue-facing surface with the surface of the tissue retains the contrast agent within the hollow.

Example 110. The system according to example 107, wherein the dispenser comprises a capsule containing the contrast agent.

Example 111. The system according to example 110, wherein the capsule is disposed at a distal portion of the driver.

Example 112. The system according to example 110, wherein the capsule is disposed at an extracorporeal proximal portion of the driver.

Example 113. The system according to example 110, wherein the capsule is frangible, and the dispenser is configured to dispense the contrast agent by breaking the capsule.

Example 114. The system according to example 110, wherein the capsule is compressible, and the dispenser is configured to dispense the contrast agent by compressing the capsule.

Example 115. The system according to example 105, wherein the system is sanitized.

Example 116. A method for verifying a placement of a tissue anchor in a tissue of a subject, comprising: (i) transluminally advancing, to the tissue, a driver engaged with a head of a tissue anchor, the tissue anchor including a tissue-engaging element fixed to the head, the head defining an internal hollow accessible via a port; (ii) via the engagement between the driver and the head, driving the tissue-engaging element into the tissue; and (iii) subsequently, via a channel of the driver that is in fluid communication with the port, dispensing a contrast agent into the internal hollow.

Example 117. The method according to example 116, wherein the internal hollow is open at a tissue-facing surface of the head, and wherein driving the tissue-engaging element into the tissue comprises driving the tissue-engaging element into the tissue such that the tissue-facing surface contacts a surface of the tissue.

Example 118. The method according to any one of examples 116-117, wherein dispensing the contrast agent into the internal hollow comprises dispensing the contrast agent into the internal hollow by piercing a capsule containing the contrast agent, the capsule held in the channel until the contrast agent is dispensed.

Example 119. The method according to any one of examples 116-118, wherein dispensing the contrast agent into the internal hollow comprises dispensing the contrast agent into the internal hollow by compressing a capsule containing the contrast agent, the capsule held in the channel until the contrast agent is dispensed.

Example 120. The method according to any one of examples 116-119, wherein anchoring the tissue-engaging element into the tissue comprises anchoring the tissue anchor into the tissue such that the tissue-facing surface of the head contacts a surface of the tissue.

Example 121. The method according to any one of examples 116-120, further comprising radiographically imaging the anchor and the contrast agent.

Example 122. The method according to example 121, wherein radiographically imaging the anchor and the contrast agent comprises radiographically imaging the anchor and the contrast agent to check for a presence of the contrast agent in the hollow of the head.

Example 123. The method according to example 121, wherein radiographically imaging the anchor and the contrast agent comprises radiographically imaging the anchor and the contrast agent within a predetermined amount of time after dispensing the contrast agent.

Example 124. The method according to example 120, wherein when the tissue-facing surface of the head contacts the surface of the tissue, the contrast agent is retained in the hollow.

Example 125. The method according to any one of examples 116-124, wherein anchoring the tissue-engaging element into the tissue comprises anchoring the tissue anchor into the tissue such that the tissue-facing surface of the head fails to contact a surface of the tissue.

Example 126. The method according to example 125, wherein when the tissue-facing surface of the head fails to contact the surface of the tissue, contrast agent dispensed into the internal hollow through the port diffuses out of the internal hollow essentially as it is being dispensed.

Example 127. A system for anchoring in a target tissue, the system comprising: (i) an anchor comprising: (1) an outer helical tissue-engaging element; (2) an inner helical tissue-engaging element disposed coaxially within the outer helical tissue-engaging element; and (3) a head assembly, fixed to a proximal end of the inner tissue-engaging element, and to a proximal end of the outer tissue-engaging element; and (ii) a driver, reversibly engageable with the head assembly, such that rotation of the head assembly by the driver concurrently rotates both tissue-engaging elements.

Example 128. A system or an apparatus for anchoring to a tissue, the system or the apparatus comprising: (A) an anchor comprising: (i) a helical tissue-engaging element; and (ii) a head, comprising: (1) an interface, and (2) a torque limiter, operatively coupling the interface to the tissue-engaging element; and (B) a driver, reversibly engageable with the interface, and configured, while engaged with the interface, to screw the tissue-engaging element into the tissue by applying torque to the interface, and wherein the torque limiter is configured to transfer the torque to the tissue-engaging element, only up to a predefined threshold torque.

Example 129. The system or the apparatus according to example 128, wherein the torque limiter comprises a slip clutch.

Example 130. The system or the apparatus according to any one of examples 128-129, wherein the torque limiter comprises a shear pin.

Example 131. The system or the apparatus according to any one of examples 128-130, wherein the torque limiter is a ball-detent torque limiter.

Example 132. The system or the apparatus according to any one of examples 128-131, wherein the torque limiter comprises a gear set.

Example 133. The system or the apparatus according to any one of examples 128-132, wherein the torque limiter is disposed axially between the interface and the tissue-engaging element.

Example 134. The system or the apparatus according to any one of examples

128-133, wherein the torque limiter limits the torque transferred to the tissue-engaging element by slipping upon the torque exceeding the predefined threshold torque.

Example 135. The system or the apparatus according to any one of examples 128-134, wherein the torque limiter limits the torque transferred to the tissue-engaging element by operatively uncoupling the interface from the tissue-engaging element upon the torque exceeding the predefined threshold torque.

Example 136. The system or the apparatus according to any one of examples 128-135, wherein: (i) the driver is configured to unscrew the tissue-engaging element from the tissue by applying reverse torque to the interface, and (ii) the torque limiter is configured to transfer the reverse torque to the tissue-engaging element even upon the reverse torque exceeding the predefined threshold torque.

Example 137. The system or the apparatus according to any one of examples 128-136, wherein the torque limiter is a pawl-and-spring torque limiter comprising a drive pawl, a series of notches, and a spring configured to hold the drive pawl against the series of notches.

Example 138. The system or the apparatus according to example 137, wherein the spring is fixed to the interface.

Example 139. The system or the apparatus according to example 134, wherein

the spring is a first spring and a second spring, the first spring oriented to limit torque in a direction of rotation, and the second spring oriented to limit torque in an opposite direction of rotation.

Example 140. The system or the apparatus according to example 138, wherein the head further comprises a base, the base: (i) defining a tissue-facing surface, (ii) fixed with respect to the tissue-engaging element, and (iii) defining the series of notches.

Example 141. The system or the apparatus according to example 140, wherein the spring is disposed inside the base.

Example 142. The system or the apparatus according to example 140, wherein the spring is a spiral spring that defines a spring plane, the series of notches lying on the spring plane.

Example 143. The system or the apparatus according to example 142, wherein the series of notches is arranged to circumscribe the spiral spring.

Example 144. The system or the apparatus according to example 142, wherein the spring plane is parallel to the tissue-facing surface.

Example 145. The system or the apparatus according to example 142, wherein the spiral spring is shaped to define a double spiral having two spiral arms.

Example 146. The system or the apparatus according to example 128, wherein the system or the apparatus is sterilized.

Example 147. A method for anchoring to a real or simulated tissue, the method comprising: (i) transluminally advancing, to the tissue, a driver engaged with an interface of an anchor, the anchor including a helical tissue-engaging element, and a torque limiter operatively coupling the interface to the tissue-engaging element; and (ii) using the driver, screwing the tissue-engaging element into the tissue by applying torque to the interface such that the torque limiter transfers the torque to the tissue-engaging element until the torque exceeds a predefined threshold torque and the torque limiter responsively ceases to transfer the torque to the tissue-engaging element.

Example 148. The method according to example 147, further comprising, subsequently to screwing the tissue-engaging element into the tissue, disengaging the driver from the interface and withdrawing the driver.

Example 149. The method according to any one of examples 147-148, further comprising, responsively to the torque limiter ceasing to transfer the torque to the tissue-engaging element, unscrewing the tissue-engaging element from the tissue by applying reverse torque to the interface using the driver.

Example 150. A system or an apparatus for anchoring to a tissue of a subject, the system or the apparatus comprising an anchor comprising: (i) a head, comprising an interface; (ii) a tissue-engaging element, extending distally away from the head; and (iii) a flexible sleeve extending distally away from the head.

Example 151. The system or the apparatus according to example 150, wherein the sleeve comprises a fabric.

Example 152. The system or the apparatus according to any one of examples 150-151, wherein the sleeve comprises a mesh.

Example 153. The system or the apparatus according to any one of examples 150-152, wherein the sleeve comprises a braid.

Example 154. The system or the apparatus according to any one of examples 150-153, wherein the sleeve comprises a flexible film.

Example 155. The system or the apparatus according to any one of examples 150-154, wherein the sleeve comprises a metal.

Example 156. The system or the apparatus according to any one of examples 150-155, wherein the sleeve comprises a polymer.

Example 157. The system or the apparatus according to any one of examples 150-156, wherein a stiffness of a distal part of the sleeve is greater than a stiffness of a proximal part of the sleeve.

Example 158. The system or the apparatus according to any one of examples 150-157, wherein a stiffness of a proximal part of the sleeve is greater than a stiffness of a distal part of the sleeve.

Example 159. The system or the apparatus according to any one of examples 150-158, wherein the sleeve comprises a porous sheet.

Example 160. The system or the apparatus according to example 159, wherein the porous sheet comprises pores having a pore-size greater than 75 microns.

Example 161. The system or the apparatus according to any one of examples 150-160, further comprising a driver, configured to reversibly engage the interface and to drive the tissue-engaging element into the tissue.

Example 162. The system or the apparatus according to example 161, wherein the driver is configured to drive the tissue-engaging element into the tissue in a manner that axially compresses the sleeve between the head and a surface of the tissue.

Example 163. The system or the apparatus according to example 162, wherein the sleeve is configured to facilitate an amount of axial compression of the sleeve, and to resist compression of the sleeve beyond the amount.

Example 164. The system or the apparatus according to example 163, wherein the tissue-engaging element is configured to be driven sufficiently deep into the tissue that the sleeve becomes axially compressed by the amount.

Example 165. The system or the apparatus according to example 162, wherein the sleeve is configured to spread outward on the surface of the tissue upon being axially compressed between the head and the surface of the tissue.

Example 166. The system or the apparatus according to example 162, wherein the sleeve defines a lumen, the tissue-engaging element extends through the lumen, and the anchor is configured such that, the tissue-engaging element progressively exits a distal end of the lumen upon being progressively driven into the tissue.

Example 167. The system or the apparatus according to example 161, wherein the driver is configured to drive the tissue-engaging element into the tissue in a manner that the sleeve remains extending distally away from the head.

Example 168. The system or the apparatus according to example 167, wherein the tissue-engaging element comprises a threaded shaft having a sharp distal tip.

Example 169. The system or the apparatus according to example 168, further comprising a ring attached to a distal end of the flexible sleeve, and threadedly engaged with the threaded shaft.

Example 170. The system or the apparatus according to example 169, wherein the tissue-engaging element is configured to be driven linearly into the tissue.

Example 171. The system or the apparatus according to example 170, wherein the ring is threadedly engaged with the shaft in a manner that maintains the sleeve extending distally away from the head while the tissue-engaging element is driven linearly into the tissue.

Example 172. The system or the apparatus according to example 169, wherein the driver is configured to rotate the threaded shaft with respect to the ring.

Example 173. The system or the apparatus according to example 172, wherein: (i) the driver is configured to rotate the threaded shaft with respect to the ring in a manner that draws the ring toward the head, and (ii) the sleeve is configured to spread radially outward within the tissue, responsively to the ring being drawn toward the head.

Example 174. The system or the apparatus according to any one of examples 150-173, wherein the tissue-engaging element defines a central longitudinal axis of the anchor, and wherein the sleeve is compressible along the central longitudinal axis.

Example 175. The system or the apparatus according to example 174, wherein the sleeve is compressible such that, as the sleeve becomes compressed, the sleeve spreads radially outward from the central longitudinal axis.

Example 176. The system or the apparatus according to example 174, wherein the tissue-engaging element extends through a lumen of the sleeve.

Example 177. The system or the apparatus according to example 176, wherein the sleeve substantially covers the tissue-engaging element.

Example 178. The system or the apparatus according to any one of examples 150-177, further comprising prongs, coupled to the head, and extending, within a lumen of the sleeve, distally away from the head.

Example 179. The system or the apparatus according to example 178, wherein the prongs are covered by the sleeve.

Example 180. The system or the apparatus according to example 178, wherein the prongs comprise barbs.

Example 181. The system or the apparatus according to example 178, wherein the prongs comprise spikes.

Example 182. The system or the apparatus according to example 178, wherein the prongs comprise metal wires.

Example 183. The system or the apparatus according to example 178, wherein the prongs are disposed laterally from the tissue-engaging element.

Example 184. The system or the apparatus according to example 178, wherein the prongs are dimensioned to become exposed from the sleeve upon the sleeve becoming compressed along the central longitudinal axis.

Example 185. The system or the apparatus according to example 178, wherein the anchor comprises a collar via which the prongs are coupled to the head.

Example 186. The system or the apparatus according to example 185, wherein the collar is disposed axially between the head and the tissue-engaging element.

Example 187. The system or the apparatus according to example 185, wherein the collar is rotatably coupled to the head.

Example 188. The system or the apparatus according to any one of examples 150-187, wherein the sleeve further comprises a flexible tubular sheet, and a flexible wire extending helically along the sheet.

Example 189. The system or the apparatus according to example 188, wherein the sheet comprises a fabric.

Example 190. The system or the apparatus according to example 188, wherein the flexible wire is configured to support the sheet being tubular, but is compressible.

Example 191. The system or the apparatus according to example 188, wherein the flexible wire is woven into the sheet.

Example 192. The system or the apparatus according to example 188, wherein the flexible wire is embedded in the sheet.

Example 193. The system or the apparatus according to example 188, wherein the flexible wire is formed from a metal.

Example 194. The system or the apparatus according to example 188, wherein the flexible wire is formed from a polymer.

Example 195. The system or the apparatus according to example 188, wherein the flexible wire is echogenic.

Example 196. The system or the apparatus according to example 188, wherein the flexible wire is radiopaque.

Example 197. The system or the apparatus according to example 196, wherein the sleeve has a length along which the sleeve extends distally away from the head, and the flexible wire extends helically along the sheet for an entire length of the sleeve.

Example 198. The system or the apparatus according to example 150, wherein the system or the apparatus is sterilized.

Example 199. A method for anchoring to a real or simulated tissue of a subject, the method comprising: (i) transluminally advancing, to the tissue, a driver reversibly coupled to an anchor, the anchor including a head, a compressible sleeve, and a helical tissue-engaging element; (ii) using the driver, screwing the tissue-engaging element into the tissue, such that: (1) the sleeve becomes compressed on a surface of the tissue as the tissue-engaging element advances into the tissue; and (2) when the tissue-engaging element is completely anchored in the tissue, the sleeve is disposed between the head and a surface of the tissue.

Example 200. The method according to example 199, wherein screwing the tissue-engaging element into the tissue comprises screwing the tissue-engaging element into the tissue such that a set of short prongs, coupled to the head, become driven into the tissue.

Example 201. The method according to any one of examples 199-200, wherein the sleeve being compressed on the surface of the tissue further comprises the sleeve spreading outward from a central longitudinal axis of the anchor.

Example 202. The method according to any one of examples 199-201, wherein the sleeve being compressed on the surface of the tissue is enhanced by the sleeve comprising a helical wire.

Example 203. The method according to any one of examples 199-202, wherein the sleeve becoming compressed on the surface of the tissue is enhanced by a stiffness of a proximal part of the sleeve being different than a stiffness of a distal part of the sleeve.

Example 204. A system or an apparatus for anchoring to a tissue of a subject, the system or the apparatus comprising a driver and an anchor, the anchor comprising: (i) a head, (ii) a first helical tissue-engaging element; and (iii) a second helical tissue-engaging element; wherein the first helical tissue-engaging element has a fixed diameter not substantially greater than a diameter of the head; wherein the second helical tissue-engaging element has a pre-deployment diameter not substantially greater than the diameter of the head, and a post-deployment diameter substantially greater than the diameter of the head; and wherein deployment of the anchor results in the second tissue-engaging anchor assuming its post-deployment diameter.

Example 205. The system or the apparatus according to example 204, further comprising a delivery tool, and wherein the driver is configured to drive the anchor out of the delivery tool such that the second helical tissue-engaging element expands radially outward from the first helical tissue-engaging element.

Example 206. The system or the apparatus according to any one of examples 204-205, wherein a strength of the anchoring is greater than a system or apparatus having only a single helical tissue-engaging element.

Example 207. The system or the apparatus according to any one of examples 204-206, wherein the first helical tissue-engaging element comprises stainless steel.

Example 208. The system or the apparatus according to any one of examples 204-207, wherein the first helical tissue-engaging element comprises a shape memory material.

Example 209. The system or the apparatus according to any one of examples 204-208, wherein the second helical tissue-engaging element comprises a shape memory material.

Example 210. The system or the apparatus according to example 209, wherein the shape memory material is nitinol.

Example 211. The system or the apparatus according to any one of examples 204-210, further comprising a removable capsule surrounding and constraining the first helical tissue-engaging element and the second helical tissue-engaging element pre-deployment.

Example 212. The system or the apparatus according to example 211, wherein the removable capsule is coupled to the driver.

Example 213. The system or the apparatus according to example 211, wherein the removable capsule defines internal threads which at least one of the first tissue-engaging element and the second tissue-engaging element traverse during deployment.

Example 214. The system or the apparatus according to example 213, wherein the internal threads are present throughout the length of the removable capsule.

Example 215. The system or the apparatus according to example 204 wherein the system or the apparatus is sterilized.

Example 216. A method for anchoring to a real or simulated tissue, the method comprising: (i) transluminally advancing, to the tissue, a driver, and an anchor comprising a head, a first helical tissue-engaging element, and a second helical tissue-engaging element; and (ii) screwing the anchor into the tissue by using the driver to screw the anchor, such that as the anchor advances into the tissue, the second helical tissue-engaging element assumes a preset memory shape having a diameter greater than the diameter of the first helical tissue-engaging element.

Example 217. The method according to example 216, further comprising, subsequently to screwing the tissue-engaging element into the tissue, disengaging the driver from the anchor and withdrawing the driver.

Example 218. The method according to example 217, wherein: (i) transluminally advancing the driver and the anchor comprises transluminally advancing the driver and the anchor while the anchor is disposed within a removable capsule of the driver, the removable capsule having internal threads, and (ii) using the driver to screw the anchor into the tissue, comprises using the driver to screw the anchor out of the removable capsule along the internal threads and into the tissue.

Example 219. A system or an apparatus for anchoring to a tissue of a subject, the system or the apparatus comprising: (i) an anchor comprising: (1) a tissue-engaging element; and (2) a head, having a deployed width greater than an undeployed width; and (ii) a delivery tool, comprising: (1) a tube, having an internal diameter that is smaller than the deployed width of the head, and (2) a driver, configured to drive the anchor out of the tube such that the head expands toward the deployed width.

Example 220. The system or the apparatus according to example 219, wherein the head comprises a shape memory substance.

Example 221. The system or the apparatus according to any one of examples 219-220, wherein a strength of the anchoring is greater than an anchor having the deployed width of the head less than the inner diameter of the tube.

Example 222. The system or the apparatus according to any one of examples 219-221, wherein the tissue-engaging element has a tissue-piercing tip at a distal end.

Example 223. The system or the apparatus according to any one of examples 219-222, wherein the anchor comprises a continuous wire that is shaped to define both the head and the tissue-engaging element.

Example 224. The system or the apparatus according to any one of examples 219-223, wherein a deployed shape of the head comprises a coil.

Example 225. The system or the apparatus according to example 224, wherein the coil defines a planar spiral.

Example 226. The system or the apparatus according to example 224, wherein deployment of the anchor into the tissue is accompanied by the head expanding toward the deployed shape.

Example 227. The system or the apparatus according to any one of examples 219-226, wherein a deployed width of the tissue-engaging element is greater than the internal diameter of the tube.

Example 228. The system or the apparatus according to example 227, wherein deployment of the anchor into the tissue is accompanied by the tissue-engaging element expanding toward the deployed width.

Example 229. The system or the apparatus according to example 227, wherein the tissue-engaging element comprises a shape memory substance.

Example 230. The system or the apparatus according to example 227, wherein a strength of the anchoring is greater than an anchor having the deployed width of the tissue-engaging element smaller than the inner diameter of the tube.

Example 231. The system or the apparatus according to any one of examples 219-230, wherein the anchor is held in an elongated state.

Example 232. The system or the apparatus according to example 231, wherein a width of the elongated state is less than the inner diameter of the tube.

Example 233. The system or the apparatus according to example 231, wherein a strength of the anchoring is greater than an anchor having a deployed width less than the inner diameter of the tube.

Example 234. The system or the apparatus according to example 231, wherein the elongated state of the anchor holds the anchor under tension.

Example 235. The system or the apparatus according to example 234, wherein deployment of the anchor by the driver releases the tension, enabling the tissue-engaging element to advance into the tissue.

Example 236. The system or the apparatus according to example 234, wherein deployment of the anchor by the driver enables the tissue-engaging element to expand toward the deployed width.

Example 237. The system or the apparatus according to example 234, wherein the anchor is withdrawable by pulling tension.

Example 238. The system or the apparatus according to any one of examples 219-237, wherein the anchor comprises a length of memory shape wire.

Example 239. The system or the apparatus according to example 238, wherein the anchor has a final shape set that mimics a one-piece helical tissue-engaging element and head.

Example 240. The system or the apparatus according to any one of examples 219-239, wherein the head comprises a crossbar configured to be grasped by a discrete retrieval tool.

Example 241. The system or the apparatus according to example 240, wherein the anchor is a component of an implant, and wherein the crossbar is configured to be coupled to another component of the implant.

Example 242. The system or the apparatus according to example 219, wherein the system or the apparatus is sterilized.

Example 243. A method for anchoring to a real or simulated tissue, the method comprising: (i) transluminally advancing, to the tissue, a delivery tool comprising a tube and a driver, and an anchor constrained linearly within the delivery tool; (ii) driving the anchor into the tissue by using the driver to deploy the anchor such that, as the anchor advances into the tissue, the anchor forms a tissue-engaging element within the tissue by assuming a preset memory shape; and (iii) subsequently, withdrawing the tube from the anchor and withdrawing the delivery tool.

Example 244. The method according to example 243, wherein deploying the anchor comprises deploying the anchor such that the anchor assumes a diameter greater than an inner diameter of the tube.

Example 245. The method according to any one of examples 243-244, wherein the preset memory shape has a diameter greater than an inner diameter of the tube, and driving the anchor into the tissue comprises driving the anchor into the tissue such that the tissue-engaging element assumes the preset memory shape that has the diameter greater than the inner diameter of the tube.

Example 246. The method according to any one of examples 243-245, wherein withdrawing the tube from the anchor comprises withdrawing the tube from the anchor such that the anchor forms a head at a surface of the tissue.

Example 247. The method according to any one of examples 243-246, wherein withdrawing the tube from the anchor comprises withdrawing the tube from the anchor such that the anchor forms the head to have a diameter greater than an inner diameter of the tube.

Example 248. The method according to any one of examples 243-247, wherein: (i) the anchor has a head, and (ii) withdrawing the tube from the anchor comprises withdrawing the tube from the anchor such that the head expands to have a diameter greater than an inner diameter of the driver.

Example 249. The method according to any one of examples 243-248, wherein deploying the anchor removes the linear constraint, enabling the tissue-engaging element to advance into the tissue.

Example 250. The method according to any one of examples 243-249, wherein driving the anchor into the tissue releases the linear constraint.

Example 251. The method according to any one of examples 243-250, wherein the anchor assuming the preset memory shape comprises the head expanding toward a deployed shape.

Example 252. The method according to any one of examples 243-251, further comprising checking a position of the anchor, and withdrawing the anchor from the tissue by pulling tension if the position requires adjustment.

Example 253. The method according to example 252, wherein withdrawing the anchor from the tissue by pulling tension is accomplished by pulling on a proximal end of the head, the proximal end comprising a crossbar.

Example 254. A system or an apparatus for anchoring to a tissue of a subject, the system or the apparatus comprising: (A) a delivery tool, comprising: (i) a sheath having an inner helical thread and a set of inner axial grooves, and (ii) a driver; and (B) an anchor, disposed within the sheath, and comprising: (i) a head, comprising an interface; (ii) a helical tissue-engaging element, extending distally away from the head, and having a size, pitch, and handedness complementary to the helical thread; and (iii) a second tissue-engaging element, comprising a set of prongs, and: (1) extending distally away from the head, (2) disposed laterally from the helical tissue-engaging element, (3) sized and positioned complementarily to the axial grooves, and (4) rotatably coupled to the helical tissue-engaging element; wherein the driver is configured to drive the anchor distally out of the sheath by rotating the head such that the helical tissue-engaging element advances helically along the helical thread and each of the prongs advances axially along a respective one of the axial grooves.

Example 255. The system or the apparatus according to example 254, wherein the sheath is configured such that the axial grooves intersect the helical thread.

Example 256. The system or the apparatus according to any one of examples 254-255, wherein the prongs comprise barbs.

Example 257. The system or the apparatus according to any one of examples 254-256, wherein the prongs comprise spikes.

Example 258. The system or the apparatus according to any one of examples 254-257, wherein the prongs comprise metal wires.

Example 259. The system or the apparatus according to any one of examples 254-258, wherein the prongs have a preset memory shape, such that each prong is biased to extend laterally in a shape of a proximally-directed hook when released from the sheath.

Example 260. The system or the apparatus according to any one of examples 254-259, wherein the sheath defines a longitudinal slit extending a length of the sheath.

Example 261. The system or the apparatus according to any one of examples 254-260, wherein the anchor comprises a collar via which the prongs are coupled to the head.

Example 262. The system or the apparatus according to example 261, wherein the collar is disposed axially between the head and the tissue-engaging element.

Example 263. The system or the apparatus according to example 261, wherein the collar is rotatably coupled to the head.

Example 264. The system or the apparatus according to example 261, wherein the collar and the prongs are formed from a single unitary component that is shaped to define the collar and the prongs.

Example 265. The system or the apparatus according to example 261, wherein the collar and the prongs are formed by additive manufacturing.

Example 266. The system or the apparatus according to example 261, wherein the collar and the prongs are formed as separate components and subsequently attached to each other.

Example 267. The system or the apparatus according to example 261, wherein the helical thread inhibits expansion of the second tissue-engaging element.

Example 268. The system or the apparatus according to example 254, wherein the system or the apparatus is sterilized.

Example 269. A method for anchoring to a real or simulated tissue, the method comprising: (i) transluminally advancing, to the tissue, a driver; an anchor comprising a head, a helical tissue-engaging element, and a second tissue-engaging element comprising a set of prongs, and an anchor sheath unsheathing the anchor; and (ii) driving the anchor into the tissue by using the driver to deploy the anchor, such that as the anchor advances into the tissue, the prongs assume a preset memory shape having a diameter greater than a diameter of the anchor sheath.

Example 270. The method according to example 269, further comprising, subsequently to driving the first tissue-engaging element into the tissue, disengaging the driver from the anchor and withdrawing the driver.

Example 271. A system or an apparatus for anchoring to a tissue of a subject, the system or the apparatus comprising: (A) an anchor, having a longitudinal axis, and comprising: (i) a head, defining, on the longitudinal axis, an aperture therethrough; and (ii) a set of tissue-engaging prongs, arranged circumferentially around the longitudinal axis, each prong: (1) defining a pocket, and (2) biased to extend laterally from the longitudinal axis, the anchor having an anchoring state in which the prongs extend laterally from the longitudinal axis; and (B) an insert: (i) comprising a set of fingers, corresponding to the set of tissue-engaging prongs, and (ii) dimensioned to be secured to the anchor by extending through the aperture such that the fingers are disposed within the pockets in a manner that constrains the anchor in a delivery state in which the prongs are substantially parallel with the longitudinal axis.

Example 272. The system or the apparatus according to example 271, wherein the insert is configured to be intracorporeally retracted through the aperture in a manner that withdraws the fingers from the pockets.

Example 273. The system or the apparatus according to any one of examples 271-272, wherein the insert comprises a radiopaque material.

Example 274. The system or the apparatus according to any one of examples 271-273, wherein the anchor comprises a radiopaque material.

Example 275. The system or the apparatus according to any one of examples 271-274, wherein in the anchoring state, the insert is configured to be completely withdrawn from the anchor.

Example 276. The system or the apparatus according to any one of examples 271-275, wherein in the anchoring state, the insert is configured to be partially retained within the anchor, obstructing the aperture.

Example 277. The system or the apparatus according to any one of examples 271-276, wherein the pockets face medially in the delivery state.

Example 278. The system or the apparatus according to any one of examples 271-277, wherein the fingers are parallel with the longitudinal axis.

Example 279. The system or the apparatus according to any one of examples 271-278, wherein the fingers are parallel with each other.

Example 280. The system or the apparatus according to any one of examples 271-279, wherein the set of tissue-engaging prongs comprises a shape memory material.

Example 281. The system or the apparatus according to example 280, wherein the shape memory material is nitinol.

Example 282. The system or the apparatus according to any one of examples 271-281, wherein the insert comprises a shaft having a proximal head and a sharp distal tip.

Example 283. The system or the apparatus according to example 282, wherein in the delivery state, the distal tip is configured to extend beyond the prongs.

Example 284. The system or the apparatus according to example 282, wherein the distal tip is configured to serve as a lance.

Example 285. The system or the apparatus according to example 282, wherein the insert is configured to be partially retracted such that the distal tip is configured to remain within the anchor in the anchoring state.

Example 286. The system or the apparatus according to example 271, wherein the system or the apparatus is sterilized.

Example 287. A method for anchoring to a real or simulated tissue, the method comprising: (A) introducing, into the subject: (i) an anchor, in a delivery state thereof, the anchor including: (1) a head, and (2) a set of prongs coupled to the head, and (ii) an insert, disposed through an aperture defined by the head, and constraining the anchor in the delivery state by fingers of the insert being disposed within corresponding pockets defined by the prongs; and (B) while the anchor remains in the delivery state, driving the set of prongs into the tissue; and (C) subsequently, withdrawing the fingers from the pockets by retracting the insert through the aperture such that the anchor transitions toward an anchoring state in which the tissue-engaging prongs extend laterally from the longitudinal axis.

Example 288. The method according to example 287, wherein retracting the insert through the aperture comprises completely removing the insert from the anchor.

Example 289. The method according to any one of examples 287-288, wherein retracting the insert through the aperture comprises withdrawing the fingers from the pockets such that the insert remains disposed through the aperture.

Example 290. A system or an apparatus for anchoring to a tissue of a subject, the system or the apparatus comprising an anchor that comprises: (i) a head; and (ii) a tissue-engaging element that comprises: (1) a shaft having a lateral wall, and a pointed tip configured to facilitate insertion of the lateral wall into the tissue; and (2) a balloon, inflatable to expand radially away from the lateral wall within the tissue.

Example 291. The system or the apparatus according to example 290, wherein the shaft of the tissue-engaging element is hollow.

Example 292. The system or the apparatus according to any one of examples 290-291, wherein the shaft of the tissue-engaging element comprises a sharp distal tip configured to facilitate entry of the tissue-engaging element into the tissue.

Example 293. The system or the apparatus according to any one of examples 290-292, wherein the balloon occupies substantially a complete inner dimension of the shaft of the tissue-engaging element.

Example 294. The system or the apparatus according to any one of examples 290-293, wherein the shaft comprises a series of windows via which the balloon is configured to expand radially away from the lateral wall.

Example 295. The system or the apparatus according to example 294, wherein the series of windows comprises exactly two windows.

Example 296. The system or the apparatus according to example 294, wherein the series of windows comprises exactly three windows.

Example 297. The system or the apparatus according to example 294, wherein the series of windows comprises exactly four windows.

Example 298. The system or the apparatus according to example 294, wherein the series of windows comprises exactly five windows.

Example 299. The system or the apparatus according to example 294, wherein the series of windows comprises exactly six windows.

Example 300. The system or the apparatus according to any one of examples 290-299, wherein the head defines an aperture that provides fluid communication with the balloon.

Example 301. The system or the apparatus according to example 300, wherein the aperture is fitted with a closure.

Example 302. The system or the apparatus according to example 301, wherein the closure serves as a one-way valve.

Example 303. The system or the apparatus according to example 301, further comprising a balloon expander.

Example 304. The system or the apparatus according to example 303, wherein the balloon expander is in fluid communication with the balloon via the closure.

Example 305. The system or the apparatus according to example 304, wherein the balloon expander contains a fluid.

Example 306. The system or the apparatus according to example 305, wherein the balloon expander is configured to inflate the balloon by injecting the fluid via the closure.

Example 307. The system or the apparatus according to example 306, wherein the fluid is saline.

Example 308. The system or the apparatus according to example 306, wherein the fluid comprises a resin.

Example 309. The system or the apparatus according to example 308, wherein the resin is an epoxy resin.

Example 310. The system or the apparatus according to example 308, wherein the resin is configured to harden by exposure to UV light.

Example 311. The system or the apparatus according to example 308, further comprising an optical fiber, coupled to the balloon expander, and configured to transmit UV light to the balloon.

Example 312. The system or the apparatus according to any one of examples 290-311, further comprising a delivery tool, configured to percutaneously advance the anchor to the tissue.

Example 313. The system or the apparatus according to example 312, wherein the delivery tool comprises an optical fiber configured to transmit UV light to the balloon.

Example 314. The system or the apparatus according to example 312, wherein the delivery tool is configured to insert the tissue-engaging element into the tissue.

Example 315. The system or the apparatus according to example 312, wherein the delivery tool comprises a balloon expander, the delivery tool being couplable to the anchor in a manner in which the balloon expander is in fluid communication with the balloon.

Example 316. The system or the apparatus according to example 315, wherein the balloon expander contains a fluid, and is configured to inflate the balloon by injecting the fluid into the balloon.

Example 317. The system or the apparatus according to example 316, wherein the fluid is saline.

Example 318. The system or the apparatus according to example 306, wherein the fluid comprises a resin.

Example 319. The system or the apparatus according to example 318, wherein the resin is an epoxy resin.

Example 320. The system or the apparatus according to example 318, wherein the resin is configured to harden by exposure to UV light.

Example 321. The system or the apparatus according to example 290, wherein the system or the apparatus is sterilized.

Example 322. A method for anchoring to a real or simulated tissue, the method comprising: (A) transluminally advancing, to the tissue, an anchor that includes: (i) a head, and (ii) a tissue-engaging element that includes a shaft and a balloon; (B) using a driver, driving the tissue-engaging element into the tissue; and (C) subsequently, inflating the balloon within the tissue by injecting a fluid thereinto, such that the balloon expands radially away from the shaft within the tissue.

Example 323. The method according to example 322, wherein injecting the fluid comprises injecting the fluid via a one-way valve in the shaft.

Example 324. The method according to any one of examples 322-323, wherein

driving the tissue-engaging element into the tissue comprises driving the tissue-engaging element distally into the tissue with a sharp distal tip of the shaft penetrating into the tissue.

Example 325. The method according to any one of examples 322-324, wherein inflating the balloon such that the balloon expands radially away from the shaft, comprises inflating the balloon such that the balloon expands radially through a set of windows in the shaft.

Example 326. The method according to any one of examples 322-325, wherein injecting the fluid comprises injecting a polymeric substance.

Example 327. The method according to example 326, further comprising, subsequently to injecting the polymeric substance, hardening the polymeric substance by exposing the polymeric substance to UV light.

Example 328. The method according to example 327, wherein exposing the polymeric substance to UV light comprises transmitting the UV light via an optical fiber included in the driver.

Example 329. A system or an apparatus for anchoring to a tissue of a subject, the system or the apparatus comprising: (A) an anchor, comprising: (i) a cup, defining a cavity, and having a flexible flange that defines a rim of the cup, (ii) a head having a port opening into the cavity; and (B) a delivery tool, configured to: (i) percutaneously advance the anchor to the tissue, (ii) place the flexible flange against the tissue, and (iii) while the flexible flange remains against the tissue, anchor the anchor to the tissue by applying suction to the cup via the port.

Example 330. The system or the apparatus according to example 329, wherein the port comprises a check valve.

Example 331. The system or the apparatus according to any one of examples 329-330, wherein: (i) the tissue is tissue of an annulus of a valve of a heart of the subject, and (ii) wherein the delivery tool is configured to: (1) place the flexible flange against the tissue of the annulus, and (2) while the flexible flange remains against the tissue of the annulus, anchor the anchor to the annulus of the valve by applying suction to the cup via the port.

Example 332. The system or the apparatus according to any one of examples 329-331, wherein the flange is configured to seal against the tissue.

Example 333. The system or the apparatus according to any one of examples 329-332, wherein the flange comprises soft silicone molded over an outside rim of the cup.

Example 334. The system or the apparatus according to any one of examples 329-333, wherein the cup is less flexible than the flange.

Example 335. The system or the apparatus according to any one of examples 329-334, wherein the cup comprises a stiff material.

Example 336. The system or the apparatus according to example 335, wherein the stiff material is a metal.

Example 337. The system or the apparatus according to example 335, wherein the stiff material is a plastic.

Example 338. The system or the apparatus according to any one of examples 329-337, wherein the port comprises a valve.

Example 339. The system or the apparatus according to example 338, wherein the valve comprises a valve member that comprises a stopper that secures the valve member in place.

Example 340. The system or the apparatus according to example 329, wherein the system or the apparatus is sterilized.

Example 341. A method for anchoring to a real or simulated tissue, the method comprising: (i) transluminally advancing, to the tissue, an anchor that includes: (1) a cup defining a cavity and having a flexible flange, and (2) a head having a port that opens into the cavity; and (ii) using a delivery tool, placing the flexible flange against the tissue, and (iii) while the flexible flange remains against the tissue, anchoring the anchor to the tissue by applying suction to the cup via the port.

Example 342. The method according to example 341, wherein applying suction to the cup comprises applying suction to the cup in a manner that seals the flange to the tissue.

Example 343. The method according to any one of examples 341-342, wherein applying suction to the cup comprises sucking fluid out of the cup via the port.

Example 344. The method according to any one of examples 341-343, wherein the tissue is simulated tissue of a heart, and wherein transluminally advancing comprises transluminally advancing to the tissue of the heart.

Example 345. The method according to any one of examples 341-344, wherein the tissue is simulated tissue of an annulus of a valve of the heart, and wherein anchoring the anchor to the tissue comprises anchoring the anchor to the annulus.

Example 346. An anchor, comprising: (i) a head; (ii) a tissue-engaging element having a sharpened distal tip, and configured to be driven into the tissue; and (iii) a ring, slidably coupled to the helical tissue-engaging element, such that, responsively to driving of the helical tissue-engaging element into the tissue, the ring slides along tissue-engaging element toward the head.

Example 347. The anchor according to example 346, wherein the ring comprises a radiopaque material.

Example 348. The anchor according to any one of examples 346-347, wherein the ring comprises an echogenic material.

Example 349. The anchor according to any one of examples 346-348, wherein the ring is configured to resist entering the tissue as the driver advances the tissue-engaging element into the tissue.

Example 350. The anchor according to any one of examples 346-349, wherein the tissue-engaging element is a helical tissue-engaging element configured to be screwed into the tissue.

Example 351. The anchor according to any one of examples 346-350, further comprising a driver configured to advance the helical tissue-engaging element into the tissue.

Example 352. The anchor according to example 351, wherein the ring is configured to slide proximally along progressive turns of the helical tissue-engaging element as the driver advances the tissue-engaging element into the tissue.

Example 353. The anchor according to example 346, wherein the anchor is sterilized.

Example 354. A method for verifying a placement of a tissue anchor in a real or simulated tissue of a subject, the method comprising: (i) transluminally advancing, to the tissue, a driver engaged with a head of an anchor, the anchor further including: (1) a tissue-engaging element, extending distally from the head, and having a sharpened distal tip, and (2) a ring slidably coupled to the tissue-engaging element; (ii) driving the tissue-engaging element into the tissue; and (iii) fluoroscopically viewing the anchor to determine a position of the ring along the tissue-engaging element.

Example 355. The method according to example 354, wherein the tissue-engaging element is a helical tissue-engaging element, and wherein driving the tissue-engaging element into the tissue comprises screwing the helical tissue-engaging element into the tissue.

Example 356. The method according to any one of examples 354-355, further comprising, responsively to determining the position of the ring along the tissue-engaging element, driving the tissue-engaging element further into the tissue.

Example 357. The method according to any one of examples 354-356, further comprising, responsively to determining the position of the ring along the tissue-engaging element, disengaging the driver from the head of the anchor.

Example 358. A system or an apparatus for use with a tissue of a subject, the system or the apparatus comprising: (i) an anchor, comprising: (1) a head; and (2) a helical tissue-engaging element extending distally away from the head; and (ii) an indicator wire, having a tip, and a discrete bending location at which the indicator wire is biased to form an acute bend, and (iii) a delivery tool, comprising a driver that is engaged with the anchor, the delivery tool being coupled to the indicator wire in a manner that: (1) defines a delivery state of the system or the apparatus, in which the delivery tool is configured to advance the anchor to the tissue while the indicator wire is constrained such that the tip is disposed distally from the bending location, (2) configures the delivery tool to progressively drive the tissue-engaging element into the tissue in a manner that progressively feeds the indicator wire out of the delivery tool, the indicator wire being configured such that, in response to the driver achieving a predefined amount of driving of the anchor into the tissue, the indicator wire abruptly bends to form the acute bend at the discrete bending location.

Example 359. The system or the apparatus according to example 358, wherein the indicator wire is configured such that the predefined amount corresponds to the bending location reaching a slit in a lateral wall of the delivery tool.

Example 360. The system or the apparatus according to any one of examples 358-359, wherein the indicator wire is configured such that the predefined amount corresponds to the bending location reaching a distal rim of the delivery tool.

Example 361. The system or the apparatus according to any one of examples 358-360, wherein the indicator wire is configured such that the predefined amount corresponds to the helical tissue-engaging element being screwed fully into the tissue.

Example 362. The system or the apparatus according to any one of examples 358-361, wherein the distal tip is radiopaque.

Example 363. The system or the apparatus according to any one of examples 358-362, wherein the indicator wire is attached to the head.

Example 364. The system or the apparatus according to any one of examples 358-363, wherein the indicator wire is attached to the driver.

Example 365. The system or the apparatus according to any one of examples 358-364, wherein the indicator wire is configured such that, in response to the driver achieving the predefined amount of driving of the anchor into the tissue, the distal tip abruptly springs proximally by the wire forming the acute bend at the discrete bending location.

Example 366. The system or the apparatus according to any one of examples 358-365, wherein the delivery tool further comprises a delivery capsule, the delivery tool configured to percutaneously deliver the anchor to the tissue while the anchor is disposed within the delivery capsule.

Example 367. The system or the apparatus according to example 366, wherein the indicator wire is attached to the delivery capsule.

Example 368. The system or the apparatus according to example 366, wherein the delivery capsule has a lateral wall that defines a longitudinal slit therein.

Example 369. The system or the apparatus according to example 368, wherein the indicator wire is configured to exit the delivery capsule via the longitudinal slit.

Example 370. The system or the apparatus according to example 358, wherein the system or the apparatus is sterilized.

Example 371. A method for verifying a placement of an anchor in a real or simulated tissue, the method comprising: (i) using a delivery tool, transluminally advancing an anchor to the tissue, the anchor including a head and a tissue-engaging element extending distally away from the head; and (ii) using a driver of the delivery tool, engaged with the head of the anchor, progressively driving the tissue-engaging element into the tissue in a manner that progressively feeds an indicator wire out of the delivery tool, such that, upon achieving a predetermined amount of driving, the indicator wire abruptly bends at a predefined discrete bending location of the wire.

Example 372. The method according to example 371, wherein: (i) the indicator wire includes a radiopaque distal tip, and (ii) progressively driving the tissue-engaging element into the tissue comprises progressively driving the tissue-engaging element into the tissue such that, upon achieving the predetermined amount of driving, the distal tip springs proximally.

Example 373. The method according to any one of examples 371-372, wherein the tissue-engaging element is a helical tissue-engaging element, and driving the tissue-engaging element into the tissue comprises screwing the tissue-engaging element into the tissue.

Example 374. The method according to any one of examples 371-373, further comprising fluoroscopically determining a position of the indicator wire.

Example 375. The method according to example 374, further comprising, responsively to determining the position of the indicator wire, driving the tissue-engaging element further into the tissue.

Example 376. The method according to example 374, further comprising, responsively to determining the position of the indicator wire, disengaging the driver from the anchor.

Example 377. The method according to example 371, further comprising, sterilizing the delivery tool or the anchor.

Example 378. A system or an apparatus for use with tissue of a subject, the system or the apparatus comprising: (i) an anchor head; (ii) a tissue-engaging element, fixed to the anchor head; (iii) a prong, having a resting state in which the prong is shaped to define a hook; (iv) a driver, a distal end of which is transluminally advanceable to the tissue while engaged with the anchor head, the driver being configured to drive: (1) the prong into the tissue such that the prong curves to form the hook within the tissue, and (2) the tissue-engaging element into the tissue, facilitated by a counterforce provided via the prong in the tissue.

Example 379. The system or the apparatus according to example 378, wherein the system or the apparatus comprises an anchor, the anchor comprising the anchor head, the tissue-engaging element, and the prong.

Example 380. The system or the apparatus according to any one of examples 378-379, wherein the system or the apparatus comprises a delivery tool, the delivery tool comprising the driver and the prong.

Example 381. The system or the apparatus according to any one of examples 378-380, wherein the prong is one of a set of prongs, and wherein the prongs of the set are distributed around the anchor head.

Example 382. The system or the apparatus according to any one of examples 378-381, wherein the prong is one of a set of prongs, and wherein the set comprises two prongs positioned opposite each other around the anchor head.

Example 383. The system or the apparatus according to any one of examples 378-382, wherein the prong is one of a set of prongs, and wherein the set comprises three prongs positioned at 120-degree intervals around the anchor head.

Example 384. The system or the apparatus according to any one of examples 378-383, wherein the prong is one of a set of prongs, and wherein the set comprises four prongs positioned at 90-degree intervals around the anchor head.

Example 385. The system or the apparatus according to any one of examples 378-384, wherein the system or the apparatus is sterilized.

Example 386. The system or the apparatus according to any one of examples 378-385, wherein the prong comprises a shape-memory alloy.

Example 387. The system or the apparatus according to any one of examples 378-386, wherein the prong comprises a radiopaque material.

Example 388. The system or the apparatus according to example 378-387, wherein the anchor head is configured to limit lateral movement of the prong.

Example 389. The system or the apparatus according to any one of examples 378-388, wherein the anchor head is configured to constrain the prong within the tissue.

Example 390. The system or the apparatus according to any one of examples 378-389, wherein the anchor head defines a hole, and wherein the driver is configured to drive the prong into the tissue through the hole.

Example 391. The system or the apparatus according to example 390, wherein the driver is configured to remove the prong from the tissue by retracting the prong proximally through the hole.

Example 392. A method for anchoring in a real or simulated tissue, comprising: (i) transluminally advancing, to the tissue: (1) a distal end of a driver, and (2) engaged by the distal end of the driver, an anchor head fixed to a tissue-engaging element; (ii) using the driver, driving a prong into the tissue such that the prong curves to form a hook within the tissue; and (iii) using the driver, driving the tissue-engaging element into the tissue, facilitated by a counterforce provided via the prong in the tissue.

Example 393. The method according to example 392, wherein the method further comprises sterilizing the prong.

Example 394. The method according to any one of examples 392-393, wherein the prong comprises a radiopaque material, and wherein the method further comprises imaging the radiopaque prong in the tissue.

Example 395. The method according to any one of examples 392-394, wherein driving the tissue-engaging element into the tissue comprises screwing the tissue-engaging element into the tissue.

Example 396. The method according to any one of examples 392-395, further comprising sterilizing the driver.

Example 397. The method according to any one of examples 392-396, further comprising sterilizing the anchor head fixed to the tissue-engaging element.

Example 398. The method according to any one of examples 392-397, further comprising disengaging the anchor head from the driver, and withdrawing the driver.

Example 399. The method according to any one of examples 392-398, further comprising removing the prong from the tissue while leaving the tissue-engaging element within the tissue.

Example 400. The method according to any one of examples 392-399, further comprising disengaging the anchor head from the driver, and withdrawing the driver, leaving the tissue-engaging element and the prong within the tissue.

Example 401. The method according to any one of examples 392-400, wherein the prong comprises a shape-memory alloy, and wherein driving the prong into the tissue comprises driving the prong into the tissue such that the prong curves to form the hook by the shape-memory alloy assuming a memory shape thereof.

Example 402. The method according to any one of examples 392-401, wherein the prong is a first prong of a set of prongs, and the method comprises driving the set of prongs into the tissue.

Example 403. The method according to any one of examples 392-402, wherein: (i) the counterforce is provided by applying a proximal pulling force to the prong, and (ii) driving the tissue-engaging element into the tissue, facilitated by the counterforce comprises driving the tissue-engaging element into the tissue, facilitated by the counterforce that is provided by the proximal pulling force applied to the prong.

Example 404. The method according to example 403, wherein the prong comprises a set of prongs, and wherein applying the proximal pulling force to the prong comprises applying a proximal pulling force to the set of prongs, such that the prongs engage the tissue.

Example 405. The method according to example 404, wherein applying the proximal pulling force to the set of prongs is applied in a manner that pulls a surface of the tissue toward the driver while driving the tissue-engaging element into the tissue.

Example 406. The method according to example 404, wherein the set of prongs are shape-memory prongs, and wherein driving the supplemental tissue-engaging element into the tissue comprises driving the shape-memory prongs into the tissue, such that the prongs assume their memory shape within the tissue.

Example 407. A system or an apparatus for anchoring an anchor to tissue, the system or the apparatus comprising a driver that comprises: (i) a drive-head at a distal end of the driver, the drive-head configured to be transluminally advanced to the tissue while engaged with the anchor; (ii) a torsion spring; (iii) a winder at a proximal portion of the driver, and operatively coupled to the torsion spring such that operation of the winder winds up the spring; (iv) a detent, configured to maintain the spring wound-up; and (v) a release interface at a proximal end of the driver, operatively coupled to the detent such that actuation of the release interface triggers the spring to unwind in a manner that rotates the drive-head.

Example 408. The system or the apparatus according to example 407, wherein the driver is configured to limit an extent to which the winder can wind up the spring.

Example 409. The system or the apparatus according to any one of examples 407-408, wherein the torsion spring is a torsion bar.

Example 410. The system or the apparatus according to any one of examples 407-409, wherein the torsion spring is a spiral torsion spring.

Example 411. The system or the apparatus according to any one of examples 407-410, wherein the torsion spring is a torsion fiber.

Example 412. The system or the apparatus according to any one of examples 407-411, wherein the drive-head is sterilized.

Example 413. The system or the apparatus according to any one of examples 407-412, wherein the anchor is sterilized.

Example 414. The system or the apparatus according to any one of examples 407-413, wherein the torsion spring is sterilized.

Example 415. The system or the apparatus according to any one of examples 407-414, wherein the winder is sterilized.

Example 416. The system or the apparatus according to any one of examples 407-415, wherein the driver is sterilized.

Example 417. The system or the apparatus according to any one of examples 407-416, wherein the winder comprises a slip-clutch device.

Example 418. The system or the apparatus according to any one of examples 407-417, wherein the winder comprises a pawl and a ratchet gear.

Example 419. The system or the apparatus according to any one of examples 407-418, wherein the winder comprises a ratchet gear, and the driver comprises a pawl that serves as the detent.

Example 420. The system or the apparatus according to example 419, wherein the release interface is operatively coupled to the pawl such that actuation of the release interface triggers the spring to unwind by disengaging the pawl from the ratchet gear.

Example 421. The system or the apparatus according to any one of examples 407-420, wherein: (i) at a distal portion of the driver, the driver has an eyelet, and (ii) the detent comprises a wire that extends through the eyelet to engage the drive-head.

Example 422. The system or the apparatus according to example 421, wherein the wire is operatively coupled to the release interface, such that actuation of the release interface triggers the spring to unwind by disengaging the wire from the drive-head.

Example 423. The system or the apparatus according to example 421, wherein: (i) the driver further comprises a sleeve, extending from the proximal portion of the driver to the distal portion of the driver, the eyelet being fixed to the sleeve; and (ii) the driver comprises a driveshaft that: (1) extends through the sleeve, (2) operatively couples the winder to the drive-head, and (3) serves as the torsion spring.

Example 424. The system or the apparatus according to example 423, wherein the eyelet is disposed inside the sleeve.

Example 425. A method for anchoring to a tissue of a subject, the method comprising: (i) transluminally advancing, to the tissue, a distal end of a driveshaft of a driver, the distal end of the drive shaft being engaged with the anchor; and (ii) subsequently, driving the anchor into the tissue by actuating a release interface at a proximal end of the driver in a manner that releases a torsion spring of the driver to rotate the distal end of the driveshaft.

Example 426. The method according to example 425, further comprising winding up the spring.

Example 427. The method according to any one of examples 425-426, further comprising engaging the driver with the head.

Example 428. The method according to any one of examples 425-427, further comprising, subsequently to advancing the tissue-engaging element into the tissue, disengaging the driver from the anchor and withdrawing the driver from the subject.

Example 429. The method according to any one of examples 425-428, wherein the system or the apparatus further comprises a detent operatively coupled to the driveshaft and to the release interface, such that activating the release interface comprises removing the detent from a position that maintains the tensioned state.

Example 430. The method according to any one of examples 425-429, further comprising, responsively to activating the release interface, unscrewing the tissue-engaging element from the tissue by applying reverse torque to the interface using the driver.

Example 431. A system or an apparatus for anchoring an anchor to tissue, the system or the apparatus comprising a driver that comprises: (i) a driveshaft having a distal end that is configured to be transluminally advanced to the tissue while engaged with the anchor; (ii) a torsion spring; (iii) a winder disposed at a proximal portion of the driver, and operatively coupled to the torsion spring such that operation of the winder winds up the spring; and (iv) a release interface at a proximal end of the driver, operatively coupled to the winder such that actuation of the release interface releases the spring to unwind in a manner that rotates the distal end of the driveshaft.

Example 432. The system or the apparatus according to example 431, wherein the driver further comprises a drive-head at a distal end of the driveshaft, the drive-head configured to engage the anchor.

Example 433. The system or the apparatus according to example 432, further comprising the anchor, wherein the drive-head is configured to fit into a cavity in a head of the anchor.

Example 434. The system or the apparatus according to example 432, further comprising the anchor, wherein the drive-head is configured to define a cavity that fits over a head of the anchor.

Example 435. The system or the apparatus according to any one of examples 431-434, wherein the driver further comprises a detent, configured to maintain the spring in a tensioned state.

Example 436. The system or the apparatus according to example 435, wherein the detent is at the proximal portion of the driver.

Example 437. The system or the apparatus according to example 435, wherein the detent is at a distal portion of the driver.

The present invention is not limited to the examples that have been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description. Further, the treatment techniques, methods, procedures, steps, etc. described or suggested herein or references incorporated herein can be performed on a living animal or on a non-living simulation, such as on a cadaver, cadaver heart, simulator (e.g., with the body parts, tissue, etc. being simulated), etc.

1-20. (canceled)

21. A system for anchoring in a tissue, the system comprising:
an anchor comprising:
a helical tissue-engaging element;
a second tissue-engaging element; and
a head assembly, comprising:
a first head, fixed to a proximal end of the helical tissue-engaging element, and
a second head, fixed to a proximal end of the second tissue-engaging element; and
a driver reversibly interfaces with the head assembly in a manner that drives both the helical tissue-engaging element and the second tissue-engaging element into engagement with the tissue by rotating the first head with respect to the second head.

22. The system according to claim 21, wherein the driver anchors the anchor in the tissue in a manner that the helical tissue-engaging element and the second tissue-engaging element enter the tissue asynchronously.

23. The system according to claim 21, wherein the second head comprises an outer cylinder, and the first head comprises an independently moveable inner core that interfaces with the second head.

24. The system according to claim 21, wherein the first head and the second head form a locking mechanism when the helical tissue-engaging element and the second tissue-engaging element are fully inserted into the tissue.

25. The system according to claim 21, wherein the first head comprises an outer cylinder, and the second head comprises an independently moveable inner core that interfaces with the first head.

26. The system according to claim 21, wherein:
the second head comprises a concavity with an opening in a central region of the concavity;
the helical tissue-engaging element traverses the opening in the central region of the concavity; and
the first head fits rotatably within the concavity of the second head.

27. The system according to claim 26, wherein the opening comprises a set of threads matching a pitch and a handedness of the helical tissue-engaging element.

28. The system according to claim 27, wherein turns of the helical tissue-engaging element advance into the tissue by running along the set of threads.

29. The system according to claim 21, wherein the driver comprises a first component and a second component, the first component engaging the first head, and the second component engaging the second head.

30. The system according to claim 29, wherein the first component fits within a cavity of the first head, and the second component fits over the second head.

31. The system according to claim 21, wherein:
the helical tissue-engaging element has a first handedness and a first pitch, and
the second tissue-engaging element is a second helical tissue-engaging element having a second handedness and a second pitch.

32. The system according to claim 31, wherein the first handedness is the same as the second handedness, and the first pitch is the same as the second pitch.

33. The system according to claim 31, wherein the first handedness is opposite the second handedness, and the first pitch is the same as the second pitch.

34. The system according to claim 21, wherein the first head comprises a runner fixed to the helical tissue-engaging element.

35. The system according to claim 34, wherein the runner advances along turns of the second tissue-engaging element to screw the helical tissue-engaging element into the tissue.

36. The system according to claim 34, wherein the runner rides on turns of the second tissue-engaging element as the driver advances the helical tissue-engaging element into the tissue.

37. The system according to claim 34, wherein the runner and the second head form a locking mechanism when the helical tissue-engaging element and the second tissue-engaging element are fully inserted into the tissue.

38. The system according to claim 34, wherein the runner comprises a T-bar.

39. The system according to claim 34, wherein the runner comprises a flat disc.

40. A system for anchoring in a tissue, the system comprising:
an anchor comprising:
a helical tissue-engaging element;
a second tissue-engaging element; and
a head assembly, comprising:
a first head, fixed to a proximal end of the helical tissue-engaging element, and
a second head, fixed to a proximal end of the second tissue-engaging element; and
a driver that reversibly interfaces with the head assembly to anchor the anchor in the tissue:
such that the helical tissue-engaging element and the second tissue-engaging element are both in engagement with the tissue, and
by screwing the helical tissue-engaging element into the tissue independently of rotation of the second tissue-engaging element.
